SEQUENCE LISTING (24 sequences)

SEQ ID NO: 1 - DNA, 669 bp, Artificial Sequence
  Mimivirus P4H codon optimized nucleotide sequence for E. coli
atggtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcctaa

SEQ ID NO: 2 - protein, 222 aa, Artificial Sequence
  Mimivirus P4H amino acid sequence in E. coli
MVLSKSCVSHFRNVGSLNSRDVNLKDDFSYANIDDPYNKPFVLNNLINPTKCQEIMQFANGKLFDSQVLSGTDKNIRNSQQMWISKNNPMVKPIFENICRQFNVPFDNAEDLQVVRYLPNQYYNEHHDSCCDSSKQCSEFIERGGQRILTVLIYLNNEFSDGHTYFPNLNQKFKPKTGDALVFYPLANNSNKCHPYSLHAGMPVTSGEKWIANLWFRERKFS

SEQ ID NO: 3 - protein, 316 aa, Artificial Sequence
  Mimi virus Protein sequence in Pichia
MRFPSIFTAVLFAASSALAAPVNTTTEDETAQIPAEAVIGYSDLEGDFDVAVLPFSNSTNNGLLFINTTIASIAAKEEGVSLEKREAEAVLSKSCVSHFRNVGSLNSRDVNLKDDFSYANIDDPYNKPFVLNNLINPTKCQEIMQFANGKLFDSQVLSGTDKNIRNSQQMWISKNNPMVKPIFENICRQFNVPFDNAEDLQVVRYLPNQYYNEHHDSCCDSSKQCSEFIERGGQRILTVLIYLNNEFSDGHTYFPNLNQKFKPKTGDALVFYPLANNSNKCHPYSLHAGMPVTSGEKWIANLWFRERKFSHHHHHH

SEQ ID NO: 4 - DNA, 687 bp, Artificial Sequence
  Codon optimized gene sequence (for Pichia)
atggtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcccaccaccaccaccaccactaa

SEQ ID NO: 5 - DNA, 954 bp, Artificial Sequence
  Codon optimized Mimivirus P4H gene sequence with secretion signal (for Pichia)
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctgtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcccaccaccaccaccaccactaataa

SEQ ID NO: 6 - protein, 248 aa, Artificial Sequence
  PBCV-1 protein sequence
MTNKFISYNKMETREYLLTILFVIACFMVLNLERREGFETSDRPGVCDGKYYEKIDGFLSDIECDVLINAAIKKGLIKSEVGGATENDPIKLDPKSRNSEQTWFMPGEHEVIDKIQKKTREFLNSKKHCIDKYNFEDVQVARYKPGQYYYHHYDGDDCDDACPKDQRLATLMVYLKAPEEGGGGETDFPTLKTKIKPKKGTSIFFWVADPVTRKLYKETLHAGLPVKSGEKIIANQWIRAVKHHHHHH

SEQ ID NO: 7 - protein, 259 aa, Artificial Sequence
  Cr-1 protein sequence
MLLLGLVLALAGHVAAAPSSAMMGTGHTVGFGELKEEWRGEVVHLSWSPRAFLLKNFLSDEECDYIVEKARPKMVKSSVVDNESGKSVDSEIRTSTGTWFAKGEDSVISKIEKRVAQVTMIPLENHEGLQVLHYHDGQKYEPHYDYFHDPVNAGPEHGGQRVVTMLMYLTTVEEGGETVLPNAEQKVTGDGWSECAKRGLAVKPIKGDALMFYSLKPDGSNDPASLHGSCPTLKGDKWSATKWIHVAPIGGRHHHHHHH

SEQ ID NO: 8 - protein, 304 aa, Artificial Sequence
  Arabidopsis thaliana protein sequence
MARRGLLISFFAIFSVLLQSSTSLISSSSVFVNPSKVKQVSSKPRAFVYEGFLTELECDHMVSLAKASLKRSAVADNDSGESKFSEVRTSSGTFISKGKDPIVSGIEDKISTWTFLPKENGEDIQVLRYEHGQKYDAHFDYFHDKVNIVRGGHRMATILMYLSNVTKGGETVFPDAEIPSRRVLSENKEDLSDCAKRGIAVKPRKGDALLFFNLHPDAIPDPLSLHGGCPVIEGEKWSATKWIHVDSFDRIVTPSGNCTDMNESCERWAVLGECTKNPEYMVGTTELPGYCRRSCKACHHHHHH

SEQ ID NO: 9 - DNA, 7223 bp, Artificial Sequence
  MMV-398
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgataggctcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcagaaaagggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttattcagaagcgatagagagactgcgctaagcattaatgagattatttttgagcattcgtcaatcaataccaaacaagacaaacggtatgccgacttttggaagtttctttttgaccaactggccgttagcatttcaacgaaccaaacttagttcatcttggatgagatcacgcttttgtcatattaggttccaagacagcgtttaaactgtcagttttgggccatttggggaacatgaaactatttgaccccacactcagaaagccctcatctggagtgatgttcgggtgtaatgcggagcttgttgcattcggaaataaacaaacatgaacctcgccaggggggccaggatagacaggctaataaagtcatggtgttagtagcctaatagaaggaattggaataaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaagctgtctccgatacactcgactaccatccgggtctctcagagaggggaatggcactataaataccgcctccttgcgctctctgccttcatcaatcaaatcatgttctctccaattttgtccttggaaattattttagctttggctactttgcaatctgtcttcgctcaacaggaagcagtagatggtggttgctcacatttaggtcaatcttacgcagatagagatgtatggaaacctgaaccatgtcaaatttgcgtgtgtgactcaggttcagtgctctgcgacgatatcatatgtgacgaccaggaattggactgtccaaacccagagataccattcggtgaatgttgtgctgtttgtccacagccaccaactgctcctacaagacctccaaacggtcaaggtccacaaggtcctaaaggtgatccgggtccacctggtattcctggtagaaatggtgaccctggacctcccggttccccaggtagcccaggatcacctgggcctcctggaatatgtgaatcctgcccaactggtggtcagaactatagcccacaatacgaggcctacgacgtcaaatctggtgttgctggaggaggtattgcaggctaccctggtcccgcagggcccccaggtccgccgggtccgcccggaacatcaggtcatcccggagcccctggtgcaccaggttatcagggaccgcccggagagcctggacaagctggtcccgctggaccccctggtccaccaggtgctattggaccaagtggtcctgccggaaaagacggtgaatccggtagacctggtagacccggcgaaaggggtttcccaggtcctcccggaatgaagggtccagccggtatgcccggttttcctgggatgaagggtcacagaggatttgatggtagaaacggagagaaaggcgaaaccggtgctcccggactgaagggtgaaaacggtgtccctggtgagaacggcgctcctggacctatgggtccacgtggtgctccaggagaaagaggcagaccaggattgcctggtgcagctggtgctagaggtaacgatggtgcccgtggttccgatggacaacccgggccacccggccctccaggtaccgctggatttcctggaagccctggtgctaagggggaggttggtccggctggtagtcccggaagtagcggtgccccaggtcaaagaggcgaaccaggccctcagggtcacgcaggagcacctggaccgcctggtcctcctggttcgaatggttcgcctggaggaaaaggtgaaatggggcccgcaggaatccccggtgcgcctggtcttattggtgccaggggtcctccaggcccgccaggtacaaatggtgtacccggacagcgaggagcagctggtgaacctggtaaaaacggtgccaaaggagatccaggtcctcgtggagagcgtggtgaagctggctctcccggtatcgccggtccaaaaggtgaggacggtaaggacggttcccctggtgagccaggtgcgaacggactgccaggtgcagccggagagcgaggagtcccaggattcaggggaccagccggtgctaacggcttgcctggtgaaaaagggccccctggtgataggggaggacccggtccagcaggccctcgtggagttgctggtgagcctggacgtgacggtttaccaggagggccaggtttgaggggtattcccgggtcccctggcggtcctggatcggatggaaaaccagggccaccaggttcgcagggtgaaacaggacgtccaggcccacccggctcacctggtccaaggggtcagcctggtgtcatgggtttccccggtccaaagggtaatgacggagcaccgggtaaaaatggtgaacgtggtggcccaggtggtccaggaccccaaggtccagctggaaaaaacggtgagacaggtcctcaaggacctccaggacctaccggtcctagcggagataagggagatacgggaccgccaggacctcaaggattgcaaggtttgcctggtacatctggccctcccggagaaaatggtaagcctggagagccaggaccaaaaggcgaagctggagccccaggtatccccggaggtaagggagactcaggtgctccgggtgagcgtggtcctccgggtgccggtggtccacctggacctagaggtggtgccgggccgccaggtcctgaaggtggtaaaggtgctgctggtccaccgggaccgcctggctctgctggtactcctggcttgcagggaatgccaggagagagaggtggacctggaggtcccggtccgaagggtgataaaggggagccaggatcatccggtgttgacggcgcacctggtaaagacggaccaaggggaccaacgggtccaatcggaccaccaggacccgctggccagccaggagataaaggcgagtccggagcacccggtgttcctggtatagctggacccaggggtggtcccggtgaaagaggtgaacagggcccaccgggtcccgccggtttccctggcgcccctggtcaaaatggagaaccaggtgcaaagggcgagagaggagccccaggagaaaagggtgagggaggaccacccggtgctgccggtccagctgggggttcaggtcctgctggaccaccaggtccacagggcgttaaaggtgagagaggaagtccaggtggtcctggagctgctggattcccaggtggccgtggacctcctggtccccctggatcgaatggtaatcctggtccgccaggtagttcgggtgctcctgggaaggacggtccacctggccccccaggtagtaacggtgcacctggtagtccaggtatatccggacctaaaggagattccggtccaccaggcgaaagaggggccccaggcccacagggtccaccaggagcccccggtcctctgggtattgctggtcttactggtgcacgtggactggccggtccacccggaatgcctggagcaagaggttcacctggaccacaaggtattaaaggagagaacggtaaacctggaccttccggtcaaaacggagagcggggacccccaggcccccaaggtctgccaggactagctggtaccgcaggggaaccaggaagagatggaaatccaggttcagacggactacccggtagagatggtgcaccgggggccaagggcgacaggggtgagaatggatctcctggtgcgccaggggcaccaggccacccaggtcccccaggtcctgtgggccctgctggaaagtcaggtgacaggggagagacaggcccggctggtccatctggcgcacccggaccagctggttccagaggcccacctggtccgcaaggccctagaggtgacaagggagagactggagaacgaggtgctatgggtatcaagggtcatagaggttttccgggtaatcccggcgccccaggttctcctggtccagctggccatcaaggtgcagtcggatcgcccggcccagccggtcccaggggccctgttggtccatccggtcctccaggaaaggatggtgcttctggacacccaggacctatcggacctccgggtcctagaggtaatagaggagaacgtggatccgagggtagtcctggtcaccctggtcaacctggcccaccagggcctccaggtgcacccggtccatgttgtggtgcaggcggtgtggctgcaattgctggtgtgggtgctgaaaaggccggcggtttcgctccatattatggtgatggttacattcctgaagctcctagagacggacaagcatacgttagaaaggacggtgagtgggtgttgctgtccaccttcttataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 10 - DNA, 7139 bp, Artificial Sequence
  MMV-589
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgataggctcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcagaaaagggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttattcagaagcgatagagagactgcgctaagcattaatgagattatttttgagcattcgtcaatcaataccaaacaagacaaacggtatgccgacttttggaagtttctttttgaccaactggccgttagcatttcaacgaaccaaacttagttcatcttggatgagatcacgcttttgtcatattaggttccaagacagcgtttaaactgtcagttttgggccatttggggaacatgaaactatttgaccccacactcagaaagccctcatctggagtgatgttcgggtgtaatgcggagcttgttgcattcggaaataaacaaacatgaacctcgccaggggggccaggatagacaggctaataaagtcatggtgttagtagcctaatagaaggaattggaataaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaagctgtctccgatacactcgactaccatccgggtctctcagagaggggaatggcactataaataccgcctccttgcgctctctgccttcatcaatcaaatcatgttctctccaattttgtccttggaaattattttagctttggctactttgcaatctgtcttcgctcaacaggaagcagtagatggtggttgctcacatttaggtcaatcttacgcagatagagatgtatggaaacctgaaccatgtcaaatttgcgtgtgtgactcaggttcagtgctctgcgacgatatcatatgtgacgaccaggaattggactgtccaaacccagagataccattcggtgaatgttgtgctgtttgtccacagccaccaactgctcctacaagacctccaaacggtcaaggtccacaaggtcctaaaggtgatccgggtccacctggtattcctggtagaaatggtgaccctggacctcccggttccccaggtagcccaggatcacctgggcctcctggaatatgtgaatcctgcccaactggtggtcagaactatagcccacaatacgaggcctacgacgtcaaatctggtgttgctggaggaggtattgcaggctaccctggtcccgcagggcccccaggtccgccgggtccgcccggaacatcaggtcatcccggagcccctggtgcaccaggttatcagggaccgcccggagagcctggacaagctggtcccgctggaccccctggtccaccaggtgctattggaccaagtggtcctgccggaaaagacggtgaatccggtagacctggtagacccggcgaaaggggtttcccaggtcctcccggaatgaagggtccagccggtatgcccggttttcctgggatgaagggtcacagaggatttgatggtagaaacggagagaaaggcgaaaccggtgctcccggactgaagggtgaaaacggtgtccctggtgagaacggcgctcctggacctatgggtccacgtggtgctccaggagaaagaggcagaccaggattgcctggtgcagctggtgctagaggtaacgatggtgcccgtggttccgatggacaacccgggccacccggccctccaggtaccgctggatttcctggaagccctggtgctaagggggaggttggtccggctggtagtcccggaagtagcggtgccccaggtcaaagaggcgaaccaggccctcagggtcacgcaggagcacctggaccgcctggtcctcctggttcgaatggttcgcctggaggaaaaggtgaaatggggcccgcaggaatccccggtgcgcctggtcttattggtgccaggggtcctccaggcccgccaggtacaaatggtgtacccggacagcgaggagcagctggtgaacctggtaaaaacggtgccaaaggagatccaggtcctcgtggagagcgtggtgaagctggctctcccggtatcgccggtccaaaaggtgaggacggtaaggacggttcccctggtgagccaggtgcgaacggactgccaggtgcagccggagagcgaggagtcccaggattcaggggaccagccggtgctaacggcttgcctggtgaaaaagggccccctggtgataggggaggacccggtccagcaggccctcgtggagttgctggtgagcctggacgtgacggtttaccaggagggccaggtttgaggggtattcccgggtcccctggcggtcctggatcggatggaaaaccagggccaccaggttcgcagggtgaaacaggacgtccaggcccacccggctcacctggtccaaggggtcagcctggtgtcatgggtttccccggtccaaagggtaatgacggagcaccgggtaaaaatggtgaacgtggtggcccaggtggtccaggaccccaaggtccagctggaaaaaacggtgagacaggtcctcaaggacctccaggacctaccggtcctagcggagataagggagatacgggaccgccaggacctcaaggattgcaaggtttgcctggtacatctggccctcccggagaaaatggtaagcctggagagccaggaccaaaaggcgaagctggagccccaggtatccccggaggtaagggagactcaggtgctccgggtgagcgtggtcctccgggtgccggtggtccacctggacctagaggtggtgccgggccgccaggtcctgaaggtggtaaaggtgctgctggtccaccgggaccgcctggctctgctggtactcctggcttgcagggaatgccaggagagagaggtggacctggaggtcccggtccgaagggtgataaaggggagccaggatcatccggtgttgacggcgcacctggtaaagacggaccaaggggaccaacgggtccaatcggaccaccaggacccgctggccagccaggagataaaggcgagtccggagcacccggtgttcctggtatagctggacccaggggtggtcccggtgaaagaggtgaacagggcccaccgggtcccgccggtttccctggcgcccctggtcaaaatggagaaccaggtgcaaagggcgagagaggagccccaggagaaaagggtgagggaggaccacccggtgctgccggtccagctgggggttcaggtcctgctggaccaccaggtccacagggcgttaaaggtgagagaggaagtccaggtggtcctggagctgctggattcccaggtggccgtggacctcctggtccccctggatcgaatggtaatcctggtccgccaggtagttcgggtgctcctgggaaggacggtccacctggccccccaggtagtaacggtgcacctggtagtccaggtatatccggacctaaaggagattccggtccaccaggcgaaagaggggccccaggcccacagggtccaccaggagcccccggtcctctgggtattgctggtcttactggtgcacgtggactggccggtccacccggaatgcctggagcaagaggttcacctggaccacaaggtattaaaggagagaacggtaaacctggaccttccggtcaaaacggagagcggggacccccaggcccccaaggtctgccaggactagctggtaccgcaggggaaccaggaagagatggaaatccaggttcagacggactacccggtagagatggtgcaccgggggccaagggcgacaggggtgagaatggatctcctggtgcgccaggggcaccaggccacccaggtcccccaggtcctgtgggccctgctggaaagtcaggtgacaggggagagacaggcccggctggtccatctggcgcacccggaccagctggttccagaggcccacctggtccgcaaggccctagaggtgacaagggagagactggagaacgaggtgctatgggtatcaagggtcatagaggttttccgggtaatcccggcgccccaggttctcctggtccagctggccatcaaggtgcagtcggatcgcccggcccagccggtcccaggggccctgttggtccatccggtcctccaggaaaggatggtgcttctggacacccaggacctatcggacctccgggtcctagaggtaatagaggagaacgtggatccgagggtagtcctggtcaccctggtcaacctggcccaccagggcctccaggtgcacccggtccatgttgtggtgcaggcggtgtggctgcaattgctggtgtgggtgctgaaaaggccggcggtttcgctccatattatggttaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 11 - DNA, 10459 bp, Artificial Sequence
  MMV-619
gttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccggaacggaacgtatcttagcatggttgtgcgacagattcactgtgaaagactgttcattatacccacgtttcactgggagatgtaagccttaggtgttttaccctgattagataatacaataaccaacagaaatacgagaatctaaactaatttcgatgattcatttttctttttaccgcgctgcctcttttggcaattctttcacctatattctaccttctctttccttttgttctaaacttattaccagctacatatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggttgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaactgcagtatactgagtttgttaatgatacaataaactgttatagtacatacaattgaaactctcttatctatactgggggaccttctcgcagaatggtataaatatctactaactgactgtcgtacggcctaggggtctcttcttcgattatttgcaggtcggaacatccttcgtctgatgcggatctcctgagacaaagttcacgggtatctagtattctatcagcataaatggaggacctttctaaactaaactttgaatcgtctccagcagcatcctcgcattcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatcttcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcatttaaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtgtctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtttacatcaagctgtctccgatacactcgactaccatccgggtctctcagagaggggaatggcactataaataccgcctccttgcgctctctgccttcatcaatcaaatcatgtcttttgtccaaaagggtacttggttactttttgctctgttgcacccaactgttattctcgcacaacaggaagcagtagatggtggttgctcacatttaggtcaatcttacgcagatagagatgtatggaaacctgaaccatgtcaaatttgcgtgtgtgactcaggttcagtgctctgcgacgatatcatatgtgacgaccaggaattggactgtccaaacccagagataccattcggtgaatgttgtgctgtttgtccacagccaccaactgctcctacaagacctccaaacggtcaaggtccacaaggtcctaaaggtgatccgggtccacctggtattcctggtagaaatggtgaccctggacctcccggttccccaggtagcccaggatcacctgggcctcctggaatatgtgaatcctgcccaactggtggtcagaactatagcccacaatacgaggcctacgacgtcaaatctggtgttgctggaggaggtattgcaggctaccctggtcccgcagggcccccaggtccgccgggtccgcccggaacatcaggtcatcccggagcccctggtgcaccaggttatcagggaccgcccggagagcctggacaagctggtcccgctggaccccctggtccaccaggtgctattggaccaagtggtcctgccggaaaagacggtgaatccggtagacctggtagacccggcgaaaggggtttcccaggtcctcccggaatgaagggtccagccggtatgcccggttttcctgggatgaagggtcacagaggatttgatggtagaaacggagagaaaggcgaaaccggtgctcccggactgaagggtgaaaacggtgtccctggtgagaacggcgctcctggacctatgggtccacgtggtgctccaggagaaagaggcagaccaggattgcctggtgcagctggtgctagaggtaacgatggtgcccgtggttccgatggacaacccgggccacccggccctccaggtaccgctggatttcctggaagccctggtgctaagggggaggttggtccggctggtagtcccggaagtagcggtgccccaggtcaaagaggcgaaccaggccctcagggtcacgcaggagcacctggaccgcctggtcctcctggttcgaatggttcgcctggaggaaaaggtgaaatggggcccgcaggaatccccggtgcgcctggtcttattggtgccaggggtcctccaggcccgccaggtacaaatggtgtacccggacagcgaggagcagctggtgaacctggtaaaaacggtgccaaaggagatccaggtcctcgtggagagcgtggtgaagctggctctcccggtatcgccggtccaaaaggtgaggacggtaaggacggttcccctggtgagccaggtgcgaacggactgccaggtgcagccggagagcgaggagtcccaggattcaggggaccagccggtgctaacggcttgcctggtgaaaaagggccccctggtgataggggaggacccggtccagcaggccctcgtggagttgctggtgagcctggacgtgacggtttaccaggagggccaggtttgaggggtattcccgggtcccctggcggtcctggatcggatggaaaaccagggccaccaggttcgcagggtgaaacaggacgtccaggcccacccggctcacctggtccaaggggtcagcctggtgtcatgggtttccccggtccaaagggtaatgacggagcaccgggtaaaaatggtgaacgtggtggcccaggtggtccaggaccccaaggtccagctggaaaaaacggtgagacaggtcctcaaggacctccaggacctaccggtcctagcggagataagggagatacgggaccgccaggacctcaaggattgcaaggtttgcctggtacatctggccctcccggagaaaatggtaagcctggagagccaggaccaaaaggcgaagctggagccccaggtatccccggaggtaagggagactcaggtgctccgggtgagcgtggtcctccgggtgccggtggtccacctggacctagaggtggtgccgggccgccaggtcctgaaggtggtaaaggtgctgctggtccaccgggaccgcctggctctgctggtactcctggcttgcagggaatgccaggagagagaggtggacctggaggtcccggtccgaagggtgataaaggggagccaggatcatccggtgttgacggcgcacctggtaaagacggaccaaggggaccaacgggtccaatcggaccaccaggacccgctggccagccaggagataaaggcgagtccggagcacccggtgttcctggtatagctggacccaggggtggtcccggtgaaagaggtgaacagggcccaccgggtcccgccggtttccctggcgcccctggtcaaaatggagaaccaggtgcaaagggcgagagaggagccccaggagaaaagggtgagggaggaccacccggtgctgccggtccagctgggggttcaggtcctgctggaccaccaggtccacagggcgttaaaggtgagagaggaagtccaggtggtcctggagctgctggattcccaggtggccgtggacctcctggtccccctggatcgaatggtaatcctggtccgccaggtagttcgggtgctcctgggaaggacggtccacctggccccccaggtagtaacggtgcacctggtagtccaggtatatccggacctaaaggagattccggtccaccaggcgaaagaggggccccaggcccacagggtccaccaggagcccccggtcctctgggtattgctggtcttactggtgcacgtggactggccggtccacccggaatgcctggagcaagaggttcacctggaccacaaggtattaaaggagagaacggtaaacctggaccttccggtcaaaacggagagcggggacccccaggcccccaaggtctgccaggactagctggtaccgcaggggaaccaggaagagatggaaatccaggttcagacggactacccggtagagatggtgcaccgggggccaagggcgacaggggtgagaatggatctcctggtgcgccaggggcaccaggccacccaggtcccccaggtcctgtgggccctgctggaaagtcaggtgacaggggagagacaggcccggctggtccatctggcgcacccggaccagctggttccagaggcccacctggtccgcaaggccctagaggtgacaagggagagactggagaacgaggtgctatgggtatcaagggtcatagaggttttccgggtaatcccggcgccccaggttctcctggtccagctggccatcaaggtgcagtcggatcgcccggcccagccggtcccaggggccctgttggtccatccggtcctccaggaaaggatggtgcttctggacacccaggacctatcggacctccgggtcctagaggtaatagaggagaacgtggatccgagggtagtcctggtcaccctggtcaacctggcccaccagggcctccaggtgcacccggtccatgttgtggtgcaggcggtgtggctgcaattgctggtgtgggtgctgaaaaggccggcggtttcgctccatattatggttaaggcggccgcaaacg

SEQ ID NO: 12 - DNA, 4029 bp, Artificial Sequence
  MMV-644
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgataggctcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcagaaaagggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtatttaaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtgtctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtttacatcaagctgtctccgatacactcgactaccatccgggtctctcagagaggggaatggcactataaataccgcctccttgcgctctctgccttcatcaatcaaatcatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctgtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcccaccaccaccaccaccactaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 13 - DNA, 6068 bp, Artificial Sequence
  MMV-580
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttcctctagaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaaaggaagaatctgcaggaaaagggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttttgttcgattattctccagataaaatcaacaatagttgtttgtaagtaaacgaatcaagatactgaaaatagtttcaaaagcagatcatctgggatttatatatcaggcatcctgctttagttcttttttgaacccaaaggctatctgatgaaaagttgatataggtatgaagaccagaatttgcctagaggctaaccgagacctgaggctaaaaaaggcaggaggaaaagtcctgccaaagataggtatttgaacttgttcgaaaaaggcggaagtttaaacacatggttggagcaagcggcggaatagcggagggatgatacgcagcaaggctgggatcattcgagtttcaaggaacgttagctcaacattcattgactggtaagcgacaactggtttcatctgggtggagttagtctggtgttgggatgctagttgttccccacaattgaaggccagatgaggaggatggtgtggtgataagagatgcaaacagatggttatggccttttgagaacaaagtagacctgtcactcaattgttgtttatatcattgctatttaaatcaggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaatgttctctccaattttgtccttggaaattattttagctttggctactttgcaatctgtcttcgctgtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcccaccaccaccaccaccactaatgaagatctggaggaggctgaggaacctgatcttgaggaggatgacgaccagaaggcagtcaaagatgaactgtgataaggggggccgcgagtcgtgagtaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 14 - DNA, 5869 bp, Artificial Sequence
  MMV-630
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttcctctagaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaaaggaagaatctgcaggaaaagggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttttgttcgattattctccagataaaatcaacaatagttgtttgtaagtaaacgaatcaagatactgaaaatagtttcaaaagcagatcatctgggatttatatatcaggcatcctgctttagttcttttttgaacccaaaggctatctgatgaaaagttgatataggtatgaagaccagaatttgcctagaggctaaccgagacctgaggctaaaaaaggcaggaggaaaagtcctgccaaagataggtatttgaacttgttcgaaaaaggcggaagtttaaacacatggttggagcaagcggcggaatagcggagggatgatacgcagcaaggctgggatcattcgagtttcaaggaacgttagctcaacattcattgactggtaagcgacaactggtttcatctgggtggagttagtctggtgttgggatgctagttgttccccacaattgaaggccagatgaggaggatggtgtggtgataagagatgcaaacagatggttatggccttttgagaacaaagtagacctgtcactcaattgttgtttatatcattgctatttaaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtgtctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtttacatcaagctgtctccgatacactcgactaccatccgggtctctcagagaggggaatggcactataaataccgcctccttgcgctctctgccttcatcaatcaaatcatgttctctccaattttgtccttggaaattattttagctttggctactttgcaatctgtcttcgctgtgctgtcaaagtcctgtgtcagtcactttagaaatgttggatccttgaatagtagggatgtcaatctgaaagatgacttttcctatgctaatattgatgatccctataacaagcctttcgtcctaaataacctaataaaccctaccaagtgtcaagagatcatgcaatttgccaatggcaagttgtttgactcccaagtcctgagtggcacggacaagaacatacgtaactctcaacaaatgtggatatccaagaacaaccctatggtaaaacccattttcgagaacatatgcaggcagtttaacgtaccctttgataatgccgaggacctacaggtcgtccgttacttgcctaatcaatattataatgagcatcatgactcatgctgtgactcctccaagcaatgcagtgaatttatagagaggggcggtcagaggattctgaccgttttaatttacctaaacaacgagttctcagatggacacacgtactttcctaatttaaaccaaaagttcaagcccaagactggtgatgctttggttttttaccctttagccaacaactctaataaatgtcacccatacagtctacacgcaggtatgcccgtcacgtcaggagagaagtggattgctaatctgtggtttcgtgagcgtaagttctcccaccaccaccaccaccactaatgaagatctggaggaggctgaggaacctgatcttgaggaggatgacgaccagaaggcagtcaaagatgaactgtgataaggggggccgcgagtcgtgagtaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 15 - DNA, 60 bp, Artificial Sequence
  primer
gagctcggtaccatgcaccaccaccaccaccacgtgctgtcaaagtcctgtgtcagtcac

SEQ ID NO: 16 - DNA, 41 bp, Artificial Sequence
  Primer
aagcttgaattcttaggagaacttacgctcacgaaaccaca

SEQ ID NO: 17 - DNA, 40 bp, Artificial Sequence
  Primer
gagctcggtaccatggtgctgtcaaagtcctgtgtcagtc

SEQ ID NO: 18 - DNA, 58 bp, Artificial Sequence
  Primer
aagcttgaattcttagtggtggtggtggtggtgggagaacttacgctcacgaaaccac

SEQ ID NO: 19 - DNA, 50 bp, Artificial Sequence
  MM-0579
ctctgccttcatcaatcaaatcatgagattcccatctattttcaccgctg

SEQ ID NO: 20 - DNA, 25 bp, Artificial Sequence
  MM-0580
agcttcggcctctcttttctcgaga

SEQ ID NO: 21 - DNA, 55 bp, Artificial Sequence
  MM-1569
tctcgagaaaagagaggccgaagctgtgctgtcaaagtcctgtgtcagtcacttt

SEQ ID NO: 22 - DNA, 60 bp, Artificial Sequence
  MM-1570
gcaaatggcattctgacatcctcttgattagtggtggtggtggtggtgggagaacttacg

SEQ ID NO: 23 - DNA, 84 bp, Artificial Sequence
  MM-0784
aggaggccatgcacattgtcagaattagaaggttctggctctggttctggctctatgagattcccatctattttcaccgctgtc

SEQ ID NO: 24 - protein, 1220 aa, Artificial Sequence
  Protein sequence in PP681
MFSPILSLEIILALATLQSVFAQQEAVDGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGPPGSPGSPGSPGPPGICESCPTGGQNYSPQYEAYDVKSGVAGGGIAGYPGPAGPPGPPGPPGTSGHPGAPGAPGYQGPPGEPGQAGPAGPPGPPGAIGPSGPAGKDGESGRPGRPGERGFPGPPGMKGPAGMPGFPGMKGHRGFDGRNGEKGETGAPGLKGENGVPGENGAPGPMGPRGAPGERGRPGLPGAAGARGNDGARGSDGQPGPPGPPGTAGFPGSPGAKGEVGPAGSPGSSGAPGQRGEPGPQGHAGAPGPPGPPGSNGSPGGKGEMGPAGIPGAPGLIGARGPPGPPGTNGVPGQRGAAGEPGKNGAKGDPGPRGERGEAGSPGIAGPKGEDGKDGSPGEPGANGLPGAAGERGVPGFRGPAGANGLPGEKGPPGDRGGPGPAGPRGVAGEPGRDGLPGGPGLRGIPGSPGGPGSDGKPGPPGSQGETGRPGPPGSPGPRGQPGVMGFPGPKGNDGAPGKNGERGGPGGPGPQGPAGKNGETGPQGPPGPTGPSGDKGDTGPPGPQGLQGLPGTSGPPGENGKPGEPGPKGEAGAPGIPGGKGDSGAPGERGPPGAGGPPGPRGGAGPPGPEGGKGAAGPPGPPGSAGTPGLQGMPGERGGPGGPGPKGDKGEPGSSGVDGAPGKDGPRGPTGPIGPPGPAGQPGDKGESGAPGVPGIAGPRGGPGERGEQGPPGPAGFPGAPGQNGEPGAKGERGAPGEKGEGGPPGAAGPAGGSGPAGPPGPQGVKGERGSPGGPGAAGFPGGRGPPGPPGSNGNPGPPGSSGAPGKDGPPGPPGSNGAPGSPGISGPKGDSGPPGERGAPGPQGPPGAPGPLGIAGLTGARGLAGPPGMPGARGSPGPQGIKGENGKPGPSGQNGERGPPGPQGLPGLAGTAGEPGRDGNPGSDGLPGRDGAPGAKGDRGENGSPGAPGAPGHPGPPGPVGPAGKSGDRGETGPAGPSGAPGPAGSRGPPGPQGPRGDKGETGERGAMGIKGHRGFPGNPGAPGSPGPAGHQGAVGSPGPAGPRGPVGPSGPPGKDGASGHPGPIGPPGPRGNRGERGSEGSPGHPGQPGPPGPPGAPGPCCGAGGVAAIAGVGAEKAGGFAPYYG

(19) United States
(12) Patent Application Publication
(10) Pub. No.: US 2023/0174955 A1
(43) Pub. Date: Jun. 8, 2023

(54) MONOMERIC PROTEINS FOR HYDROXYLATING AMINO ACIDS AND PRODUCTS

(71) Applicant: Modern Meadow, Inc., Nutley, NJ (US)

(72) Inventors: Lixin DAI, Nutley, NJ (US); Poonam SRIVASTAVA, Nutley, NJ (US); Sumreet Singh JOHAR, Nutley, NJ (US); Shashwat Mohan VAJPEYI, Nutley, NJ (US); Mahadevan LAKSHMINARASIMHAN, Nutley, NJ (US); James Egan WELCH, Nutley, NJ (US)

(73) Assignee: Modern Meadow, Inc., Nutley, NJ (US)

(21) Appl. No.: 17/759,881

(22) PCT Filed: Feb. 12, 2021
(86) PCT No.: PCT/US2021/017861

§ 371 Date: Aug. 1, 2022

Related U.S. Application Data
(60) Provisional application No. 62/976,632, filed on Feb. 14, 2020.

(51) Int. Cl. C12N 9/02; C07K 14/78

(52) U.S. Cl. CPC C12N 9/0071; C07K 14/78; C12Y 114/11002

(57) ABSTRACT

The disclosure herein provides a monomeric prolyl 4-hydroxylase. A microorganism including a monomeric prolyl 4-hydroxylase is provided. The disclosure provides a microorganism including a monomeric prolyl 4-hydroxylase; and another protein to be hydroxylated. A method for providing skincare benefits including applying the fusion protein of the present disclosure on skin is also taught.

### REFERENCE TO SEQUENCE LISTING SUBMITTED ELECTRONICALLY

The content of the electronically submitted sequence (Name 4431-064PC01_SL_ST25.txt; Size: 82,152 bytes; and Date of Creation: Feb. 10, 2021) is incorporated herein by reference in its entirety.

### FIELD

Described herein are monomeric prolyl 4-hydroxylase proteins and their use in fermentation, methods for production of said proteins, and methods for in vitro and in vivo hydroxylation of proteins.

### BACKGROUND

There is an entire industry using microorganisms to make compounds for commercial applications. The microorganisms are typically engineered with DNA necessary to make the compounds. Examples of these microorganisms include yeast and bacteria. Compounds that are made include drugs, fragrances, flavors, proteins and the like.

Engineered proteins are created through protein engineering, mutagenesis and protein evolution. One purpose of creating engineered proteins in drug development is to improve their activity under various reaction conditions.

### SUMMARY

In some embodiments, this disclosure provides a yeast host cell comprising a recombinant monomeric prolyl 4-hydroxylase. In some embodiments, the monomeric prolyl 4-hydroxylase can be secreted. In certain embodiments, the recombinant monomeric prolyl 4-hydroxylase can be from a virus, algae, or a plant. In some embodiments, the recombinant monomeric prolyl 4-hydroxylase can be from mimivirus. In one embodiment, the recombinant monomeric prolyl 4-hydroxylase can be from Arabidopsis thaliana. In some embodiments, the recombinant monomeric prolyl 4-hydroxylase can be from C. reinhardtii. In some embodiments, the recombinant monomeric prolyl 4-hydroxylase can be from Paramecium bursaria Chlorella virus-1. In some embodiment, the recombinant monomeric prolyl 4-hydroxylase can have at least 80% identical to a prolyl 4-hydroxylase selected from the group consisting of: SEQ ID NOs: 2, 3, 6, 7 and 8. In certain embodiment, the yeast can be Pichia.

In some embodiments, the yeast host cell can further comprise a second protein to be hydroxylated. In certain embodiments, the second protein can be selected from the group consisting of: collagen, recombinant collagen, and collagen-like proteins.

In some embodiments, this disclosure provides a microorganism comprising a recombinant monomeric prolyl 4-hydroxylase, wherein the recombinant monomeric prolyl 4-hydroxylase can be from algae or a plant. In certain embodiments, the monomeric prolyl 4-hydroxylase can be secreted. In some embodiments, the recombinant monomeric prolyl 4-hydroxylase can be from Arabidopsis thaliana. In certain embodiments, the recombinant monomeric prolyl 4-hydroxylase can be from C. reinhardtii. In some embodiments, the recombinant monomeric prolyl 4-hydroxylase can be at least 80% identical to a prolyl 4-hydroxylase selected from the group consisting of: SEQ ID NOs: 7 and 8.

In some embodiments, the microorganism can be a yeast or a bacteria. In some embodiments, the microorganism can be E. coli. In other embodiments, the microorganism can be Pichia.

In some embodiments, the microorganism can further comprise a second protein to be hydroxylated. In some embodiments, the second protein can be selected from the group consisting of: collagen, recombinant collagen, and collagen-like proteins.

In some embodiments, this disclosure provides a method of producing a recombinant monomeric prolyl 4-hydroxylase, comprising purifying the recombinant monomeric prolyl 4-hydroxylase from a yeast host cell disclosed herein.

In some embodiments, this disclosure provides a method of producing a recombinant monomeric prolyl 4-hydroxylase, comprising purifying the recombinant monomeric prolyl 4-hydroxylase from a microorganism disclosed herein.

In some embodiments, this disclosure provides an in vitro method for hydroxylating a protein comprising: lysing a microorganism comprising a protein to be hydroxylated to create a lysate; adding a specific concentration of a monomeric prolyl 4-hydroxylase to the lysate; and incubating the lysate and the monomeric prolyl 4-hydroxylase in reaction conditions that promote the hydroxylation of the protein by the a monomeric prolyl 4-hydroxylase.

In certain embodiments, this disclosure provides an in vitro method for hydroxylating a protein comprising: lysing a first microorganism comprising a protein to be hydroxylated to create a lysate; adding a specific concentration of a monomeric prolyl 4-hydroxylase to the lysate; and incubating the lysate and the monomeric prolyl 4-hydroxylase in reaction conditions that promote the hydroxylation of the protein by the a monomeric prolyl 4-hydroxylase.

In some embodiments, this disclosure provides an in vitro method for hydroxylating a protein comprising: adding a specific concentration of a monomeric prolyl 4-hydroxylase purified from a yeast host cell disclosed herein to a reaction mixture; adding a specific concentration of a protein to be hydroxylated to the reaction mixture; and incubating the reaction micture under reaction conditions that promote hydroxylation of the protein by the a monomeric prolyl 4-hydroxylase.

In some embodiments, this disclosure provides an in vitro method for hydroxylating a protein comprising: adding a specific concentration of a monomeric prolyl 4-hydroxylase purified from a microorganism disclosed herein to a reaction mixture; adding a specific concentration of a protein to be hydroxylated to the reaction mixture; and incubating the reaction micture under reaction conditions that promote hydroxylation of the protein by the a monomeric prolyl 4-hydroxylase.

In certain embodiments, this disclosure provides an ex vivo method for hydroxylating a protein comprising: lysing a microorganism disclosed herein to create a lysate; incubating the lysate and a protein to be hydroxylated under reaction conditions that promote hydroxylation of the protein by the monomeric prolyl 4-hydroxylase.

In some embodiments, this disclosure provides an ex vivo method for hydroxylating a protein comprising: lysing a yeast host cell to create a lysate; incubating the lysate and a a protein to be hydroxylated under reaction conditions that promote hydroxylation of a protein in the lysate by the monomeric prolyl 4-hydroxylase.

In certain embodiments, this disclosure provides an ex vivo method for hydroxylating a protein comprising: lysing a microorganism comprising a monomeric prolyl 4-hydroxylase to create a first lysate; lysing a second microorganism comprising a protein to be hydroxylated to create a second lysate; and incubating the first lysate and the second lysate under reaction conditions that promote hydroxylation of the protein by the monomeric prolyl 4-hydroxylase.

In some embodiments, this disclosure provides an ex vivo method for hydroxylating a protein comprising: lysing a yeast host cell comprising a recombinant monomeric prolyl-4 hydroxylase to create a yeast host cell lysate; lysing a microorganism comprising a protein to be hydroxylated to create a protein containing lysate; and incubating yeast host cell lysate and the protein containing lysate under reaction conditions that promote hydroxylation of the protein by the monomeric prolyl 4-hydroxylase.

### FIGURES

FIG. 1 depicts a plasmid map of MMV-570

FIG. 2 depicts a method of purifying mimi-virus P4H from E.coli.

FIG. 3 depicts a plasmid map of MMV-644.

FIG. 4 depicts a plasmid map of MMV-398.

FIG. 5 depicts a plasmid map of MMV-580.

FIG. 6 depicts the in vivo hydroxylation of collagen by mimi-virus P4H in Pichia.

FIG. 7 depicts the procedure of ex vivo hydroxylation of collagen by mimi-virus P4H.

FIG. 8 depicts the ex vivo hydroxylation of collagen with secreted mimi-virus P4H in Pichia

FIG. 9 depicts a plasmid map of MMV-589.

FIG. 10 depicts a plasmid map of MMV630.

FIG. 11 depicts the co-expression of collagen with mimi-virus P4H in Pichia.

FIG. 12 depicts the ex vivo hydroxylation with collagen/mimi-virus P4H co-expression Pichia strain.

FIG. 13 depicts a qSDS gene after a high-low pH purification.

FIG. 14 depicts a plasmid map of MMV-619.

FIG. 15 depicts a plasmid map of MMV-620.

FIG. 16 depicts the expression of mimi-virus P4H as secreted protein in Pichia.

FIG. 17 depicts the expression of mimi-virus P4H as secreted protein in Pichia -time course.

FIG. 18 depicts the procedure of ex vivo hydroxylation of collagen with secreted mimi-virus P4H in Pichia.

FIG. 19 depicts the ex vivo hydroxylation of collagen with secreted mimi-virus P4H in Pichia.

FIG. 20 depicts the procedure of ex vivo hydroxylation of collagen with secreted mimi-virus P4H in Pichia .

FIG. 21 depicts the ex vivo hydroxylation of collagen with secreted mimi-virus P4H in Pichia.

### DETAILED DESCRIPTION

### Definitions

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used in the claims, “comprising” or “comprises” is an open-ended transitional phrase. A list of elements following the transitional phrase “comprising” is a non-exclusive list, such that elements in addition to those specifically recited in the list can also be present. As used herein, the terms “includes,” “including,” “has,” “having” or any other variation thereof, are intended to cover a non-exclusive inclusion

Further, unless expressly stated to the contrary, “or” and “and/or” refers to an inclusive and not to an exclusive. For example, a condition A or B, or A and/or B, is satisfied by any one of the following: A is true (or present) and B is false (or not present), A is false (or not present) and B is true (or present), and both A and B are true (or present).

When the term “about” is used, it is used to mean a certain effect or result can be obtained within a certain tolerance, and the skilled person knows how to obtain the tolerance. When the term “about” is used in describing a value or an end-point of a range, the disclosure should be understood to include the specific value or end-point referred to. In certain embodiments, “about” can mean a range of up to 10% (i.e., ±10%).

Any numerical range recited herein is intended to include all sub-ranges subsumed therein. Where a range of numerical values is recited herein, comprising upper and lower values, unless otherwise stated in specific circumstances, the range is intended to include the endpoints thereof, and all integers and fractions within the range. It is not intended that the scope of the claims be limited to the specific values recited when defining a range. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. Finally, when the term “about” is used in describing a value or an end-point of a range, the disclosure should be understood to include the specific value or end-point referred to. Whether or not a numerical value or end-point of a range recites “about,” the numerical value or end-point of a range is intended to include two embodiments: one modified by “about,” and one not modified by “about.”

As used herein “collagen” refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX. The term collagen as used herein also refers to collagen prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties) Unless otherwise specified, collagen of any type, whether naturally occurring or prepared using recombinant techniques, can be used in any of the embodiments described herein. That said, in some embodiments, the composite materials described herein can be prepared using Bovine Type I collagen.

Collagens are characterized by a repeating triplet of amino acids, -(Gly-X-Y)n-, so that approximately one-third of the amino acid residues in collagen are glycine. X is often proline and Y is often hydroxyproline. Thus, the structure of collagen may consist of three intertwined peptide chains of differing lengths. Different animals may produce different amino acid compositions of the collagen, which may result in different properties (and differences in the resulting leather). Collagen triple helices (also called monomers or tropocollagen) can be produced from alpha-chains of about 1050 amino acids long, so that the triple helix takes the form of a rod of about approximately 300 nm long, with a diameter of approximately 1.5 nm. In the production of extracellular matrix by fibroblast skin cells, triple helix monomers can be synthesized and the monomers may self-assemble into a fibrous form. These triple helices can be held together by electrostatic interactions (including salt bridging), hydrogen bonding, Van der Waals interactions, dipole-dipole forces, polarization forces, hydrophobic interactions, and covalent bonding. Triple helices can be bound together in bundles called fibrils, and fibrils can further assemble to create fibers and fiber bundles. In some embodiments, fibrils can have a characteristic banded appearance due to the staggered overlap of collagen monomers. This banding can be called “D-banding.” The bands are created by the clustering of basic and acidic amino acids, and the pattern is repeated four times in the triple helix (D-period). (See, e.g., Covington, A., Tanning Chemistry: The Science of Leather (2009)) The distance between bands can be approximately 67 nm for Type 1 collagen. These bands can be detected using diffraction Transmission Electron Microscope (TEM), which can be used to access the degree of fibrillation in collagen. Fibrils and fibers typically branch and interact with each other throughout a layer of skin. Variations of the organization or crosslinking of fibrils and fibers can provide strength to a material disclosed herein. In some embodiments, protein is formed, but the entire collagen structure is not triple helical. In certain embodiments, the collagen structure can be about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or 100% triple helical.

Regardless of the type of collagen, all are formed and stabilized through a combination of physical and chemical interactions including electrostatic interactions (including salt bridging), hydrogen bonding, Van der Waals interactions, dipole-dipole forces, polarization forces, hydrophobic interactions, and covalent bonding often catalyzed by enzymatic reactions. For Type I collagen fibrils, fibers, and fiber bundles, its complex assembly is achieved in vivo during development and is critical in providing mechanical support to the tissue while allowing for cellular motility and nutrient transport.

Various distinct collagen types have been identified in vertebrates, including bovine, ovine, porcine, chicken, and human collagens. Generally, the collagen types are numbered by Roman numerals, and the chains found in each collagen type are identified by Arabic numerals. Detailed descriptions of structure and biological functions of the various different types of naturally occurring collagens are generally available in the art; see, e.g., Ayad et al. (1998) The Extracellular Matrix Facts Book, Academic Press, San Diego, CA; Burgeson, R E., and Nimmi (1992) “Collagen types: Molecular Structure and Tissue Distribution” in Clin. Orthop. 282:250-272; Kielty, C. M. et al. (1993) “The Collagen Family: Structure, Assembly And Organization In The Extracellular Matrix,” Connective Tissue And Its Heritable Disorders, Molecular Genetics, And Medical Aspects, Royce, P. M. and B. Steinmann eds., Wiley-Liss, NY, pp. 103-147; and Prockop, D.J- and K.I. Kivirikko (1995) “Collagens: Molecular Biology, Diseases, and Potentials for Therapy,” Annu. Rev. Biochem., 64:403-434.) In some embodiments, the sequence can be a sequence that is about 80%, about 85%, about 90%, about 95%, about 96%, about 97%, about 98%, about 99% or 100% identical to the collagen sequence of SEQ ID NO: 24.

Type I collagen is the major fibrillar collagen of bone and skin, comprising approximately 80-90% of an organism’s total collagen. Type I collagen is the major structural macromolecule present in the extracellular matrix of multicellular organisms and comprises approximately 20% of total protein mass. Type I collagen is a heterotrimeric molecule comprising two α1(I) chains and one α2(I) chain, encoded by the COL1A1 and COL1A2 genes, respectively. Other collagen types are less abundant than type I collagen, and exhibit different distribution patterns. For example, type II collagen is the predominant collagen in cartilage and vitreous humor, while type III collagen is found at high levels in blood vessels and to a lesser extent in skin.

Type II collagen is a homotrimeric collagen comprising three identical al(II) chains encoded by the COL2A1 gene. Purified type II collagen can be prepared from tissues by, methods known in the art, for example, by procedures described in Miller and Rhodes (1982) Methods In Enzymology 82:33-64.

Type III collagen is a major fibrillar collagen found in skin and vascular tissues. Type III collagen is a homotrimeric collagen comprising three identical α1(III) chains encoded by the COL3A1 gene. Methods for purifying type III collagen from tissues can be found in, for example, Byers et al. (1974) Biochemistry 13:5243-5248; and Miller and Rhodes, supra.

Type IV collagen is found in basement membranes in the form of sheets rather than fibrils. Most commonly, type IV collagen contains two α1(IV) chains and one α2(IV) chain. The particular chains comprising type IV collagen are tissue-specific. Type IV collagen can be purified using, for example, the procedures described in Furuto and Miller (1987) Methods in Enzymology, 144:41-61, Academic Press.

Type V collagen is a fibrillar collagen found in, primarily, bones, tendon, cornea, skin, and blood vessels. Type V collagen exists in both homotrimeric and heterotrimeric forms. One form of type V collagen is a heterotrimer of two α1(V) chains and one α2(V) chain. Another form of type V collagen is a heterotrimer of α1(V), α2(V), and α3(V) chains. A further form of type V collagen is a homotrimer of α1(V). Methods for isolating type V collagen from natural sources can be found, for example, in Elstow and Weiss (1983) Collagen Rel. Res. 3:181-193, and Abedin et al. (1982) Biosci. Rep. 2:493-502.

Type VI collagen has a small triple helical region and two large non-collagenous remainder portions. Type VI collagen is a heterotrimer comprising α1(VI), α2(VI), and α3(VI) chains. Type VI collagen is found in many connective tissues. Descriptions of how to purify type VI collagen from natural sources can be found, for example, in Wu et al. (1987) Biochem. J. 248:373-381, and Kielty et al. (1991) J. Cell Sci. 99:797-807.

Type VII collagen is a fibrillar collagen found in particular epithelial tissues. Type VII collagen is a homotrimeric molecule of three α1(VII) chains. Descriptions of how to purify type VII collagen from tissue can be found in, for example, Lunstrum et al. (1986) J. Biol. Chem. 261:9042-9048, and Bentz et al. (1983) Proc. Natl. Acad. Sci. USA 80:3168-3172. Type VIII collagen can be found in Descemet’s membrane in the cornea. Type VIII collagen is a heterotrimer comprising two α1(VIII) chains and one α2(VIII) chain, although other chain compositions have been reported. Methods for the purification of type VIII collagen from nature can be found, for example, in Benya and Padilla (1986) J. Biol. Chem. 261:4160-4169, and Kapoor et al. (1986) Biochemistry 25:3930-3937.

Type IX collagen is a fibril-associated collagen found in cartilage and vitreous humor. Type IX collagen is a heterotrimeric molecule comprising α1(IX), α2(IX), and α3 (IX) chains. Type IX collagen has been classified as a FACIT (Fibril Associated Collagens with Interrupted Triple Helices) collagen, possessing several triple helical domains separated by non-triple helical domains. Procedures for purifying type IX collagen can be found, for example, in Duance, et al. (1984) Biochem. J. 221:885-889; Ayad et al. (1989) Biochem. J. 262:753-761; and Grant et al. (1988) The Control of Tissue Damage, Glauert, A. M., ed., Elsevier Science Publishers, Amsterdam, pp. 3-28.

Type X collagen is a homotrimeric compound of α1(X) chains. Type X collagen has been isolated from, for example, hypertrophic cartilage found in growth plates. (See, e.g., Apte et al. (1992) Eur J Biochem 206 (1):217-24.)

Type XI collagen can be found in cartilaginous tissues associated with type II and type IX collagens, and in other locations in the body. Type XI collagen is a heterotrimeric molecule comprising α1(XI), α2(XI), and α3(XI) chains. Methods for purifying type XI collagen can be found, for example, in Grant et al., supra.

Type XII collagen is a FACIT collagen found primarily in association with type I collagen. Type XII collagen is a homotrimeric molecule comprising three α1(XII) chains. Methods for purifying type XII collagen and variants thereof can be found, for example, in Dublet et al. (1989) J. Biol. Chem. 264:13150-13156; Lunstrum et al. (1992) J. Biol. Chem. 267:20087-20092; and Watt et al. (1992) J. Biol. Chem. 267:20093-20099.

Type XIII is a non-fibrillar collagen found, for example, in skin, intestine, bone, cartilage, and striated muscle. A detailed description of type XIII collagen can be found, for example, in Juvonen et al. (1992) J. Biol. Chem. 267: 24700-24707.

Type XIV is a FACIT collagen characterized as a homotrimeric molecule comprising α1(XIV) chains. Methods for isolating type XIV collagen can be found, for example, in Aubert-Foucher et al. (1992) J. Biol. Chem. 267:15759-15764, and Watt et al., supra.

Type XV collagen is homologous in structure to type XVIII collagen. Information about the structure and isolation of natural type XV collagen can be found, for example, in Myers et al. (1992) Proc. Natl. Acad. Sci. USA 89:10144-10148; Huebner et al. (1992) Genomics 14:220-224; Kivirikko et al. (1994) J. Biol. Chem. 269:4773-4779; and Muragaki, J. (1994) Biol. Chem. 264:4042-4046.

Type XVI collagen is a fibril-associated collagen, found, for example, in skin, lung fibroblast, and keratinocytes. Information on the structure of type XVI collagen and the gene encoding type XVI collagen can be found, for example, in Pan et al. (1992) Proc. Natl. Acad. Sci. USA 89:6565-6569; and Yamaguchi et al. (1992) J. Biochem. 112:856-863.

Type XVII collagen is a hemidesmosal transmembrane collagen, also known at the bullous pemphigoid antigen. Information on the structure of type XVII collagen and the gene encoding type XVII collagen can be found, for example, in Li et al. (1993) J. Biol. Chem. 268(12):8825-8834; and McGrath et al. (1995) Nat. Genet. 11(1):83-86.

Type XVIII collagen is similar in structure to type XV collagen and can be isolated from the liver. Descriptions of the structures and isolation of type XVIII collagen from natural sources can be found, for example, in Rehn and Pihlajaniemi (1994) Proc. Natl. Acad. Sci USA 91:4234-4238; Oh et al. (1994) Proc. Natl. Acad. Sci USA 91:4229-4233; Rehn et al. (1994) J. Biol. Chem. 269:13924-13935; and Oh et al. (1994) Genomics 19:494-499.

Type XIX collagen is believed to be another member of the FACIT collagen family, and has been found in mRNA isolated from rhabdomyosarcoma cells. Descriptions of the structures and isolation of type XIX collagen can be found, for example, in Inoguchi et al. (1995) J. Biochem. 117:137-146; Yoshioka et al. (1992) Genomics 13:884-886; and Myers et al., J. Biol. Chem. 289:18549-18557 (1994).

Type XX collagen is a newly found member of the FACIT collagenous family, and has been identified in chick cornea. (See, e.g., Gordon et al. (1999) FASEB Journal 13:A1119; and Gordon et al. (1998), IOVS 39:S1128.)

In the context of the present application a “variant” includes an amino acid sequence having at least 70%, 75%, 80%, 85%, 87.5%, 90%, 92.5%, 95%, 97.5%, 98%, or 99% sequence identity, or similarity to a reference amino acid, such as a monomeric P4H amino acid sequence or an amino acid of selected from any one of SEQ ID NOs: 2, 3, 6, 7 and 8, using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting uses an Expect Threshold of 10, a Word Size of 3, BLOSUM 62 as a matrix, and Gap Penalty of 11 (Existence) and 1 (Extension) and a conditional compositional score matrix adjustment. In typical embodiments, the “variant” retains prolyl-4-hydroxylase activity.

### Hydroxylation of Proline and Lysine Residues in a Protein (e.g., Collagen)

The principal post-translational modifications to protein polypeptides that contain proline and lysine residues, such as collagen, are 1) hydroxylation of proline and lysine residues to yield 4-hydroxyproline, 3-hydroxyproline (Hyp), and hydroxylysine (Hyl); and 2) glycosylation of hydroxylysyl residues. These modifications are catalyzed by three hydroxylases: prolyl 4-hydroxylase, prolyl 3-hydroxylase, and lysyl hydroxylase; and two glycosyl transferases, respectively. In vivo these reactions occur until the polypeptides form the triple-helical collagen structure.

### ProlylHydroxylase

The “prolyl-4-hydroxylase” or “P4H” enzyme catalyzes hydroxylation of proline residues to (2S,4R)-4-hydroxyproline (Hyp). See, Gorres, et al., Critical Reviews in Biochemistry and Molecular Biology 45 (2): (2010), which is incorporated by reference in its entierty. In collagen and related proteins, prolyl 4-hydroxylase catalyzes the formation of 4-hydroxyproline, whichis necessary for the proper three-dimensional folding of newly synthesized procollagen chains.

Monomeric prolyl-4-hydroxylase enzymes are a group of enzymes that function as a single unit (as opposed to animal P4H enzymes that functions as a heterotetramer). The monomeric P4H enzymes are typically much smaller in size (20-50 kD) than the P4H tetramer (120 kD). Monomeric P4H enzymes can be found in, and isolated from, bacteria, algae, plants, and viruses,

In some embodiments, the present disclosure provides a recombinant host cell comprising a recombinant monomeric P4H enzyme. In certain embodiments, the recombinant monomeric P4H enzyme in the host cell is from a virus, an algae, or a plant. In some embodiments, the recombinant monomeric P4H enzyme in the host cell is from mimivirus. In certain embodiments, the recombinant monomeric P4H enzyme in the host cell is from Arabidopsis thaliana. In another embodiment, the recombinant monomeric P4H enzyme in the host cell is from C. reinhardtii. In some embodiments, the recombinant monomeric P4H enzyme in the host cell is from Paramecium bursaria Chlorella virus-1. Isoforms, orthologs, variants, fragments and prolyl-4-hydroxylases from other sources can also be used in the host cell as long as they retain hydroxylase activity in a host cell. In certain embodiments, the recombinant monomeric P4H enzyme in the host cell can have an amino acid sequence selected from the group consisting of SEQ ID NOs: 2, 3, 6, 7 and 8. In some embodiments, the recombinant monomeric P4H enzyme in the host cell can have a sequence that is about 80%, about 85%, about 90%, about 95%, or about 99% identical to a sequence selected from SEQ ID NOs: 2, 3, 6, 7 and 8. In some embodiments, the recombinant monomeric P4H enzyme in the host cell has an amino acid sequence that is a variant of any sequence disclosed herein.

In some embodiments, host cells are engineered to overproduce prolyl-4-hydroxylase. For example, a polynucleotide encoding the prolyl-4-hydroxylase, an isoform thereof, an ortholog thereof, a variant thereof, or a fragment thereof that expresses prolyl-4-hydroxylase activity, can be incorporated into an expression vector. In some embodiments, the expression vector containing the polynucleotide encoding the prolyl-4-hydroxylase, the isoform thereof, the ortholog thereof, the variant thereof, or the fragment thereof, can be under the control of an inducible promoter. Suitable host cells, expression vectors, and promoters are described below.

DNA encoding the monomeric P4H enzyme can be transformed or transfected into an organism. Suitable organisms include, but are not limited to, yeast, bacteria, fungi and the like. In some embodiments, the bacteria can be Bacillus or Escherichia coli. In some embodiments, the microorganism can be a filamentous fungi. In some embodiments, the organism can be yeast. In certain embodiments, the yeast can be Pichia pastoris. In some embodiments, the monomeric P4H enzyme can be used in a method for in vitro hydroxylation of proteins. In some embodiments, monomeric P4H enzyme can be used in a method for in vivo hydroxylation of proteins. In some embodiments, the monomeric P4H enzyme can be used in a method for ex vivo hydroxylation of proteins.

In certain embodiments, monomeric P4H enzyme expressed by a host cell can be secreted.

In some embodiments, monomeric P4H enzyme can be used to hydroxylate proteins in vitro. Microorganisms that contain protein such as collagen can be lysed creating a lysate. The lysate can be processed to create purified proteins. Monomeric P4H enzyme can be added to purified samples of protein or added to the lysate. In some embodiments, co-factors for the hydroxylation reaction can include one or more of ascorbic acid/sodium ascorbate, or an iron (II) containing species, for example FeSO₄. In other embodiments, co-factors for hydroxylation reaction can include alpha-Ketoglutarate (AKG or 2-oxoglutarate) and/or molecular oxygen. In some embodiments, the substrate for the hydroxylation reaction can be collagen. In some embodiments, bovine serum albumin and/or catalase can be added to the reaction to promote hydroxylation efficiency. In some embodiments, the catalase can be bovine catalase (Available from SigmaAldrich: Catalog Number C40).

In some embodiments, the hydroxylation reaction can be performed at a temperature ranging from about 16° C. to about 40° C., for example about 32° C. In some embodiments, the hydroxylation reaction can be performed at about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or at about 40° C.

The amount of monomeric P4H enzyme added to the hydroxylation reaction can range from about 0.05 uM to about 20 uM, for example about 5 uM. In some embodiments, the amount of monomeric P4H enzyme added can be about 0.05 uM, about 0.1 uM, about 0.15 uM, about 0.2 uM, about 0.25 uM, about 0.3 uM, about 0.35 uM, about 0.4 uM, about 0.5 uM, about 0.6 uM, about 0.7 uM, about 0.8 uM, about 0.9 uM, about 1.0 uM, about 1.1 uM, about 1.2 uM, about 1.3 uM, about 1.4 uM, about 1.5 uM, about 1.6 uM, about 1.7 uM, about 1.8 uM, about 1.9 uM, about 2.0 uM, about 2.5 uM, about 3.0 uM, about 3.5 uM, about 4.0 uM, about 4.5 uM, about 5 uM, about 7 uM, about 10 uM, about 15 uM, or about 20 uM.

In some embodiments, the hydroxylation reaction can take place at a pH ranging from about 5 to about 12, for example about 7.5. In some embodiments, the pH can be about 5.0, about 5.5, about 6, about 6.5, about 7, about 7.5, about 8, about 8.5, about 9.0, about 9.5, about 10.0, about 10.5, about 11, about 11.5, or about 12.

In some embodiments, the hydroxylation reaction can take place over about 30 mins to about 5 hours, for example about 1 hour. In some embodiments, the hydroxylation can take place over about 30 minutes, about 45 minutes, about 1 hour, about 1.5 hours, about 2 hours, about 2.5 hours, about 3 hours, about 3.5 hours, about 4 hours, about 4.5 hours, or about 5 hours. In certain embodiments, and after the reaction is complete or has proceeded for a sufficient amount of time, the monomeric P4H enzyme can be inactivated by adding an acid to lower the pH of the solution to about 4. Alternatively, 50% - 80% methanol (by volume) can be added to inactive the enzyme. In some embodiments, the in vitro hydroxylation can be performed using any method disclosed in U.S. Pat. No. 7,932,053, which is incorporated herein by reference in its entirety.

In some embodiments, the monomeric P4H enzyme can be used to hydroxylate proteins ex vivo. Microorganisms that contain protein such as collagen and also monomeric P4H enzyme can be lysed at a pH of about 12 to create a lysate. In some embodiments, the cells can be lysed at a pH of about 7, about 8, about 9, about 10, about 11, about 12, about 13 or higher. In some embodiments, the pH of the lysate can then be lowered to about 7.5. In certain embodiments, the pH can lowered to about 10, about 9, about 8, about 7.5, about 7, about 6, or about 5. In particular embodiments, reaction components, including one or more of ascorbic acid, sodium ascorbate, DTT, or an iron (II) species (such as FeSO4) can be added to the lysate following pH reduction. In certain embodiments, alpha-Ketoglutarate (AKG or 2-oxoglutarate) can also be added to the reaction.

In certain embodiments, the ex vivo hydroxylation reaction can be performed at a temperature ranging from about 16° C. to about 40° C., for example about 32° C. In some embodiments, the hydroxylation reaction can be performed at about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C. or about 40° C. In some embodiments, the ex vivo hydroxylation reaction can take place over about 30 mins to about 5 hours, for example about 3 hours. In some embodiments, the ex vivo hydroxylation can take place over about 30 minutes, about 45 minutes, about 1 hour, about 1.5 hours, about 2 hours, about 2.5 hours, about 3 hours, about 3.5 hours, about 4 hours, about 4.5 hours, or about 5 hours.

Once the ex vivo hydroxylation reaction is complete, the monomeric P4H can be inactivated by adding an acid to lower the pH of the solution to 4 or adding 50% - 80% methanol by volume.

In an alternative embodiment, the DNA sequence of the monomeric P4H enzyme can be transfected into a microorganism and utilized to hydroxylate proteins intracellularly/in vivo. In some embodiments, the microorganism can also express a protein to be hydroxylated. In some embodiments, the microorganism can express collagen as the protein to be hydroxylated.

In typical embodiments, the transfected microorganism can be grown in media appropriate for the particular microorganism under conditions well known to one of ordinary skill in the art. In some embodiments, suitable media for the reaction can be, for example, LB (Lysogeny broth) for E.coli, BMGY (Buffered Glycerol-complex Medium) for Pichia, YPD (yeast extract peptone dextrose) for Pichia, or HMP (Sodium hexametaphosphate) for Pichia. The temperature of the media can range from about 16° C. to about 42° C. In some embodiments, the temperature of the media can be about 16° C., about 18° C., about 20° C., about 22° C., about 24° C., about 26° C., about 28° C., about 29° C., about 30° C., about 31° C., about 32° C., about 33° C., about 34° C., about 35° C., about 36° C., about 37° C., about 38° C., about 39° C., about 40° C., about 41° C., or about 42° C.

In some embodiments, the transfected microorganism can be Pichia, and the temperature of the media can range from about 28° C. to about 36° C., for example about 32° C. In some embodiments, the temperature of the media can be about 28° C., about 29° C., about 30° C., about 31° C., about 32° C., about 33° C., about 34° C., about 35° C. or about 36° C.

In some embodiments, the transfected microorganism can be grown for a time ranging from about 50 hours to about 72 hours, for example about 68 hours. In some embodiments, the microorganism can be grown for about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours. In certain embodiments, co-factors for hydroxylation reaction can include: alpha-Ketoglutarate (AKG or 2-oxoglutarate) and /or molecular oxygen. In embodiments, the substrate for the hydroxylation reaction is molecular collagen.

In some embodiments, the DNA sequence for the monomeric P4H enzyme can be placed in a vector along with: a DNA sequence for a promotor; a DNA sequence for a terminator; a DNA sequence for a selection marker, a DNA sequence for a promoter for the selection marker; a DNA sequence for a terminator for the selection marker; a DNA sequence for a replication origin selected from one for bacteria and one for yeast; and/or a DNA sequence containing homology to the yeast genome (optional to improve efficiency when transformed into a yeast). In some embodiments, the vector can be inserted into (or episomal to) an organism. In some embodiments, the vector then can be transformed into the organism by methods known in the art such as electroporation. In certain embodiments, the organism can be a microorganism. In some embodiments, the vector can also possess a DNA sequence for a secretion signal.

In some embodiments, the DNA of the recombinant P4H enzyme can be transformed into a microorganism along with DNA encoding a protein to be hydroxylated. In some embodiments, the DNA sequence for the monomeric P4H enzyme can be placed in a first vector along with: a DNA sequence for a promoter for the monomeric P4H sequence; a DNA terminator sequence for the monomeric P4H sequence; a DNA sequence for a selection marker; a DNA sequence for a promoter for the selection marker; a DNA sequence for a terminator for the selection marker; a DNA sequence for a replication origin selected from one for bacteria and one for yeast; and/or a DNA sequence containing homology to the host microorganism’s genome. In some embodiments, the DNA sequence for the protein to be hydroxylated can be placed on a second vector along with: a DNA sequence for a promoter for the protein to be hydroxylated; a DNA sequence for a terminator for the protein to be hydroxylated; a DNA sequence for a selection marker; a DNA sequence for a promoter for the selection marker; a DNA sequence for a terminator for the selection marker; a DNA sequence for a replication origin selected from one for bacteria and one for yeast; and/or a DNA sequence containing homology to the host organism’s genome. In some embodiments, the two vectors can then be transformed into the microorganism by methods known in the art such as electroporation. In some embodiments, any vector disclosed herein can also include a DNA sequence for a secretion signal.

Alternatively, in some embodiments, an all-in-one vector can be used, wherein the DNA for the monomeric P4H enzyme, including a promoter and a terminator for the monomeric P4H enzyme sequence; the DNA for the protein to be hydroxylated, including a promoter and a terminator for the sequence of the protein to be hydroxylated; a DNA for a selection marker, including a promoter and a terminator for the selection marker; and/or DNAs with homology to the organism’s genome for integration into the genome are included in the all-in-one vector. The all-in-one vector then can be transformed into the microorganism by methods known in the art such as electroporation.

Suitable promoters for use in the present disclosure include, but are not limited to, AOX1 methanol induced promoter, pDF de-repressed promoter, pCAT de-repressed promoter, Das1-Das2 methanol induced bi-directional promoter, pHTXl constitutive Bi-directional promoter, pGCW14-pGAP1 constitutive Bi-directional promoter and combinations thereof.

The monomeric P4H enzyme described herein can be useful for personal care compositions suitable for application to the skin. The monomeric P4H enzyme can be included in the personal care compostion at a particular purity level. For example, and in some embodiments, the monomeric P4H enzyme can be added as isolated or purified monomeric P4H enzyme (i.e. without any impurities). Alternatively, the monomeric P4H enzyme can be added in lower purity, (e.g., about 25% purified, about 50% purified, about 65% purified, about 75% purified, about 85% purified, about 90% purified, about 95% purified, about 96% purified, about 97% purified, about 98% purified, or about 99% purified by weight). In some embodiments, the amount of monomeric P4H is quanitified by qSDS. In other words, the monomeric P4H enzyme can be added to a personal care product as a purified protein or it can be added as part of the fraction from which the protein is found. In certain embodiments, the monomeric P4H enzyme can be formulated into a cream, a lotion, an ointment, a gel, a serum, or other type of formulation suitable for topical application to the skin of a subject in need thereof.

In some embodiments, the composition can further include a cosmetically-acceptable carrier. The cosmetically-acceptable carrier can comprise from about 50% to about 99%, by weight, of the composition (e.g., from about 80% to about 95%, by weight, of the composition). In some embodiments, the carrier can be about 50%, about 55%, about 60%, about 65%, about 70%, about 75%, about 80%, about 85%, about 90%, about 95%, about 96%, about 97%, about 98%, or about 99%, by weight, of the composition.

The compositions can be use in a wide variety of product types that include but are not limited to liquid compositions such as lotions, creams, gels, sticks, sprays, shaving creams, ointments, cleansing liquid washes and solid bars, pastes, powders, mousses, masks, peels, make-ups, and wipes. These product types can comprise several types of cosmetically acceptable carriers including, but not limited to solutions, emulsions (e.g., microemulsions and nanoemulsions), gels, solids and liposomes).

In some embodiments, the topical compositions described herein can be formulated as solutions. Solutions typically include an aqueous solvent (e.g., from about 50% by weight to about 99% by weight or from about 90% by weight to about 95% by weight of a cosmetically acceptable aqueous solvent). In some embodiments, the solution can be about 50%, about 55%, about 60%, about 65%, about 70%, about 75%, about 80%, about 85%, about 90%, about 95%, about 96%, about 97%, about 98%, or about 99 % by weight of a cosmetically acceptable aqueous solvent. In certain embodiments, the aqueous solvent can be water. In other embodiments, the aqueous solvent can be a mixture of water and one more water-soluble solvents, such as ethanol, isopropanol, glycerol, and the like.

In some embodiments, the topical compositions can be formulated as a solution comprising one or more emollients. Such compositions can contain from about 2% to about 50% by weight of the one or more emollients. In some embodiments, the composition comprises about 2%, about 3%, about 4%, about 5%, about 6%, about 7%, about 8%, about 9%, about 10%, about 12%, about 15%, about 20%, about 25%, about 30%, about 35%, about 40%, about 45%, or about 50% by weight of the one or more emollients. As used herein, “emollients” refer to materials used for the prevention or relief of dryness, as well as for the protection of the skin. A wide variety of suitable emollients are known and can be useful in the personal care compositions. See International Cosmetic Ingredient Dictionary and Handbook, eds. Wenninger and McEwen, (The Cosmetic, Toiletry, and Fragrance Assoc., Washington, D.C., 7.sup.th Edition, 1997) (hereinafter “CTFA Handbook”) which contains numerous examples of suitable materials.

In some embodiments, the composition can be a lotion. In some embodiments, the lotion comprises from about 1% to about 20% by weight (e.g., from about 5% n to about 10% by weight) of one or more emollients and from about 50% n to about 90% by weight (e.g., from about 60% by weight to about 80% by weight) water. In some embodiments, the lotion can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of one or more emollients. In some embodiments, the lotion can comprise about 50%, about 55%, about 60%, about 65%, about 70%, about 75%, or about 80% by weight water.

In yet another embodiment, the composition can be a cream. In certain embodiments, a cream typically comprises from about 5% to about 50% by weight (e.g., from about 10% by weight to about 20% by weight) of one or more emollients and from about 45% by weight to about 85% by weight (e.g., from about 50% by weight to about 75% by weight) water. In some embodiments, the cream can comprise about 5%, about 6%, about 7%, about 8%, about 9%, about 10% about 15%, about 20%, about 25%, about 30%, about 35%, about 40%, about 45%, or about 50% by weight of one or more emollients. In some embodiments, the cream can comprise about 45%, about 50%, about 55%, about 60%, about 65%, about 70%, about 75%, about 80%, or about 85% by weight water.

In still another embodiment, the composition can be an ointment. In certain embodiments, the ointment can comprise a base of comprising one or more animal or vegetable oils or one or more semi-solid hydrocarbons. In certain embodiments, the ointment can comprise from about 2% by weight to about 10% by weight of an emollieiit(s) plus from about 0.1% by weight to about 2% by weight of one or more thickening agents. In some embodiments, the ointment can comprise about 2%, about 3%, about 4%, about 5%, about 6%, about 7%, about 8%, about 9% or about 10% by weight of one or more emollients. In some embodiments, the ointment can comprsie about 0.1%, about 0.2%, about 0.3%), about 0.4%, about 0.6%, about 0.8%, about 1.0%, about 1.2%, about 1.4%, about 1.6%, about 1.8% or about 2.0% by weight of one or more thickening agents. Suitable thickening agents are known to those of ordinary skill in the art as set forth in the CTFA Handbook.

In some embodiments, the composition can be an emulsion. If the carrier is an emulsion, from about 1% to about 10% by weight (e.g., from about 2% to about 5% by weight) of the carrier can comprise an emulsifier(s). In some embodiments, about 1%, about 2%, about 3%, about 4%, about 5%, about 6%, about 7%, about 8%, about 9%, or about 10% by weight of the carrier can comprise an emulsifier(s). Emulsifiers can be nonionic, anionic or cationic.

In some embodiments, the lotions pr creams can be formulated as emulsions. Typically, such lotions can comprise from 0.5% to about 5% by weight of an emulsifier(s). Such creams would typically comprise from about 1% to about 20% by weight (e.g., from about 5% to about 10% by weight) of an emollient(s); from about 20% to about 80% by weight (e.g., from 30% to about 70% by weight) of water; and from about 1 % to about 10% by weight (e.g., from about 2% to about 5% by weight) of an emulsifier(s).

Single emulsion skin care compositions, such as lotions and creams, of the oil-in-water type and water-in-oil type are well-known in the cosmetic art and are useful for the personal care compositions. Multiphase emulsion compositions, such as the water-in-oil-in-water type are also useful. In general, such single or multiphase emulsions contain water, emollients, and emulsifiers as essential ingredients.

The personal care compositions of this disclosure can also be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents. In some embodiments, the gel comprises about 0.1%, about 0.2%, about 0.3%, about 0.4%, about 0.5%, about 1.0%, about 1.5%, about 2.0%, about 2.5%, about 3.0%, about 3.5%, about 4.0%, about 4.5%, or about 5.0% by weight, of such gelling agents.

The personal care compositions useful in the subject disclosure can contain, in addition to the aforementioned components, a wide variety of additional oil-soluble materials and/or water-soluble materials conventionally used in compositions for use on the skin at their art-established levels.

The personal care compositions can be applied to or on skin as needed and/or as part of a regular regimen ranging from application once a week up to one or more times a day (e.g., twice a day). The amount used will vary with the age and physical condition of the end user, the duration of the treatment, the specific compound, product, or composition employed, the particular cosmetically-acceptable earner utilized, and like factors.

The monomeric P4H enzyme described herein can be useful for skin care benefits in personal care applications such as anti-wrinkle, improved skin pigmentation, hydration, reduction of acne, prevention of acne, reduction of black heads, prevention of blackheads, reduction of stretch marks, prevention of stretch marks, prevention of cellulite, reduction of cellulite and the like. By improved skin pigmentation is meant either evening out skin pigmentation or reducing skin pigmentation to provide fair skin.

The monomeric P4H enzyme described herein can also be combined with other skin care benefit ingredients such as, but not limited to salicylic acid, retinol, benzoyl peroxide, vitamin C, glycerin, alpha-hydroxy acids, hydroquinone, kojic acid, hyaluronic acid and the like.

In the context of the present description, all publications, patent applications, patents and other references mentioned herein, if not otherwise indicated, are explicitly incorporated by reference herein in their entirety for all purposes as if fully set forth, and shall be considered part of the present disclosure in their entirety.

Unless otherwise defined, all technical and scientific terms used herein have the same meaning as commonly understood by one of ordinary skill in the art to which this disclosure belongs. In case of conflict, the present specification, including definitions, will control.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

Further, unless otherwise explicitly stated to the contrary, when one or multiple ranges or lists of items are provided, this is to be understood as explicitly disclosing any single stated value or item in such range or list, and any combination thereof with any other individual value or item in the same or any other list.

The examples are illustrative, but not limiting, of the present disclosure. Other suitable modifications and adaptations of the variety of conditions and parameters normally encountered in the field, and which would be apparent to those skilled in the art, are within the spirit and scope of the disclosure.

It is to be understood that the phraseology or terminology used herein is for the purpose of description and not of limitation. The breadth and scope of the present disclosure should not be limited by any of the above-described exemplary embodiments, but should be defined in accordance with the following claims and their equivalents,

### EXAMPLES

### Example 1: Over-Expression of Mimi-Virus P4H in E.Coli

### Primers Used

For N terminal His tag:

Forward (SEQ ID NO: 15)

GAGCTCGGTACCATGCACCACCACCACCACCACGTGCTGTCAAAGTCCTGTGT
CAGTCAC

Reverse (SEQ ID NO: 16):

AAGCTTGAATTCTTAGGAGAACTTACGCTCACGAAACCACA

For C terminal His Tag: Forward (SEQ ID NO: 17):

GAGCTCGGTACCATGGTGCTGTCAAAGTCCTGTGTCAGTC

Reverse (SEQ ID NO: 18):

AAGCTTGAATTCTTAGTGGTGGTGGTGGTGGTGGGAGAACTTACGCTCACGA
AACCAC

gBlock was ordered from IDT and gene was amplified using standard PCR conditions.

### Polymerase Chain Reaction Conditions

The reaction mix components are as follows: pfu polymerase buffer 1x, 0.2 mM dNTPs each, 0.5 µM forward primer, 0.5 µM reverse primer, 0.02 U/µL pfu polymerase and 10 ng/mL gBlock. The thermal cycler was programmed as follows:
1. 95° C.-60 seconds
2. 95° C. -30 seconds
3. 56° C. -45 seconds
4. 72° C. - 30 seconds
5. 72° C. -7 minutes
25 repeat cycles from #2 to #4.

The amplified gene was cut with restriction enzymes EcoR I and Kpn I. The digested DNA was cleaned by agarose gel extraction using commercial kit before ligation into pCOLDIII vector. Ligation was set-up with a molar ratio of 1:3 (plasmid: insert) in 10 µL reaction mix. Typically, a ligase reaction mix had 3 ng/L digested plasmid vector, 9 ng/mL of the insert, 1 µL 10 X ligase buffer and 1 U/mL ligase. Ligation reaction mix was transformed into E. coli DH5a cells. Cells were spread on LB Ampicillin plates (6.25 g LB powder mix, 4 g agar, 250 mL DDI water, 0.1 mg/mL Ampicillin) before recovering in SOC medium for 1 hour at 37° C. Plates were incubated at 37° C. overnight; individual colonies that appeared next day were tested for gene fragments by colony PCR. Clones that showed amplification for desired fragments were inoculated on LB broth having 0.10 mg/mL ampicillin and grown overnight at 37° C., 250 rpm. Recombinant plasmid from these overnight grown cultures were isolated using kit from Zymergen and given for sequencing. Plasmid sequencing was done at Eueofin Inc. sequencing facility and gene specific primers were used for sequencing reactions.

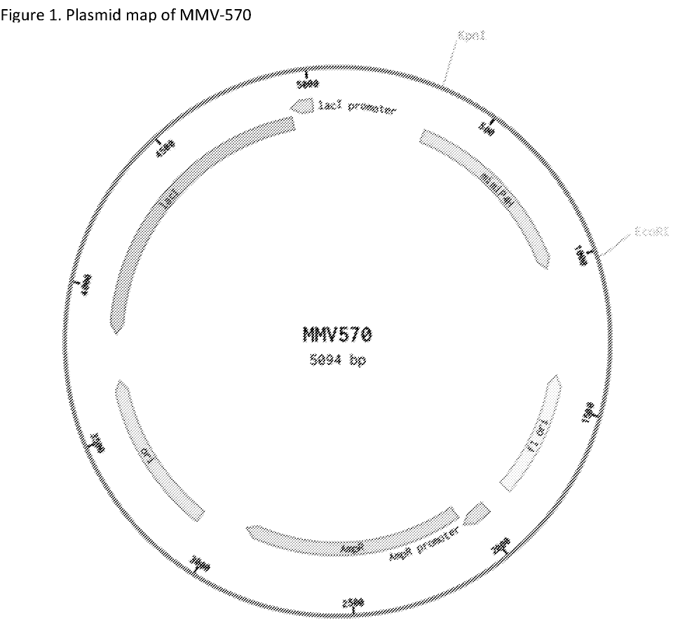

Confirmed plasmids (FIG. 1) were transformed into chemically competent E. coli BL21 (DE3) cells using heat shock method. Transformants were allowed to recover in SOC medium (37° C., 50 min), then plated onto LB Ampicillin agar plates and incubated at 37° C. for 16 hours. Several colonies appeared on overnight-incubated plates; a single colony from this plate was inoculated in 5 mL LB medium having antibiotic with the same concentrations as above. The culture was incubated overnight at 37° C. with constant shaking at 250 rpm. On the following day, 5 mL of the overnight cultures was used to inoculate 500 mL of fresh LB media having the same antibiotics, in 3 L Erlenmeyer flask. The culture was incubated at 37° C., 250 rpm, and protein expression was induced by adding 1mM IPTG when OD600 reached 0.8. The induced culture was moved to 18° C. and allowed to grow for 12 hours. Cells were harvested by centrifugation at 4° C., 3000 x g for 20 minutes. 20 g cell pellets were re-suspended in 20 ml lysis buffer (xTractor buffer from Takara bio) and incubated for 30 minutes at room temperature with constant mixing. Lysed culture was clarified at 12000 x g, 4° C. for 30 minutes and supernatant thus obtained were loaded on equilibrated Ni-NTA columns.

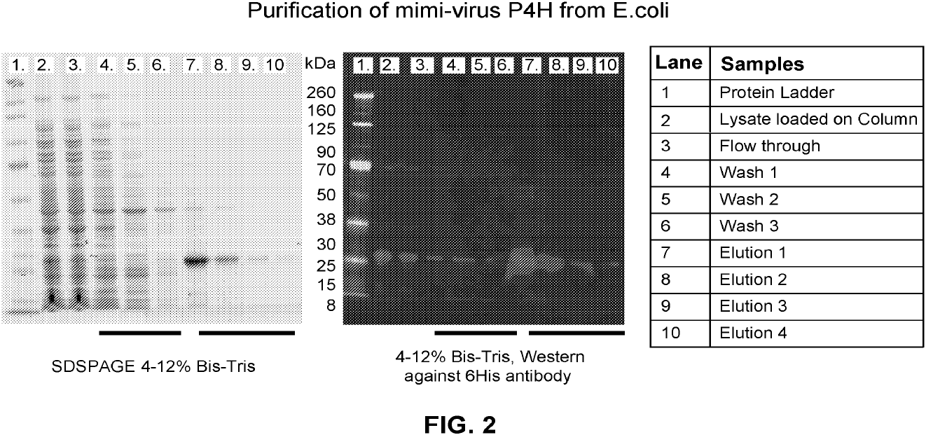

5 ml Ni-NTA (10 ml of 50% solution) beads were washed with 2 X volume of water and then with 5 X volume of lysis buffer (25 mM Tris pH 7.5, 50 mM NaCl and 20 mM Imidazole). Clarified lysate and Ni-NTA beads (equilibrated with lysis buffer as above) were mixed for 1 hour. This mix was poured into centrifuge columns and centrifuged at 1000 X g for 1-2 minutes at 4° C. About 2.5 ml beads should be there in 2 purification columns to get original volume of total 5 ml. The flow through was stored to check for any protein loss during the binding step. Beads that were collected in the centrifuge columns were washed with 50 ml of wash buffer (25 mM Tris pH 7.5, 50 mM NaCl and 50 mM Imidazole) sequentially, adding 10 ml at a time, centrifuging for 1000 X g for 1-2 minutes 4° C. Washings were also collected to check for the loss of mVP4H (Mimivirus P4H) during the washing step. 6 elution fractions were collected from each of the purification columns by passing 2.5 ml of elution buffer (25 mM Tris pH 7.5, 50 mM NaCl and 300 mM Imidazole) each time and centrifuge at 1000 rpm for 1-2 minutes at 4° C. Centrifuge elution fractions at 14000 X g for 5 minutes to remove any insoluble debris. Flow through, washings and all the fractions were checked on SDS PAGE (FIG. 2). Elution fractions were pooled and concentrated down to ~ 10 ml using 10 MW cut off protein concentrator. Concentrated purified mVP4H put for dialysis overnight at 4° C. in ~ 1 liters of 50 mM Tris-HCl pH 7.5, 100 mM NaCl buffer using 10 kDa cut off dialysis tubing in the cold room. One buffer change done next day for at least 3 hours under the cold condition (4° C.) and then dialyzed protein was taken out from dialysis tubes, centrifuge at 14000 X g for 10 minutes to remove any insoluble/aggregated protein. Q-bit protein estimation done on purified protein (at least 50 times diluted). Purified protein stored in several 500 ul aliquots at -80° C.

### Example 2: Over-Expression of Intracellular Mimi-Virus P4H in Pichia

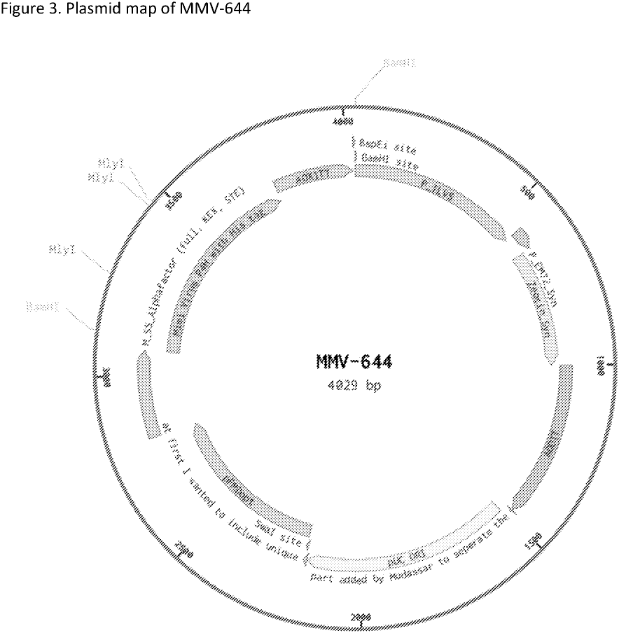

The DNA sequence of monomeric prolyl 4-hydroxylase was acquired from IDT. Polymerase chain reactions were done using the DNA sequences as templates with primers MM-0579 (SEQ ID NO: 10); MM-0580 (SEQ ID NO: 20); MM-1569 (SEQ ID NO: 21), MM-1570 (SEQ ID NO: 22); MM-0784 (SEQ ID NO: 23) and Gibson assembled into vector MMV-644 (SEQ ID NO: 12). The final vector MMV-644 (FIG. 3) was confirmed by sequencing and transformed into Pichia pastoris yeast strain PP97 to generate strain PP765.

### Polymerase Chain Reaction for Pichia

Reaction mix: pfu polymerase buffer 1 x, 0.2 mM dNTPs each, 0.5 µM forward primer, 0.5 µM reverse primer, 0.02 U/µL pfu polymerase and 10 ng/mL gBlock.

Thermal cycler was programmed as:
1. 95° C.-60 seconds
2. 95° C.-30 seconds
3. 56° C.-45 seconds
4. 72° C.- 30 seconds
5. 72° C.-7 minutes
repeat 25 cycles from #2 to #4

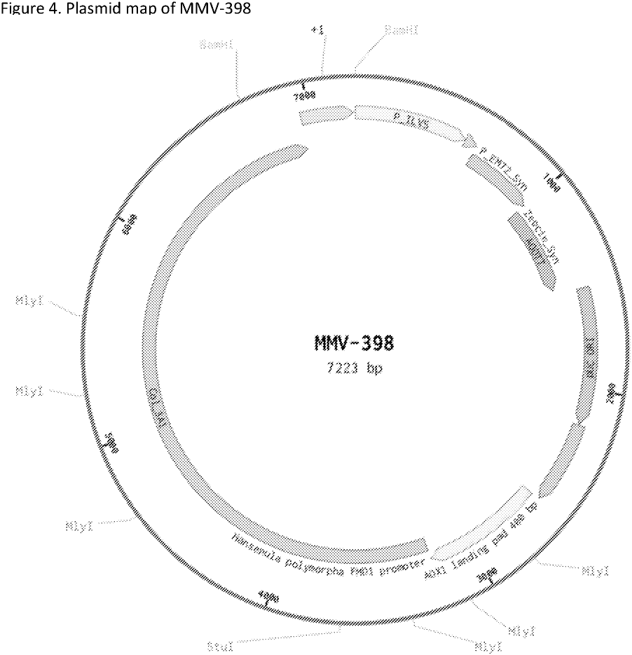

PP421 was generated by digesting MMV-398 (FIG. 4) with Pme I and transforming into PP97. PP153 contains the collagen driven by pDF promoter.

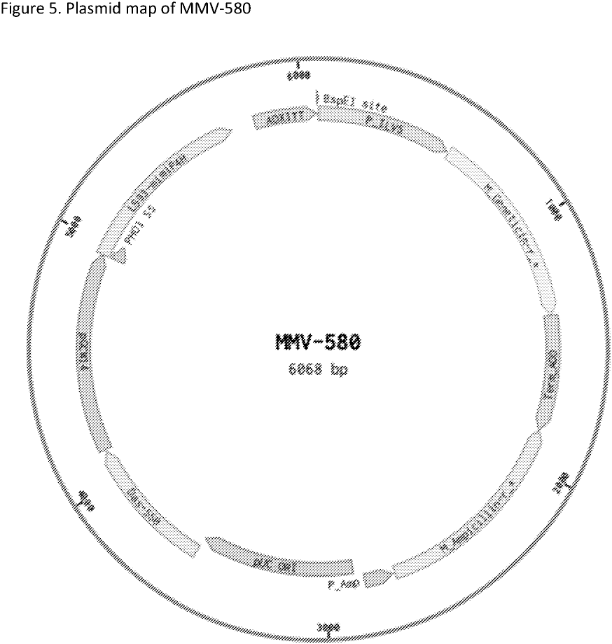

PP654 was generated by digesting MMV-580 (FIG. 5) with Pme I and transforming into PP421.

PP657 was generated by digesting MMV-580 (FIG. 5) with Pme I and transforming into PP97.

1. Ni-NTA purification: 5 ml Ni-NTA (10 ml of 50% solution) beads were washed with 2 X volume of water and then with 5 X volume of lysis buffer (25 mM Tris pH 7.5, 50 mM NaCl and 20 mM Imidazole). pH of the 20 ml media was adjusted to 7.5 using 2N NaOH for the secreted mimi P4H. pH adjusted media and Ni-NTA beads (equilibrated with lysis buffer as above) were mixed for 3 hours at 4° C.

For the intracellular mimi P4H, pellets were resuspended in lysis buffer, mixed with beads and lysed using tissulyser. Lysed culture was clarified at 12000 x g, 4° C. for 30 minutes and supernatant thus obtained was mixed with beads overnight at 4° C. The steps are common for both secreted and intracellular mimiP4H purification.

The mix was poured into centrifuge columns and centrifuged at 1000 X g for 1-2 minutes at 4° C. About 2.5 ml beads should be there in 2 purification columns to get original volume of total 5 ml. The flow through was stored to check for any P4H loss during the binding step. Beads that were collected in the centrifuge columns were washed with 50 ml of wash buffer (25 mM Tris pH 7.5, 50 mM NaCl and 50 mM Imidazole) sequentially, adding 10 ml at a time, centrifuging for 1000 X g for 1-2 minutes 4° C. Washings were also collected to check for the loss of mVP4H (mimivirus P4H) during the washing step. Elution fractions were collected from each of the purification columns by passing 2.5 ml of elution buffer (25 mM Tris pH 7.5, 50 mM NaCl and 300 mM Imidazole) each time and centrifuge at 1000 rpm for 1-2 minutes at 4 For the intracellular. Centrifuge elution fractions at 14000 X g for 5 minutes to remove any insoluble debris. Flow through, washings and all the fractions were checked on SDSPAGE. Elution fractions were pooled and concentrated down to ~ 10 ml using 10 MW cut off protein concentrator. Concentrated purified mVP4H put for dialysis overnight in ~ 1 liters of 50 mM Tris-HCl pH 7.5, 100 mM NaCl buffer using 10 kDa cut off dialysis tubing in the cold room. One buffer change done next day for at least 3 hours under the cold condition (4° C.) and then dialyzed protein was taken out from dialysis tubes, centrifuge at 14000 X g for 10 minutes to remove any insoluble/aggregated protein. Q-bit protein estimation done on purified protein (at least 50 times diluted). Purified protein stored in several 500 ul aliquots at -80° C.

2. Direct Media Dialysis: For the secreted mimi P4H, fermentation media was directly transferred into dialysis tubing (10 ml, 10 kDa cut off) and put for dialysis overnight in 1 liters of 50 mM Tris-HCl pH 7.5, 100 mM NaCl buffer at 4° C. in the cold room. Two buffer changes were done next day for at least 3 hours each. Dialyzed protein taken out from dialysis tubes, centrifuge at 14000 X g for 10 minutes to remove any insoluble/aggregated protein. Q-bit protein estimation done on purified protein (at least 50 times diluted). Purified protein stored in several 500 ul aliquots at -80° C.

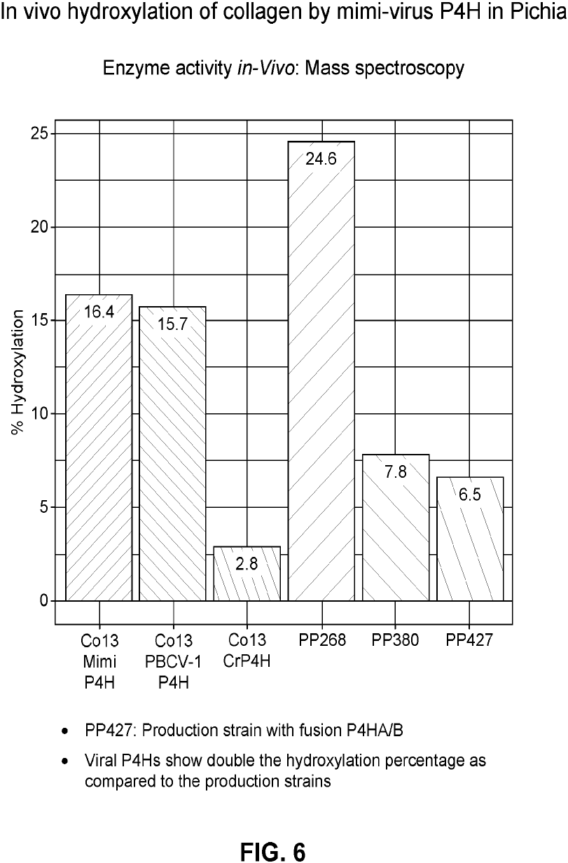

Fermentation grown samples were run on SDS PAGE gel, specific collagen band was cut and sent out for Mass spec analysis. FIG. 6 shows the hydroxylation levels obtained for PP654 when grown in production media in fermenters. MimiP4H was found to be active on full length collagen (with foldON) as it showed -17% hydroxylation.

### Testing Enzyme Activity in Small Scale

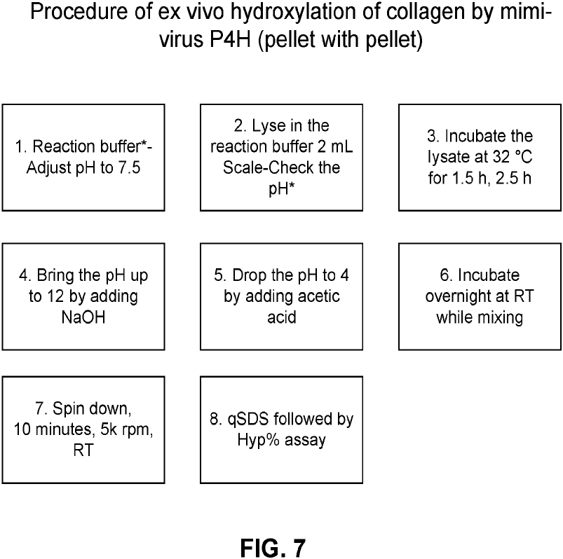

Ex Vivo (Method:1): Step wise method is described in FIG. 7.

Reaction buffer has following components:
5 mM Iron Sulfate (made fresh) - First make 0.05 M stock and then use that to make 5 mM working stock
10 mM DTT (fresh frozen stocks)
0.2 M Ascorbic Acid (made fresh)
1 M Tris-HCl pH 7.5
2-oxoglutarate (0.4 M)

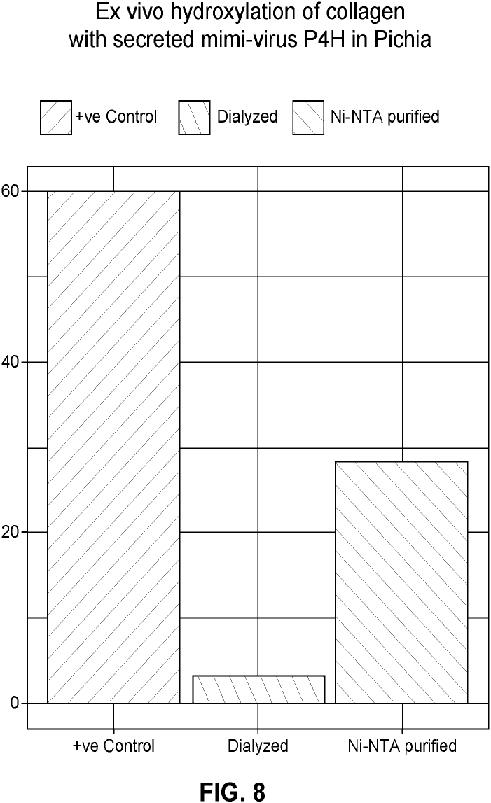

Fermenter grown samples were collected in micro centrifuge tubes, 300 mg of pellets were resuspended in reaction buffer and lysis was performed in 96 well plate. 300 mg cell pellet was resuspended in 2 ml buffer and distributed into 3 different 96 well plate. Cells were lysed in tissue lyser for 15 minutes. The pH of the lysate was checked and adjusted to 7.5 and incubated at 32° C. for 1.5 and 2.5 hours. Later the collagen was purified using our standard high low pH protocol, quantified on qSDS gels (FIG. 8) and used for Hyp% assay.

### Testing Enzyme Activity in Small Scale

Ex Vivo (Method:2, lysate: lysate mixing):

Two different lysates were used in this method ()
Collagen only strain (PP681)
P4H only strains (PP547, PP635, PP657, PP658, PP659)

These strains were grown separately in a shake flask with BMGY media.

The cell pellets (mixed pellets) were combined in 1:10 ratio (0.1 g Col3 strain: 1 g P4H strain) in 10 ml reaction buffer (same steps as in FIG. 7)

The ‘mixed’ pellets were lysed in 10 ml reaction buffer, pH adjusted and incubated for 2 hours at 32° C.

The ‘reaction mix’ was purified for Col3 using high-low pH method.

qSDS followed by Hyp% assay was performed.

### Example 3: Co-Expression of Collagen With Mimi-Virus P4H in Pichia

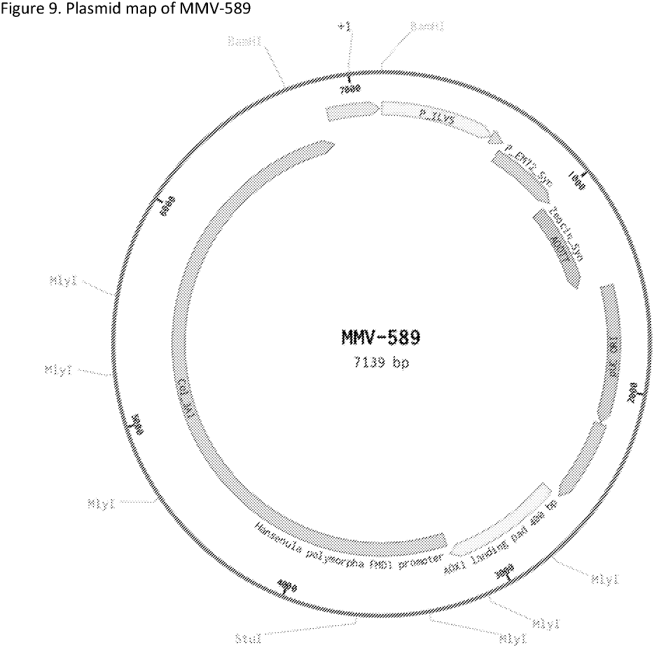

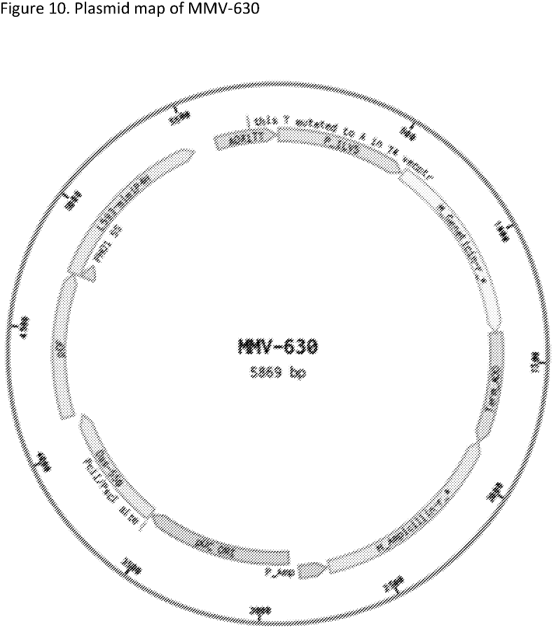

PP681 was generated by digesting MMV-589 (FIG. 9) with Pme I and transforming into PP97. PP735 was generated by digesting MMV-580 (FIG. 5) with Pme I and transforming into PP681. PP758 was generated by digesting MMV-630 (FIG. 10) with Pme I and transforming into PP681.

### Monomeric P4H Activity Testing

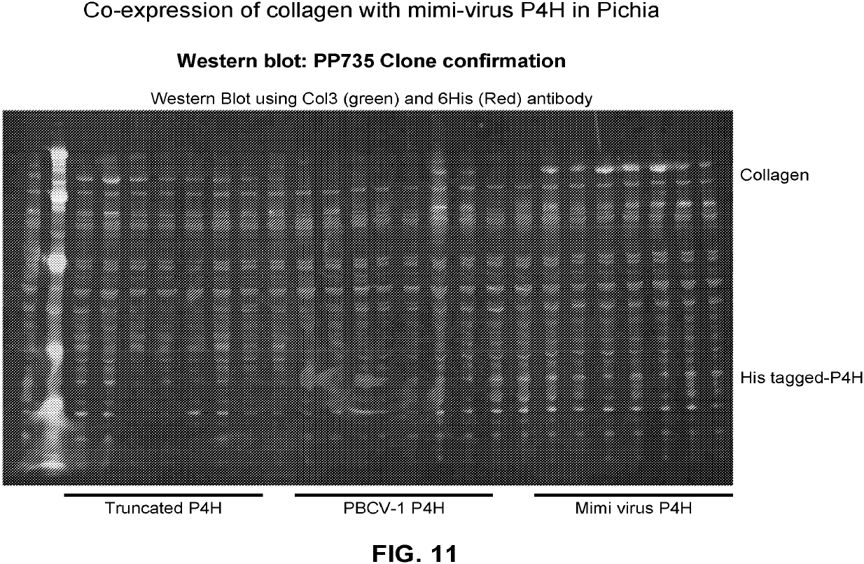

Small P4Hs (including mimiP4H) were transformed into strains that have non FoldON collagen (PP681). Therefore, PP681 background was used. A Western blot was performed to confirm the clones (FIG. 11) and new transformants were named PP735. Four of the transformants that showed mimiP4H bands on western were selected and grown in 50 ml BMGY media in shake flasks and tested for in vivo as well as for ex vivo enzyme activity.

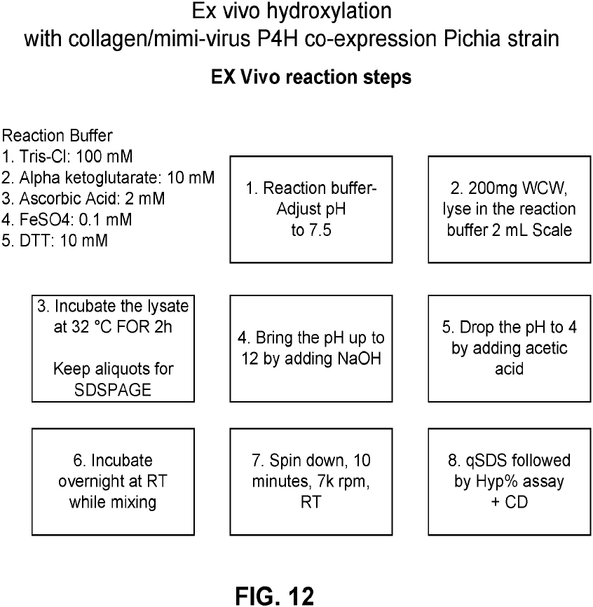

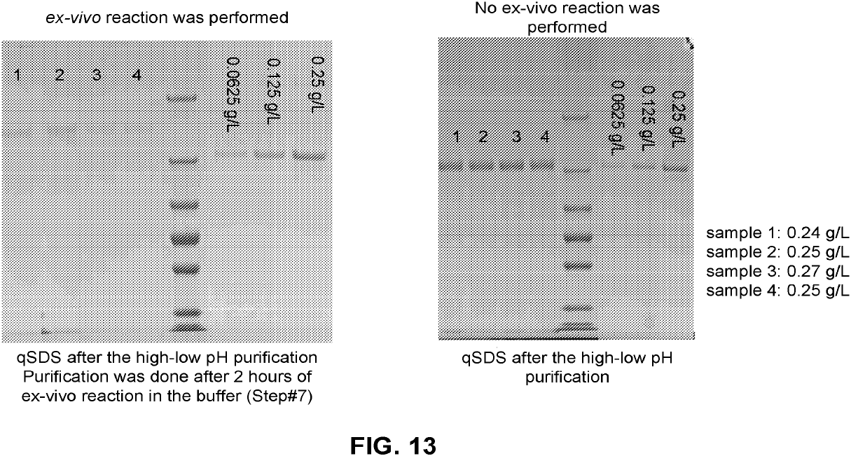

All 4 transformants were tested using the ex vivo steps described in FIG. 12. Control reactions where no reaction components were added were immediately run through high low pH purification. These control reactions represent the in vivo hydroxylation activity of mimiP4H. All the samples were purified using the standard pH change protocol and quantified using qSDS (FIG. 13). Recovery was much higher for the samples that did not undergo ex vivo reaction in the presence of reaction components. N-Pro cleavage was also incomplete for the ex vivo samples.

### Example 4: Secretion of Monomeric P4H in Pichia

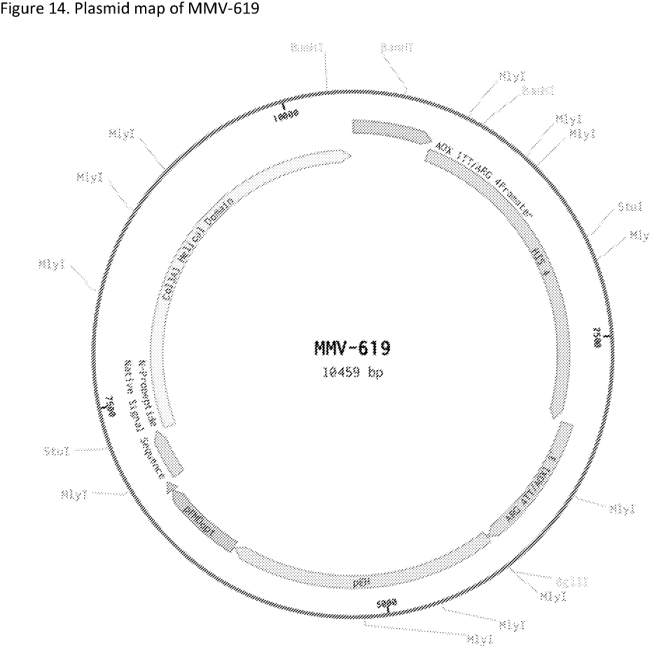

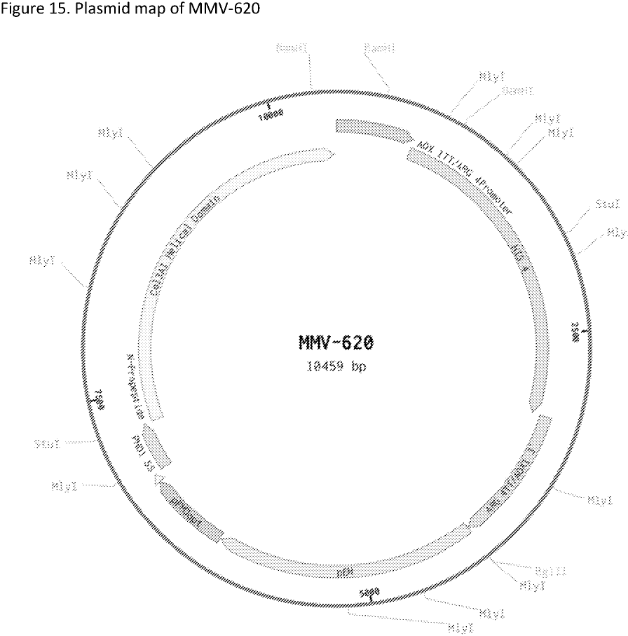

PP765 was generated by digesting MMV-644 (FIG. 3) with Swa I and transforming into PP97. PP765 contains the monomeric prolyl 4-hydroxylase with 6X His tag at the C-terminus driven by pDF promoter and a secretion signal from Saccharomyces cerevisiae alpha mating factor. PP749 was generated by digesting MMV-619 (FIG. 14) with Pme I and transforming into PP480. PP766 was generated by digesting MMV-644 (FIG. 3) with Pme I and transforming into PP749. PP750 was generated by digesting MMV-620 (FIG. 15) with Pme I and transforming into PP480. PP767 was generated by digesting MMV-644 (FIG. 3) with Pme I and transforming into PP750.

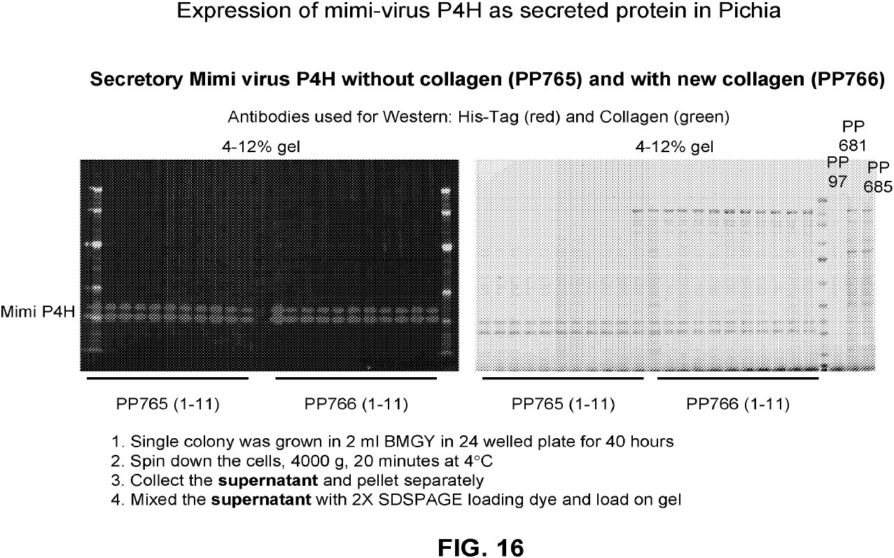

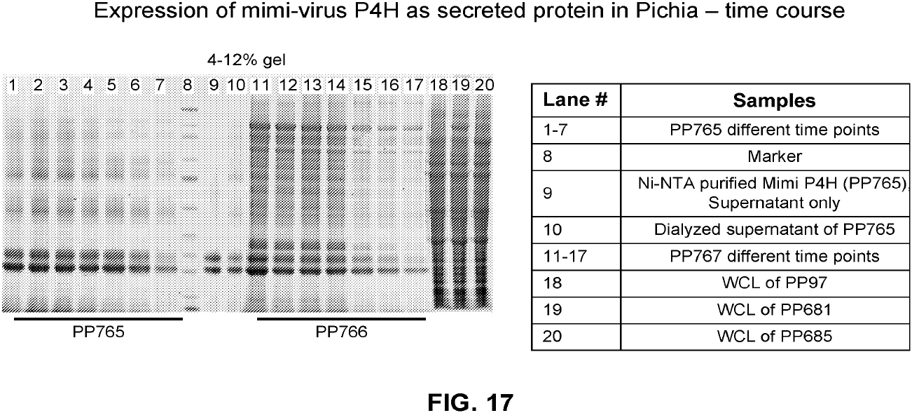

A secretory N terminal signal sequence was introduced in the mimiP4H plasmids (MMV-644) and the plasmids were transformed into His- strains. Different transformants for PP765 (without collagen), PP766 (with native signal sequence collagen) and PP767 (with Pho1 signal sequence collagen) were tested by western blot and on coomassie stained SDS PAGE gels. The transformants were first grown in 24 well plate in BMGY media, later confirmed transformants were also grown in shake flask and fermenters and supernatant was checked in all the cases (FIGS. 16 and 17).

One transformant each for PP765, PP766 and PP767 was grown in 50 ml BMGY media in shake flask and tested in western blot and coomassie stained gels). Most of the mimiP4H was secreted in the media, providing an advantage over intracellular mimiP4H. PP765, PP766 and PP767 were also grown in bioreactors in HMP+peptone media. Different time points of the cultures were collected and analyzed on gel (FIG. 17). The supernatant was purified using Ni-NTA columns as well as by dialyzing the media.

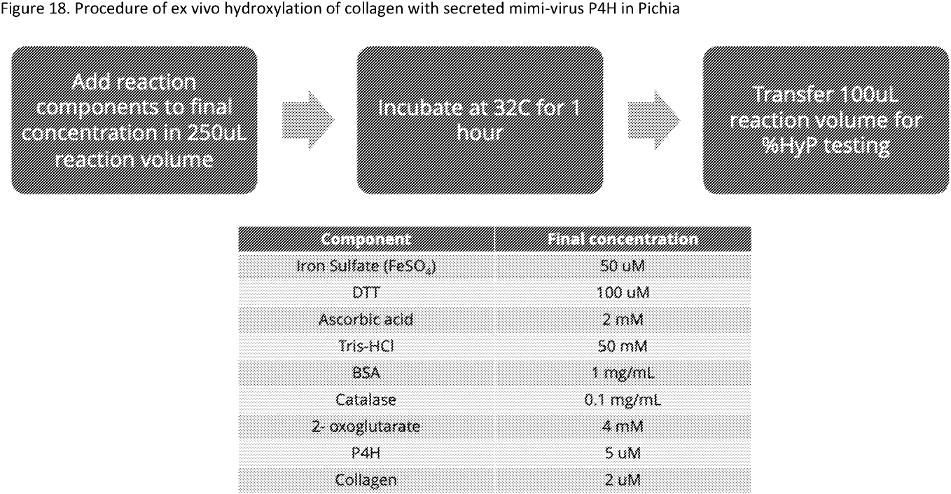

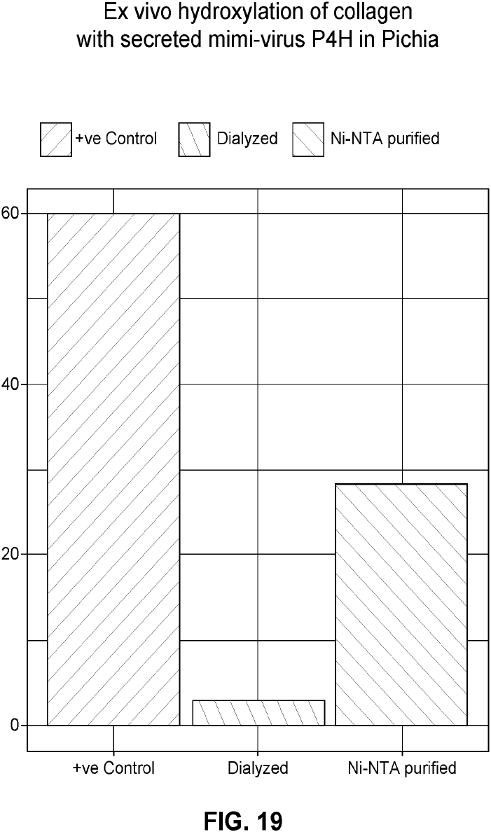

Activity tests: Secreted Mimi P4H from the fermentation supernatant was purified using dialysis and also by Ni-NTA column. Purified P4H was used for the in-vitro hydroxylation reaction was set as described in FIG. 18. %HyP was measured using a colorimetric assay and it was observed that there is an increase in the hydroxylation level of the collagen substrate in comparison to the positive control. Ni-NTA purified mimiP4H showed 24% hydroxylation. However the dialyzed supernatant activity could not be accurately measured due to high background color. A positive control reaction was carried out using the fusion bovine P4H (FIG. 19).

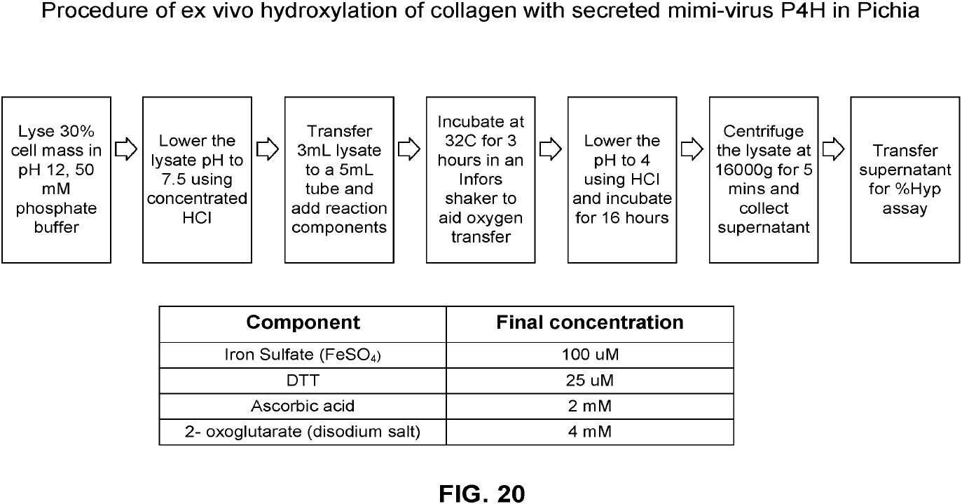

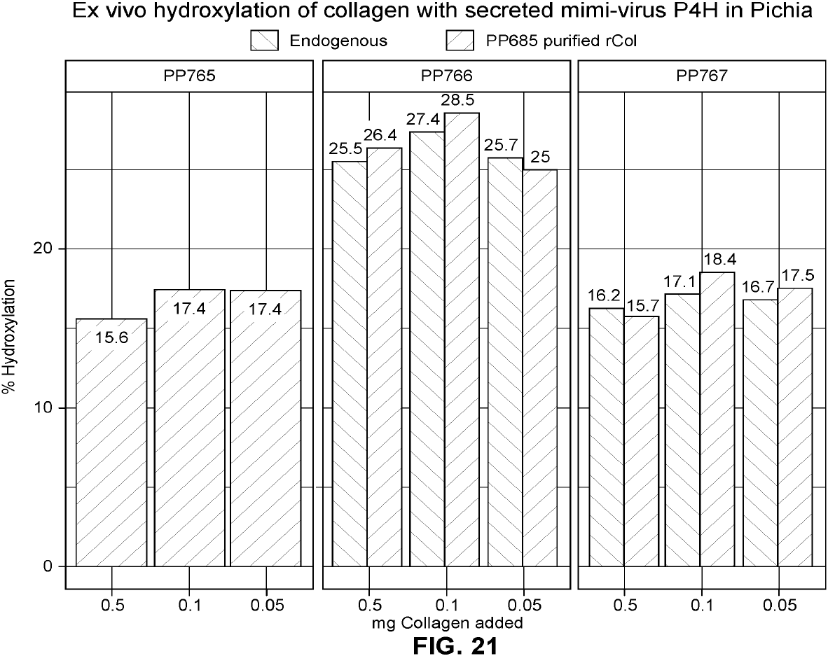

We also demonstrated that mimi virus P4H from fermenter supernatant is active without purification. Fermentation supernatant from three separate Mimi virus P4H secretion strains were collected and 0.05, 0.1 and 0.5 mg of purified collagen were added along with the reaction components (FIG. 20). All reactions showed an increase in the hydroxylation over the pre-reaction levels (~3%). Strains PP766 and PP767 also secrete collagen along with mimiP4H. An increase in hydroxylation was observed for both the secreted collagen and added purified collagen (FIG. 21).

### Example 5: Hydroxylation Assay

The monomeric prolyl 4-hydroxylase enzymatic activity from PP765 was measured by a Hydroxylation assay. Acid hydrolysis of in-vitro hydroxylation reactions containing collagen were mixed with concentrated hydrochloric acid (1:1) and were performed at 125° C. for a minimum of 18 hours. The hydrolysis products were then dried completely and then resuspended with Milli-Q water. The resuspended samples were then centrifuged at 15,0000 rpm for 5 minutes to remove precipitates and debris. A reaction solution, with component final concentrations upon addition to the centrifuged supernatant were the following - 2.67% citric acid (w/v), 3.86% sodium acetate (w/v), 1.87% sodium hydroxide (w/v), 0.64% glacial acetic acid (v/v), 6.7% isopropanol (v/v) and 34 mM Chloramine T. This mixture was incubated at 30° C. for 25 minutes with shaking at 400 rpm. A separate reaction solution, with final concentrations added to the above mixture consisted of 536 mM p-dimethylaminobenzaldehyde (4-DMAB), 12% HC1 (v/v) and 28% isopropanol (v/v) and was incubated for 25 minutes at 65° C. with shaking at 250 rpm. The absorbance was measured immediately at 560 nm using a spectrophotometer. The molecular weight of collagen used and the number of hydroxyproline sites and prolines in the helical region are needed to calculate percent hydroxyproline.

### Example 7

In-vitro hydroxylation in lysate was performed on cells lysed at pH 12 using NaPO4 buffer followed by mixing with 0.1 mM FeSO4, 2 mM ascorbic acid, 25 mM DTT and 25 mM alpha-ketoglutaric acid. The mixture was adjusted to pH 7.5 and incubated for 3 hours at 32° C. by shaking in an incubator for the reaction to proceed. Following completion of the reaction, the pH was dropped to 4 and the reaction was mixed overnight (~ 18 hours) at 25° C. and centrifuged at ~ 7,000 xg to harvest the supernatant. The supernatant was dialyzed against water or buffer and used in the hydroxyproline assay.

### Example 8: Ex Vivo Reaction Condition

### Generating Ferm-Sup

Freshly harvested fermentation broth, consisting of media and cells, is spun at 17,000 xg for 5 minutes to create a cell pellet and supernatant. This supernatant is poured off and called ferm-sup, it can now be frozen.

### Ex Vivo Reaction

The ferm-sup is thawed if frozen, and 750 uL aliquoted into 1.5 mL microcentrifuge tubes. Reaction components are added to the tubes to a final concentration of 25 mM Alpha-ketoglutarate, 25 mM DTT, 2 mM Ascorbate, 0.1 mM Iron Sulfate. Purified collagen is then added to the tubes, in the experiment 500 ug, 100 ug, and 50ug were added from the same stock. The tubes are then placed into the heat block of a thermomixer at 32C and left shaking at 3000 rpm for 3 hours. After the reaction the samples are run on SDS-PAGE gel and the bands corresponding the collagen cut out and sent for Liquid Chromatography Mass-Spec to determine their hydroxylation state. Since pp766 and pp767 excrete their own collagen and it has not been cleaved during the purification process, it runs slightly higher than the spiked in collagen. These are represented as “endogenous”, meaning to the ferm-sup and strain, and “PP685” the strain which we derived the purified collagen from. The reported hydroxylation state of PP685 collagen before the reaction is 4%.

### Example 9: Mass Spec Based Hydroxylation Measurement

A sample solution which contains at least 50 µg of protein to 200 µl with 100 mM Tris-HCl, pH 8.5 is used 55 µg of Abcam recombinant Human Collagen (Abcam, catalog # ab73160,) is used as the positive control. 800 µL of methanol is added to the sample, mixed and stored at -80° C. overnight. The samples are spun at 21,000 xg for 30 min at 4° C. The supernatant is aspirated and, 5-10 µl is left in the tube so as not to disturb the pellet. The pellet is washed twice with 500 µl of cold acetone (100% (v/v) each time. After each wash, it is spun at 21,000 xg for 10 min. The pellet is air dried under hood for 20 to 25 min. If the samples are not dry after 25 min, they are left in the hood until they are dry. To the air-dried pellet, 30 µL of 100 mM Tris-HCl, pH 8.5, 8 M Urea (Sigma, catalog # U5128) is added, and gently mixed to resuspend. If the sample is not totally dissolved, it is spun at 21,000 xg for 15 min. 1.5 µL of 100 mM TCEP (Sigma, catalog # 68957) solution is added to the sample. The sample is incubated at room temperature for 30 min in the dark. 0.6 µL of 500 mM chloroacetamide is added (Sigma catalog # C0267) to the sample. The sample is incubated in the dark at room temperature for another 30 min. 90 µL of 100 mM Tris-HCl, pH 8.5 is added to the sample. 0.6 µL of 500 mM CaC12 is added to the sample. To each sample, 10 µL Trypsin (Promega catalog # V5111) at 0.1 ug/uL is added. The samples are incubated at 37° C. for 18 hours in thethermomixer at 900 RPM. 8 µL of formic acid is added to quench the digestion reaction. 100 µL of sample is tranferred to a mass-spectrometry vial.The samples are tested by Agilent LC-QTOF system (LC: Agilent 1290 Infinity II, MS: Agilent 6545XT). The samples are first separated by an Agilent Peptide Mapping Column held at 50° C. Pure water with 0.1% formic acid is used as mobile phase A while acetonitrile/water (95%/5%, v/v) is used as mobile phase B. The sample is measured in positive mode with Auto MS/MS function. 8 max precursors per cycle. The acquired data is processed by BioConfirm software (Agilent) where the data is searched against predefine collagen sequence. The result in Bioconfirm is exported as a .csv file and then processed by an in-house python script to calculate the Proline Hydroxylation%. For every proline detected in the experiment, the script sums up the peak area of its hydroxylated version (SUMHyP)and non-hydroxylated version (SUMnonHyP), respectively. For each proline, its own Hydroxylation% = SUMHyP/ (SUMHyP + SUMnonHyP). At last, the average Hydroxylation% of all the detected Proline is reported.

### SEQUENCES

SEQ ID NO 1: Mimivirus P4H codon optimized nucleotide sequence for E. coli:

ATGGTGCTGTCAAAGTCCTGTGTCAGTCACTTTAGAAATGTTGGATCCTTGAATAGTAGGGATGTCAATCTGAAAGAT
GACTTTTCCTATGCTAATATTGATGATCCCTATAACAAGCCTTTCGTCCTAAATAACCTAATAAACCCTACCAAGTGT
CAAGAGATCATGCAATTTGCCAATGGCAAGTTGTTTGACTCCCAAGTCCTGAGTGGCACGGACAAGAACATACGTAAC
TCTCAACAAATGTGGATATCCAAGAACAACCCTATGGTAAAACCCATTTTCGAGAACATATGCAGGCAGTTTAACGTA
CCCTTTGATAATGCCGAGGACCTACAGGTCGTCCGTTACTTGCCTAATCAATATTATAATGAGCATCATGACTCATGC
TGTGACTCCTCCAAGCAATGCAGTGAATTTATAGAGAGGGGCGGTCAGAGGATTCTGACCGTTTTAATTTACCTAAAC
AACGAGTTCTCAGATGGACACACGTACTTTCCTAATTTAAACCAAAAGTTCAAGCCCAAGACTGGTGATGCTTTGGTT
TTTTACCCTTTAGCCAACAACTCTAATAAATGTCACCCATACAGTCTACACGCAGGTATGCCCGTCACGTCAGGAGAG
AAGTGGATTGCTAATCTGTGGTTTCGTGAGCGTAAGTTCTCCTAA

SEQ ID NO 2: Mimivirus P4H amino acid sequence in E. coli:

MVLSKSCVSHFRNVGSLNSRDVNLKDDFSYANIDDPYNKPFVLNNLINPTKCQEIMQFANGKLFDSQVLSGTDKNIRN
SQQMVdISKNNPMVKPIFENICRQFNVPFDNAEDLQWRYLPNQYYNEHHDSCCDSSKQCSEFIERGGQRILTVLIYLN
NEFSDGHTYFPNLNQKFKPKTGDALVFYPLANNSNKCHPYSLHAGMPVTSGEKWIANLWFRERKFS

SEQ ID NO 3: Mimi virus Protein sequence in Pichia.

MRFPSIFTAVLFAASSALAAPVNTTTEDETAQIPAEAVIGYSDLEGDFDVAVLPFSNSTNNGLLFINTTIASIAAKEE
GVSLEKREAEAVLSKSCVSHFRNVGSLNSRDVNLKDDFSYANIDDPYNKPFVLNNLINPTKCQEIMQFANGKLFDSQV
LSGTDKNIRNSQQMVdISKNNPMVKPIFENICRQFNVPFDNAEDLQWRYLPNQYYNEHHDSCCDSSKQCSEFIERGGQ
RILTVLIYLNNEFSDGHTYFPNLNQKFKPKTGDALVFYPLANNSNKCHPYSLHAGMPVTSGEKWIANLWFRERKFSHH
HHHH*

SEQ ID NO 4: Codon optimized gene sequence (for Pichia).

ATGGTGCTGTCAAAGTCCTGTGTCAGTCACTTTAGAAATGTTGGATCCTTGAATAGTAGGGATGTCAATCTGAAAGAT
GACTTTTCCTATGCTAATATTGATGATCCCTATAACAAGCCTTTCGTCCTAAATAACCTAATAAACCCTACCAAGTGT
CAAGAGATCATGCAATTTGCCAATGGCAAGTTGTTTGACTCCCAAGTCCTGAGTGGCACGGACAAGAACATACGTAAC
TCTCAACAAATGTGGATATCCAAGAACAACCCTATGGTAAAACCCATTTTCGAGAACATATGCAGGCAGTTTAACGTA
CCCTTTGATAATGCCGAGGACCTACAGGTCGTCCGTTACTTGCCTAATCAATATTATAATGAGCATCATGACTCATGC
TGTGACTCCTCCAAGCAATGCAGTGAATTTATAGAGAGGGGCGGTCAGAGGATTCTGACCGTTTTAATTTACCTAAAC
AACGAGTTCTCAGATGGACACACGTACTTTCCTAATTTAAACCAAAAGTTCAAGCCCAAGACTGGTGATGCTTTGGTT
TTTTACCCTTTAGCCAACAACTCTAATAAATGTCACCCATACAGTCTACACGCAGGTATGCCCGTCACGTCAGGAGAG
AAGTGGATTGCTAATCTGTGGTTTCGTGAGCGTAAGTTCTCCCACCACCACCACCACCACTAA

SEQ ID NO 5: Codon optimized Mimivirus P4H gene sequence with secretion signal (for Pichia).

ATGAGATTCCCATCTATTTTCACCGCTGTCTTGTTCGCTGCCTCCTCTGCATTGGCTGCCCCTGTTAACACTACCACT
GAAGACGAGACTGCTCAAATTCCAGCTGAAGCAGTTATCGGTTACTCTGACCTTGAGGGTGATTTCGACGTCGCTGTT
TTGCCTTTCTCTAACTCCACTAACAACGGTTTGTTGTTCATTAACACCACTATCGCTTCCATTGCTGCTAAGGAAGAG
GGTGTCTCTCTCGAGAAAAGAGAGGCCGAAGCTGTGCTGTCAAAGTCCTGTGTCAGTCACTTTAGAAATGTTGGATCC
TTGAATAGTAGGGATGTCAATCTGAAAGATGACTTTTCCTATGCTAATATTGATGATCCCTATAACAAGCCTTTCGTC
CTAAATAACCTAATAAACCCTACCAAGTGTCAAGAGATCATGCAATTTGCCAATGGCAAGTTGTTTGACTCCCAAGTC
CTGAGTGGCACGGACAAGAACATACGTAACTCTCAACAAATGTGGATATCCAAGAACAACCCTATGGTAAAACCCATT
TTCGAGAACATATGCAGGCAGTTTAACGTACCCTTTGATAATGCCGAGGACCTACAGGTCGTCCGTTACTTGCCTAAT
CAATATTATAATGAGCATCATGACTCATGCTGTGACTCCTCCAAGCAATGCAGTGAATTTATAGAGAGGGGCGGTCAG
AGGATTCTGACCGTTTTAATTTACCTAAACAACGAGTTCTCAGATGGACACACGTACTTTCCTAATTTAAACCAAAAG
TTCAAGCCCAAGACTGGTGATGCTTTGGTTTTTTACCCTTTAGCCAACAACTCTAATAAATGTCACCCATACAGTCTA
CACGCAGGTATGCCCGTCACGTCAGGAGAGAAGTGGATTGCTAATCTGTGGTTTCGTGAGCGTAAGTTCTCCCACCAC
CAC CAC CAC CACTAATAA

SEQ ID NO 6: PBCV-1 protein sequence.

MTNKFISYNKMETREYLLTILFVIACFMVLNLERREGFETSDRPGVCDGKYYEKIDGFLS
DIECDVLINAAIKKGLIKSEVGGATENDPIKLDPKSRNSEQTWFMPGEHEVIDKIQKKTR
EFLNSKKHCIDKYNFEDVQVARYKPGQYYYHHYDGDDCDDACPKDQRLATLMVYLKAPEEGGGGETDFPTLKTKIKPK
KGTSIFFWVADPVTRKLYKETLHAGLPVKSGEKIIANQWIRAVKHHHHHH*

SEQ ID NO 7: Cr-1 protein sequence.

MLLLGLVLALAGHVAAAPSSAMMGTGHTVGFGELKEEWRGEVVHLSWSPRAFLLKNFLSDEECDYIVEKARPKMVKSS
VVDNESGKSVDSEIRTSTGTWFAKGEDSVISKIEKRVAQVTMIPLENHEGLQVLHYHDGQKYEPHYDYFHDPVNAGPE
HGGQRWTMLMYLTTVEEGGETVLPNAEQKVTGDGWSECAKRGLAVKPIKGDALMFYSLKPDGSNDPASLHGSCPTLK
GDKWSATKWIHVAPIGGRHHHHHHH*

SEQ ID NO 8: Arabidopsis thaliana protein sequence.

MARRGLLISFFAIFSVLLQSSTSLISSSSVFVNPSKVKQVSSKPRAFVYEGFLTELECDH
MVSLAKASLKRSAVADNDSGESKFSEVRTSSGTFISKGKDPIVSGIEDKISTWTFLPKEN
GEDIQVLRYEHGQKYDAHFDYFHDKVNIVRGGHRMATILMYLSNVTKGGETVFPDAEIPSRRVLSENKEDLSDCAKRG
IAVKPRKGDALLFFNLHPDAIPDPLSLHGGCPVIEGEKWSATKWIHVDSFDRIVTPSGNCTDMNESCERWAVLGECTK
NPEYMVGTTELPGYCRRSCKACHHHHHH*

SEQ ID NO 9: MMV-398

1 GGATCCTTCA GTAATGTCTT GTTTCTTTTG TTGCAGTGGT GAGCCATTTT GACTTCGTGA
61 AAGTTTCTTT AGAATAGTTG TTTCCAGAGG CCAAACATTC CACCCGTAGT AAAGTGCAAG
121 CGTAGGAAGA CCAAGACTGG CATAAATCAG GTATAAGTGT CGAGCACTGG CAGGTGATCT
181 TCTGAAAGTT TCTACTAGCA GATAAGATCC AGTAGTCATG CATATGGCAA CAATGTACCG
241 TGTGGATCTA AGAACGCGTC CTACTAACCT TCGCATTCGT TGGTCCAGTT TGTTGTTATC
301 GATCAACGTG ACAAGGTTGT CGATTCCGCG TAAGCATGCA TACCCAAGGA CGCCTGTTGC
361 AATTCCAAGT GAGCCAGTTC CAACAATCTT TGTAATATTA GAGCACTTCA TTGTGTTGCG
421 CTTGAAAGTA AAATGCGAAC AAATTAAGAG ATAATCTCGA AACCGCGACT TCAAACGCCA
481 ATATGATGTG CGGCACACAA TAAGCGTTCA TATCCGCTGG GTGACTTTCT CGCTTTAAAA
541 AATTATCCGA AAAAATTTTC TAGAGTGTTG TTACTTTATA CTTCCGGCTC GTATAATACG
601 ACAAGGTGTA AGGAGGACTA AACCATGGCT AAACTCACCT CTGCTGTTCC AGTCCTGACT
661 GCTCGTGATG TTGCTGGTGC TGTTGAGTTC TGGACTGATA GGCTCGGTTT CTCCCGTGAC
721 TTCGTAGAGG ACGACTTTGC CGGTGTTGTA CGTGACGACG TTACCCTGTT CATCTCCGCA
781 GTTCAGGACC AGGTTGTGCC AGACAACACT CTGGCATGGG TATGGGTTCG TGGTCTGGAC
841 GAACTGTACG CTGAGTGGTC TGAGGTCGTG TCTACCAACT TCCGTGATGC ATCTGGTCCA
901 GCTATGACCG AGATCGGTGA ACAGCCCTGG GGTCGTGAGT TTGCACTGCG TGATCCAGCT
961 GGTAACTGCG TGCATTTCGT CGCAGAAGAG CAGGACTAAC AATTGACACC TTACGATTAT
1021 TTAGAGAGTA TTTATTAGTT TTATTGTATG TATACGGATG TTTTATTATC TATTTATGCC
1081 CTTATATTCT GTAACTATCC AAAAGTCCTA TCTTATCAAG CCAGCAATCT ATGTCCGCGA
1141 ACGTCAACTA AAAATAAGCT TTTTATGCTC TTCTCTCTTT TTTTCCCTTC GGTATAATTA
1201 TACCTTGCAT CCACAGATTC TCCTGCCAAA TTTTGCATAA TCCTTTACAA CATGGCTATA
1261 TGGGAGCACT TAGCGCCCTC CAAAACCCAT ATTGCCTACG CATGTATAGG TGTTTTTTCC
1321 ACAATATTTT CTCTGTGCTC TCTTTTTATT AAAGAGAAGC TCTATATCGG AGAAGCTTCT
1381 GTGGCCGTTA TATTCGGCCT TATCGTGGGA CCACATTGCC TGAATTGGTT TGCCCCGGAA
1441 GATTGGGGAA ACTTGGATCT GATTACCTTA GCTGCAGAAA AGGGTACCAC TGAGCGTCAG
1501 ACCCCGTAGA AAAGATCAAA GGATCTTCTT GAGATCCTTT TTTTCTGCGC GTAATCTGCT
1561 GCTTGCAAAC AAAAAAACCA CCGCTACCAG CGGTGGTTTG TTTGCCGGAT CAAGAGCTAC
1621 CAACTCTTTT TCCGAAGGTA ACTGGCTTCA GCAGAGCGCA GATACCAAAT ACTGTTCTTC
1681 TAGTGTAGCC GTAGTTAGGC CACCACTTCA AGAACTCTGT AGCACCGCCT ACATACCTCG
1741 CTCTGCTAAT CCTGTTACCA GTGGCTGCTG CCAGTGGCGA TAAGTCGTGT CTTACCGGGT
1801 TGGACCCAAG ACGATAGTTA CCGGATAAGG CGCAGCGGTC GGGCTGAACG GGGGGTTCGT
1861 GCACACAGCC CAGCTTGGAG CGAACGACCT ACACCGAACT GAGATACCTA CAGCGTGAGC
1921 TATGAGAAAG CGCCACGCTT CCCGAAGGGA GAAAGGCGGA CAGGTATCCG GTAAGCGGCA
1981 GGGTCGGAAC AGGAGAGCGC ACGAGGGAGC TTCCAGGGGG AAACGCCTGG TATCTTTATA
2041 GTCCTGTCGG GTTTCGCCAC CTCTGACTTG AGCGTCGATT TTTGTGATGC TCGTCAGGGG
2101 GGCGGAGCCT ATGGAAAAAC GCCAGCAACG CGGCCTTTTT ACGGTTCCTG GCCTTTTGCT
2161 GGCCTTTTGC TCACATGTTA TTCAGAAGCG ATAGAGAGAC TGCGCTAAGC ATTAATGAGA
2221 TTATTTTTGA GCATTCGTCA ATCAATACCA AACAAGACAA ACGGTATGCC GACTTTTGGA
2281 AGTTTCTTTT TGACCAACTG GCCGTTAGCA TTTCAACGAA CCAAACTTAG TTCATCTTGG
2341 ATGAGATCAC GCTTTTGTCA TATTAGGTTC CAAGACAGCG TTTAAACTGT CAGTTTTGGG
2401 CCATTTGGGG AACATGAAAC TATTTGACCC CACACTCAGA AAGCCCTCAT CTGGAGTGAT
2461 GTTCGGGTGT AATGCGGAGC TTGTTGCATT CGGAAATAAA CAAACATGAA CCTCGCCAGG
2521 GGGGCCAGGA TAGACAGGCT AATAAAGTCA TGGTGTTAGT AGCCTAATAG AAGGAATTGG
2581 AATAAATAAT GTATCTAAAC GCAAACTCCG AGCTGGAAAA ATGTTACCGG CGATGCGCGG
2641 ACAATTTAGA GGCGGCGATC AAGAAACACC TGCTGGGCGA GCAGTCTGGA GCACAGTCTT
2701 CGATGGGCCC GAGATCCCAC CGCGTTCCTG GGTACCGGGA CGTGAGGCAG CGCGACATCC
2761 ATCAAATATA CCAGGCGCCA ACCGAGTCTC TCGGAAAACA GCTTCTGGAT ATCTTCCGCT
2821 GGCGGCGCAA CGACGAATAA TAGTCCCTGG AGGTGACGGA ATATATATGT GTGGAGGGTA
2881 AATCTGACAG GGTGTAGCAA AGGTAATATT TTCCTAAAAC ATGCAATCGG CTGCCCCGCA
2941 ACGGGAAAAA GAATGACTTT GGCACTCTTC ACCAGAGTGG GGTGTCCCGC TCGTGTGTGC
3001 AAATAGGCTC CCACTGGTCA CCCCGGATTT TGCAGAAAAA CAGCAAGTTC CGGGGTGTCT
3061 CACTGGTGTC CGCCAATAAG AGGAGCCGGC AGGCACGGAG TCTACATCAA GCTGTCTCCG
3121 ATACACTCGA CTACCATCCG GGTCTCTCAG AGAGGGGAAT GGCACTATAA ATACCGCCTC
3181 CTTGCGCTCT CTGCCTTCAT CAATCAAATC ATGTTCTCTC CAATTTTGTC CTTGGAAATT
3241 ATTTTAGCTT TGGCTACTTT GCAATCTGTC TTCGCTCAAC AGGAAGCAGT AGATGGTGGT
3301 TGCTCACATT TAGGTCAATC TTACGCAGAT AGAGATGTAT GGAAACCTGA ACCATGTCAA
3361 ATTTGCGTGT GTGACTCAGG TTCAGTGCTC TGCGACGATA TCATATGTGA CGACCAGGAA
3421 TTGGACTGTC CAAACCCAGA GATACCATTC GGTGAATGTT GTGCTGTTTG TCCACAGCCA
3481 CCAACTGCTC CTACAAGACC TCCAAACGGT CAAGGTCCAC AAGGTCCTAA AGGTGATCCG
3541 GGTCCACCTG GTATTCCTGG TAGAAATGGT GACCCTGGAC CTCCCGGTTC CCCAGGTAGC
3601 CCAGGATCAC CTGGGCCTCC TGGAATATGT GAATCCTGCC CAACTGGTGG TCAGAACTAT
3661 AGCCCACAAT ACGAGGCCTA CGACGTCAAA TCTGGTGTTG CTGGAGGAGG TATTGCAGGC
3721 TACCCTGGTC CCGCAGGGCC CCCAGGTCCG CCGGGTCCGC CCGGAACATC AGGTCATCCC
3781 GGAGCCCCTG GTGCACCAGG TTATCAGGGA CCGCCCGGAG AGCCTGGACA AGCTGGTCCC
3841 GCTGGACCCC CTGGTCCACC AGGTGCTATT GGACCAAGTG GTCCTGCCGG AAAAGACGGT
3901 GAATCCGGTA GACCTGGTAG ACCCGGCGAA AGGGGTTTCC CAGGTCCTCC CGGAATGAAG
3961 GGTCCAGCCG GTATGCCCGG TTTTCCTGGG ATGAAGGGTC ACAGAGGATT TGATGGTAGA
4021 AACGGAGAGA AAGGCGAAAC CGGTGCTCCC GGACTGAAGG GTGAAAACGG TGTCCCTGGT
4081 GAGAACGGCG CTCCTGGACC TATGGGTCCA CGTGGTGCTC CAGGAGAAAG AGGCAGACCA
4141 GGATTGCCTG GTGCAGCTGG TGCTAGAGGT AACGATGGTG CCCGTGGTTC CGATGGACAA
4201 CCCGGGCCAC CCGGCCCTCC AGGTACCGCT GGATTTCCTG GAAGCCCTGG TGCTAAGGGG
4261 GAGGTTGGTC CGGCTGGTAG TCCCGGAAGT AGCGGTGCCC CAGGTCAAAG AGGCGAACCA
4321 GGCCCTCAGG GTCACGCAGG AGCACCTGGA CCGCCTGGTC CTCCTGGTTC GAATGGTTCG
4381 CCTGGAGGAA AAGGTGAAAT GGGGCCCGCA GGAATCCCCG GTGCGCCTGG TCTTATTGGT
4441 GCCAGGGGTC CTCCAGGCCC GCCAGGTACA AATGGTGTAC CCGGACAGCG AGGAGCAGCT
4501 GGTGAACCTG GTAAAAACGG TGCCAAAGGA GATCCAGGTC CTCGTGGAGA GCGTGGTGAA
4561 GCTGGCTCTC CCGGTATCGC CGGTCCAAAA GGTGAGGACG GTAAGGACGG TTCCCCTGGT
4621 GAGCCAGGTG CGAACGGACT GCCAGGTGCA GCCGGAGAGC GAGGAGTCCC AGGATTCAGG
4681 GGACCAGCCG GTGCTAACGG CTTGCCTGGT GAAAAAGGGC CCCCTGGTGA TAGGGGAGGA
4741 CCCGGTCCAG CAGGCCCTCG TGGAGTTGCT GGTGAGCCTG GACGTGACGG TTTACCAGGA
4801 GGGCCAGGTT TGAGGGGTAT TCCCGGGTCC CCTGGCGGTC CTGGATCGGA TGGAAAACCA
4861 GGGCCACCAG GTTCGCAGGG TGAAACAGGA CGTCCAGGCC CACCCGGCTC ACCTGGTCCA
4921 AGGGGTCAGC CTGGTGTCAT GGGTTTCCCC GGTCCAAAGG GTAATGACGG AGCACCGGGT
4981 AAAAATGGTG AACGTGGTGG CCCAGGTGGT CCAGGACCCC AAGGTCCAGC TGGAAAAAAC
5041 GGTGAGACAG GTCCTCAAGG ACCTCCAGGA CCTACCGGTC CTAGCGGAGA TAAGGGAGAT
5101 ACGGGACCGC CAGGACCTCA AGGATTGCAA GGTTTGCCTG GTACATCTGG CCCTCCCGGA
5161 GAAAATGGTA AGCCTGGAGA GCCAGGACCA AAAGGCGAAG CTGGAGCCCC AGGTATCCCC
5221 GGAGGTAAGG GAGACTCAGG TGCTCCGGGT GAGCGTGGTC CTCCGGGTGC CGGTGGTCCA
5281 CCTGGACCTA GAGGTGGTGC CGGGCCGCCA GGTCCTGAAG GTGGTAAAGG TGCTGCTGGT
5341 CCACCGGGAC CGCCTGGCTC TGCTGGTACT CCTGGCTTGC AGGGAATGCC AGGAGAGAGA
5401 GGTGGACCTG GAGGTCCCGG TCCGAAGGGT GATAAAGGGG AGCCAGGATC ATCCGGTGTT
5461 GACGGCGCAC CTGGTAAAGA CGGACCAAGG GGACCAACGG GTCCAATCGG ACCACCAGGA
5521 CCCGCTGGCC AGCCAGGAGA TAAAGGCGAG TCCGGAGCAC CCGGTGTTCC TGGTATAGCT
5581 GGACCCAGGG GTGGTCCCGG TGAAAGAGGT GAACAGGGCC CACCGGGTCC CGCCGGTTTC
5641 CCTGGCGCCC CTGGTCAAAA TGGAGAACCA GGTGCAAAGG GCGAGAGAGG AGCCCCAGGA
5701 GAAAAGGGTG AGGGAGGACC ACCCGGTGCT GCCGGTCCAG CTGGGGGTTC AGGTCCTGCT
5761 GGACCACCAG GTCCACAGGG CGTTAAAGGT GAGAGAGGAA GTCCAGGTGG TCCTGGAGCT
5821 GCTGGATTCC CAGGTGGCCG TGGACCTCCT GGTCCCCCTG GATCGAATGG TAATCCTGGT
5881 CCGCCAGGTA GTTCGGGTGC TCCTGGGAAG GACGGTCCAC CTGGCCCCCC AGGTAGTAAC
5941 GGTGCACCTG GTAGTCCAGG TATATCCGGA CCTAAAGGAG ATTCCGGTCC ACCAGGCGAA
6001 AGAGGGGCCC CAGGCCCACA GGGTCCACCA GGAGCCCCCG GTCCTCTGGG TATTGCTGGT
6061 CTTACTGGTG CACGTGGACT GGCCGGTCCA CCCGGAATGC CTGGAGCAAG AGGTTCACCT
6121 GGACCACAAG GTATTAAAGG AGAGAACGGT AAACCTGGAC CTTCCGGTCA AAACGGAGAG
6181 CGGGGACCCC CAGGCCCCCA AGGTCTGCCA GGACTAGCTG GTACCGCAGG GGAACCAGGA
6241 AGAGATGGAA ATCCAGGTTC AGACGGACTA CCCGGTAGAG ATGGTGCACC GGGGGCCAAG
6301 GGCGACAGGG GTGAGAATGG ATCTCCTGGT GCGCCAGGGG CACCAGGCCA CCCAGGTCCC
6361 CCAGGTCCTG TGGGCCCTGC TGGAAAGTCA GGTGACAGGG GAGAGACAGG CCCGGCTGGT
6421 CCATCTGGCG CACCCGGACC AGCTGGTTCC AGAGGCCCAC CTGGTCCGCA AGGCCCTAGA
6481 GGTGACAAGG GAGAGACTGG AGAACGAGGT GCTATGGGTA TCAAGGGTCA TAGAGGTTTT
6541 CCGGGTAATC CCGGCGCCCC AGGTTCTCCT GGTCCAGCTG GCCATCAAGG TGCAGTCGGA
6601 TCGCCCGGCC CAGCCGGTCC CAGGGGCCCT GTTGGTCCAT CCGGTCCTCC AGGAAAGGAT
6661 GGTGCTTCTG GACACCCAGG ACCTATCGGA CCTCCGGGTC CTAGAGGTAA TAGAGGAGAA
6721 CGTGGATCCG AGGGTAGTCC TGGTCACCCT GGTCAACCTG GCCCACCAGG GCCTCCAGGT
6781 GCACCCGGTC CATGTTGTGG TGCAGGCGGT GTGGCTGCAA TTGCTGGTGT GGGTGCTGAA
6841 AAGGCCGGCG GTTTCGCTCC ATATTATGGT GATGGTTACA TTCCTGAAGC TCCTAGAGAC
6901 GGACAAGCAT ACGTTAGAAA GGACGGTGAG TGGGTGTTGC TGTCCACCTT CTTATAATCA
6961 AGAGGATGTC AGAATGCCAT TTGCCTGAGA GATGCAGGCT TCATTTTTGA TACTTTTTTA
7021 TTTGTAACCT ATATAGTATA GGATTTTTTT TGTCATTTTG TTTCTTCTCG TACGAGCTTG
7081 CTCCTGATCA GCCTATCTCG CAGCTGATGA ATATCTTGTG GTAGGGGTTT GGGAAAATCA
7141 TTCGAGTTTG ATGTTTTTCT TGGTATTTCC CACTCCTCTT CAGAGTACAG AAGATTAAGT
7201 GAGACGTTCG TTTGTGCTCC GGA

SEQ ID NO 10: MMV-589

1 GGATCCTTCA GTAATGTCTT GTTTCTTTTG TTGCAGTGGT GAGCCATTTT GACTTCGTGA
61 AAGTTTCTTT AGAATAGTTG TTTCCAGAGG CCAAACATTC CACCCGTAGT AAAGTGCAAG
121 CGTAGGAAGA CCAAGACTGG CATAAATCAG GTATAAGTGT CGAGCACTGG CAGGTGATCT
181 TCTGAAAGTT TCTACTAGCA GATAAGATCC AGTAGTCATG CATATGGCAA CAATGTACCG
241 TGTGGATCTA AGAACGCGTC CTACTAACCT TCGCATTCGT TGGTCCAGTT TGTTGTTATC
301 GATCAACGTG ACAAGGTTGT CGATTCCGCG TAAGCATGCA TACCCAAGGA CGCCTGTTGC
361 AATTCCAAGT GAGCCAGTTC CAACAATCTT TGTAATATTA GAGCACTTCA TTGTGTTGCG
421 CTTGAAAGTA AAATGCGAAC AAATTAAGAG ATAATCTCGA AACCGCGACT TCAAACGCCA
481 ATATGATGTG CGGCACACAA TAAGCGTTCA TATCCGCTGG GTGACTTTCT CGCTTTAAAA
541 AATTATCCGA AAAAATTTTC TAGAGTGTTG TTACTTTATA CTTCCGGCTC GTATAATACG
601 ACAAGGTGTA AGGAGGACTA AACCATGGCT AAACTCACCT CTGCTGTTCC AGTCCTGACT
661 GCTCGTGATG TTGCTGGTGC TGTTGAGTTC TGGACTGATA GGCTCGGTTT CTCCCGTGAC
721 TTCGTAGAGG ACGACTTTGC CGGTGTTGTA CGTGACGACG TTACCCTGTT CATCTCCGCA
781 GTTCAGGACC AGGTTGTGCC AGACAACACT CTGGCATGGG TATGGGTTCG TGGTCTGGAC
841 GAACTGTACG CTGAGTGGTC TGAGGTCGTG TCTACCAACT TCCGTGATGC ATCTGGTCCA
901 GCTATGACCG AGATCGGTGA ACAGCCCTGG GGTCGTGAGT TTGCACTGCG TGATCCAGCT
961 GGTAACTGCG TGCATTTCGT CGCAGAAGAG CAGGACTAAC AATTGACACC TTACGATTAT
1021 TTAGAGAGTA TTTATTAGTT TTATTGTATG TATACGGATG TTTTATTATC TATTTATGCC
1081 CTTATATTCT GTAACTATCC AAAAGTCCTA TCTTATCAAG CCAGCAATCT ATGTCCGCGA
1141 ACGTCAACTA AAAATAAGCT TTTTATGCTC TTCTCTCTTT TTTTCCCTTC GGTATAATTA
1201 TACCTTGCAT CCACAGATTC TCCTGCCAAA TTTTGCATAA TCCTTTACAA CATGGCTATA
1261 TGGGAGCACT TAGCGCCCTC CAAAACCCAT ATTGCCTACG CATGTATAGG TGTTTTTTCC
1321 ACAATATTTT CTCTGTGCTC TCTTTTTATT AAAGAGAAGC TCTATATCGG AGAAGCTTCT
1381 GTGGCCGTTA TATTCGGCCT TATCGTGGGA CCACATTGCC TGAATTGGTT TGCCCCGGAA
1441 GATTGGGGAA ACTTGGATCT GATTACCTTA GCTGCAGAAA AGGGTACCAC TGAGCGTCAG
1501 ACCCCGTAGA AAAGATCAAA GGATCTTCTT GAGATCCTTT TTTTCTGCGC GTAATCTGCT
1561 GCTTGCAAAC AAAAAAACCA CCGCTACCAG CGGTGGTTTG TTTGCCGGAT CAAGAGCTAC
1621 CAACTCTTTT TCCGAAGGTA ACTGGCTTCA GCAGAGCGCA GATACCAAAT ACTGTTCTTC
1681 TAGTGTAGCC GTAGTTAGGC CACCACTTCA AGAACTCTGT AGCACCGCCT ACATACCTCG
1741 CTCTGCTAAT CCTGTTACCA GTGGCTGCTG CCAGTGGCGA TAAGTCGTGT CTTACCGGGT
1801 TGGACCCAAG ACGATAGTTA CCGGATAAGG CGCAGCGGTC GGGCTGAACG GGGGGTTCGT
1861 GCACACAGCC CAGCTTGGAG CGAACGACCT ACACCGAACT GAGATACCTA CAGCGTGAGC
1921 TATGAGAAAG CGCCACGCTT CCCGAAGGGA GAAAGGCGGA CAGGTATCCG GTAAGCGGCA
1981 GGGTCGGAAC AGGAGAGCGC ACGAGGGAGC TTCCAGGGGG AAACGCCTGG TATCTTTATA
2041 GTCCTGTCGG GTTTCGCCAC CTCTGACTTG AGCGTCGATT TTTGTGATGC TCGTCAGGGG
2101 GGCGGAGCCT ATGGAAAAAC GCCAGCAACG CGGCCTTTTT ACGGTTCCTG GCCTTTTGCT
2161 GGCCTTTTGC TCACATGTTA TTCAGAAGCG ATAGAGAGAC TGCGCTAAGC ATTAATGAGA
2221 TTATTTTTGA GCATTCGTCA ATCAATACCA AACAAGACAA ACGGTATGCC GACTTTTGGA
2281 AGTTTCTTTT TGACCAACTG GCCGTTAGCA TTTCAACGAA CCAAACTTAG TTCATCTTGG
2341 ATGAGATCAC GCTTTTGTCA TATTAGGTTC CAAGACAGCG TTTAAACTGT CAGTTTTGGG
2401 CCATTTGGGG AACATGAAAC TATTTGACCC CACACTCAGA AAGCCCTCAT CTGGAGTGAT
2461 GTTCGGGTGT AATGCGGAGC TTGTTGCATT CGGAAATAAA CAAACATGAA CCTCGCCAGG
2521 GGGGCCAGGA TAGACAGGCT AATAAAGTCA TGGTGTTAGT AGCCTAATAG AAGGAATTGG
2581 AATAAATAAT GTATCTAAAC GCAAACTCCG AGCTGGAAAA ATGTTACCGG CGATGCGCGG
2641 ACAATTTAGA GGCGGCGATC AAGAAACACC TGCTGGGCGA GCAGTCTGGA GCACAGTCTT
2701 CGATGGGCCC GAGATCCCAC CGCGTTCCTG GGTACCGGGA CGTGAGGCAG CGCGACATCC
2761 ATCAAATATA CCAGGCGCCA ACCGAGTCTC TCGGAAAACA GCTTCTGGAT ATCTTCCGCT
2821 GGCGGCGCAA CGACGAATAA TAGTCCCTGG AGGTGACGGA ATATATATGT GTGGAGGGTA
2881 AATCTGACAG GGTGTAGCAA AGGTAATATT TTCCTAAAAC ATGCAATCGG CTGCCCCGCA
2941 ACGGGAAAAA GAATGACTTT GGCACTCTTC ACCAGAGTGG GGTGTCCCGC TCGTGTGTGC
3001 AAATAGGCTC CCACTGGTCA CCCCGGATTT TGCAGAAAAA CAGCAAGTTC CGGGGTGTCT
3061 CACTGGTGTC CGCCAATAAG AGGAGCCGGC AGGCACGGAG TCTACATCAA GCTGTCTCCG
3121 ATACACTCGA CTACCATCCG GGTCTCTCAG AGAGGGGAAT GGCACTATAA ATACCGCCTC
3181 CTTGCGCTCT CTGCCTTCAT CAATCAAATC ATGTTCTCTC CAATTTTGTC CTTGGAAATT
3241 ATTTTAGCTT TGGCTACTTT GCAATCTGTC TTCGCTCAAC AGGAAGCAGT AGATGGTGGT
3301 TGCTCACATT TAGGTCAATC TTACGCAGAT AGAGATGTAT GGAAACCTGA ACCATGTCAA
3361 ATTTGCGTGT GTGACTCAGG TTCAGTGCTC TGCGACGATA TCATATGTGA CGACCAGGAA
3421 TTGGACTGTC CAAACCCAGA GATACCATTC GGTGAATGTT GTGCTGTTTG TCCACAGCCA
3481 CCAACTGCTC CTACAAGACC TCCAAACGGT CAAGGTCCAC AAGGTCCTAA AGGTGATCCG
3541 GGTCCACCTG GTATTCCTGG TAGAAATGGT GACCCTGGAC CTCCCGGTTC CCCAGGTAGC
3601 CCAGGATCAC CTGGGCCTCC TGGAATATGT GAATCCTGCC CAACTGGTGG TCAGAACTAT
3661 AGCCCACAAT ACGAGGCCTA CGACGTCAAA TCTGGTGTTG CTGGAGGAGG TATTGCAGGC
3721 TACCCTGGTC CCGCAGGGCC CCCAGGTCCG CCGGGTCCGC CCGGAACATC AGGTCATCCC
3781 GGAGCCCCTG GTGCACCAGG TTATCAGGGA CCGCCCGGAG AGCCTGGACA AGCTGGTCCC
3841 GCTGGACCCC CTGGTCCACC AGGTGCTATT GGACCAAGTG GTCCTGCCGG AAAAGACGGT
3901 GAATCCGGTA GACCTGGTAG ACCCGGCGAA AGGGGTTTCC CAGGTCCTCC CGGAATGAAG
3961 GGTCCAGCCG GTATGCCCGG TTTTCCTGGG ATGAAGGGTC ACAGAGGATT TGATGGTAGA
4021 AACGGAGAGA AAGGCGAAAC CGGTGCTCCC GGACTGAAGG GTGAAAACGG TGTCCCTGGT
4081 GAGAACGGCG CTCCTGGACC TATGGGTCCA CGTGGTGCTC CAGGAGAAAG AGGCAGACCA
4141 GGATTGCCTG GTGCAGCTGG TGCTAGAGGT AACGATGGTG CCCGTGGTTC CGATGGACAA
4201 CCCGGGCCAC CCGGCCCTCC AGGTACCGCT GGATTTCCTG GAAGCCCTGG TGCTAAGGGG
4261 GAGGTTGGTC CGGCTGGTAG TCCCGGAAGT AGCGGTGCCC CAGGTCAAAG AGGCGAACCA
4321 GGCCCTCAGG GTCACGCAGG AGCACCTGGA CCGCCTGGTC CTCCTGGTTC GAATGGTTCG
4381 CCTGGAGGAA AAGGTGAAAT GGGGCCCGCA GGAATCCCCG GTGCGCCTGG TCTTATTGGT
4441 GCCAGGGGTC CTCCAGGCCC GCCAGGTACA AATGGTGTAC CCGGACAGCG AGGAGCAGCT
4501 GGTGAACCTG GTAAAAACGG TGCCAAAGGA GATCCAGGTC CTCGTGGAGA GCGTGGTGAA
4561 GCTGGCTCTC CCGGTATCGC CGGTCCAAAA GGTGAGGACG GTAAGGACGG TTCCCCTGGT
4621 GAGCCAGGTG CGAACGGACT GCCAGGTGCA GCCGGAGAGC GAGGAGTCCC AGGATTCAGG
4681 GGACCAGCCG GTGCTAACGG CTTGCCTGGT GAAAAAGGGC CCCCTGGTGA TAGGGGAGGA
4741 CCCGGTCCAG CAGGCCCTCG TGGAGTTGCT GGTGAGCCTG GACGTGACGG TTTACCAGGA
4801 GGGCCAGGTT TGAGGGGTAT TCCCGGGTCC CCTGGCGGTC CTGGATCGGA TGGAAAACCA
4861 GGGCCACCAG GTTCGCAGGG TGAAACAGGA CGTCCAGGCC CACCCGGCTC ACCTGGTCCA
4921 AGGGGTCAGC CTGGTGTCAT GGGTTTCCCC GGTCCAAAGG GTAATGACGG AGCACCGGGT
4981 AAAAATGGTG AACGTGGTGG CCCAGGTGGT CCAGGACCCC AAGGTCCAGC TGGAAAAAAC
5041 GGTGAGACAG GTCCTCAAGG ACCTCCAGGA CCTACCGGTC CTAGCGGAGA TAAGGGAGAT
5101 ACGGGACCGC CAGGACCTCA AGGATTGCAA GGTTTGCCTG GTACATCTGG CCCTCCCGGA
5161 GAAAATGGTA AGCCTGGAGA GCCAGGACCA AAAGGCGAAG CTGGAGCCCC AGGTATCCCC
5221 GGAGGTAAGG GAGACTCAGG TGCTCCGGGT GAGCGTGGTC CTCCGGGTGC CGGTGGTCCA
5281 CCTGGACCTA GAGGTGGTGC CGGGCCGCCA GGTCCTGAAG GTGGTAAAGG TGCTGCTGGT
5341 CCACCGGGAC CGCCTGGCTC TGCTGGTACT CCTGGCTTGC AGGGAATGCC AGGAGAGAGA
5401 GGTGGACCTG GAGGTCCCGG TCCGAAGGGT GATAAAGGGG AGCCAGGATC ATCCGGTGTT
5461 GACGGCGCAC CTGGTAAAGA CGGACCAAGG GGACCAACGG GTCCAATCGG ACCACCAGGA
5521 CCCGCTGGCC AGCCAGGAGA TAAAGGCGAG TCCGGAGCAC CCGGTGTTCC TGGTATAGCT
5581 GGACCCAGGG GTGGTCCCGG TGAAAGAGGT GAACAGGGCC CACCGGGTCC CGCCGGTTTC
5641 CCTGGCGCCC CTGGTCAAAA TGGAGAACCA GGTGCAAAGG GCGAGAGAGG AGCCCCAGGA
5701 GAAAAGGGTG AGGGAGGACC ACCCGGTGCT GCCGGTCCAG CTGGGGGTTC AGGTCCTGCT
5761 GGACCACCAG GTCCACAGGG CGTTAAAGGT GAGAGAGGAA GTCCAGGTGG TCCTGGAGCT
5821 GCTGGATTCC CAGGTGGCCG TGGACCTCCT GGTCCCCCTG GATCGAATGG TAATCCTGGT
5881 CCGCCAGGTA GTTCGGGTGC TCCTGGGAAG GACGGTCCAC CTGGCCCCCC AGGTAGTAAC
5941 GGTGCACCTG GTAGTCCAGG TATATCCGGA CCTAAAGGAG ATTCCGGTCC ACCAGGCGAA
6001 AGAGGGGCCC CAGGCCCACA GGGTCCACCA GGAGCCCCCG GTCCTCTGGG TATTGCTGGT
6061 CTTACTGGTG CACGTGGACT GGCCGGTCCA CCCGGAATGC CTGGAGCAAG AGGTTCACCT
6121 GGACCACAAG GTATTAAAGG AGAGAACGGT AAACCTGGAC CTTCCGGTCA AAACGGAGAG
6181 CGGGGACCCC CAGGCCCCCA AGGTCTGCCA GGACTAGCTG GTACCGCAGG GGAACCAGGA
6241 AGAGATGGAA ATCCAGGTTC AGACGGACTA CCCGGTAGAG ATGGTGCACC GGGGGCCAAG
6301 GGCGACAGGG GTGAGAATGG ATCTCCTGGT GCGCCAGGGG CACCAGGCCA CCCAGGTCCC
6361 CCAGGTCCTG TGGGCCCTGC TGGAAAGTCA GGTGACAGGG GAGAGACAGG CCCGGCTGGT
6421 CCATCTGGCG CACCCGGACC AGCTGGTTCC AGAGGCCCAC CTGGTCCGCA AGGCCCTAGA
6481 GGTGACAAGG GAGAGACTGG AGAACGAGGT GCTATGGGTA TCAAGGGTCA TAGAGGTTTT
6541 CCGGGTAATC CCGGCGCCCC AGGTTCTCCT GGTCCAGCTG GCCATCAAGG TGCAGTCGGA
6601 TCGCCCGGCC CAGCCGGTCC CAGGGGCCCT GTTGGTCCAT CCGGTCCTCC AGGAAAGGAT
6661 GGTGCTTCTG GACACCCAGG ACCTATCGGA CCTCCGGGTC CTAGAGGTAA TAGAGGAGAA
6721 CGTGGATCCG AGGGTAGTCC TGGTCACCCT GGTCAACCTG GCCCACCAGG GCCTCCAGGT
6781 GCACCCGGTC CATGTTGTGG TGCAGGCGGT GTGGCTGCAA TTGCTGGTGT GGGTGCTGAA
6841 AAGGCCGGCG GTTTCGCTCC ATATTATGGT TAATCAAGAG GATGTCAGAA TGCCATTTGC
6901 CTGAGAGATG CAGGCTTCAT TTTTGATACT TTTTTATTTG TAACCTATAT AGTATAGGAT
6961 TTTTTTTGTC ATTTTGTTTC TTCTCGTACG AGCTTGCTCC TGATCAGCCT ATCTCGCAGC
7021 TGATGAATAT CTTGTGGTAG GGGTTTGGGA AAATCATTCG AGTTTGATGT TTTTCTTGGT
7081 ATTTCCCACT CCTCTTCAGA GTACAGAAGA TTAAGTGAGA CGTTCGTTTG TGCTCCGGA

SEQ ID NO 11: MMV-619

1 GTTTTAGCCT TAGACATGAC TGTTCCTCAG TTCAAGTTGG GCACTTACGA GAAGACCGGT
61 CTTGCTAGAT TCTAATCAAG AGGATGTCAG AATGCCATTT GCCTGAGAGA TGCAGGCTTC
121 ATTTTTGATA CTTTTTTATT TGTAACCTAT ATAGTATAGG ATTTTTTTTG TCATTTTGTT
181 TCTTCTCGTA CGAGCTTGCT CCTGATCAGC CTATCTCGCA GCTGATGAAT ATCTTGTGGT
241 AGGGGTTTGG GAAAATCATT CGAGTTTGAT GTTTTTCTTG GTATTTCCCA CTCCTCTTCA
301 GAGTACAGAA GATTAAGTGA GACCTTCGTT TGTGCGGATC CGGAACGGAA CGTATCTTAG
361 CATGGTTGTG CGACAGATTC ACTGTGAAAG ACTGTTCATT ATACCCACGT TTCACTGGGA
421 GATGTAAGCC TTAGGTGTTT TACCCTGATT AGATAATACA ATAACCAACA GAAATACGAG
481 AATCTAAACT AATTTCGATG ATTCATTTTT CTTTTTACCG CGCTGCCTCT TTTGGCAATT
541 CTTTCACCTA TATTCTACCT TCTCTTTCCT TTTGTTCTAA ACTTATTACC AGCTACATAT
601 GACATTTCCC TTGCTACCTG CATACGCAAG TGTTGCAGAG TTTGATAATT CCTTGAGTTT
661 GGTAGGAAAA GCCGTGTTTC CCTATGCTGC TGACCAGCTG CACAACCTGA TCAAGTTCAC
721 TCAATCGACT GAGCTTCAAG TTAATGTGCA AGTTGAGTCA TCCGTTACAG AGGACCAATT
781 TGAGGAGCTG ATCGACAACT TGCTCAAGTT GTACAATAAT GGTATCAATG AAGTGATTTT
841 GGACCTAGAT TTGGCAGAAA GAGTTGTCCA AAGGATCCCA GGCGCTAGGG TTATCTATAG
901 GACCCTGGTT GATAAAGTTG CATCCTTGCC CGCTAATGCT AGTATCGCTG TGCCTTTTTC
961 TTCTCCACTG GGCGATTTGA AAAGTTTCAC TAATGGCGGT AGTAGAACTG TTTATGCTTT
1021 TTCTGAGACC GCAAAGTTGG TAGATGTGAC TTCCACTGTT GCTTCTGGTA TAATCCCCAT
1081 TATTGATGCT CGGCAATTGA CTACTGAATA CGAACTTTCT GAAGATGTCA AAAAGTTCCC
1141 TGTCAGTGAA ATTTTGTTGG CGTCTTTGAC TACTGACCGC CCCGATGGTC TATTCACTAC
1201 TTTGGTGGCT GACTCTTCTA ATTACTCGTT GGGCCTGGTG TACTCGTCCA AAAAGTCTAT
1261 TCCGGAGGCT ATAAGGACAC AAACTGGAGT CTACCAATCT CGTCGTCACG GTTTGTGGTA
1321 TAAAGGTGCT ACATCTGGAG CAACTCAAAA GTTGCTGGGT ATCGAATTGG ATTGTGATGG
1381 AGACTGCTTG AAATTTGTGG TTGAACAAAC AGGTGTTGGT TTCTGTCACT TGGAACGCAC
1441 TTCCTGTTTT GGCCAATCAA AGGGTCTTAG AGCCATGGAA GCCACCTTGT GGGATCGTAA
1501 GAGCAATGCT CCAGAAGGTT CTTATACCAA ACGGTTATTT GACGACGAAG TTTTGTTGAA
1561 CGCTAAAATT AGGGAGGAAG CTGATGAACT TGCAGAAGCT AAATCCAAGG AAGATATAGC
1621 CTGGGAATGT GCTGACTTAT TTTATTTTGC ATTAGTTAGA TGTGCCAAGT ACGGTGTGAC
1681 GTTGGACGAG GTGGAGAGAA ACCTGGATAT GAAGTCCCTA AAGGTCACTA GAAGGAAAGG
1741 AGATGCCAAG CCAGGATACA CCAAGGAACA ACCTAAAGAA GAATCCAAAC CTAAAGAAGT
1801 CCCTTCTGAA GGTCGTATTG AATTGTGCAA AATTGACGTT TCTAAGGCCT CCTCACAAGA
1861 AATTGAAGAT GCCCTTCGTC GTCCTATCCA GAAAACGGAA CAGATTATGG AATTAGTCAA
1921 ACCAATTGTC GACAATGTTC GTCAAAATGG TGACAAAGCC CTTTTAGAAC TAACTGCCAA
1981 GTTTGATGGA GTCGCTTTGA AGACACCTGT GTTAGAAGCT CCTTTCCCAG AGGAACTTAT
2041 GCAATTGCCA GATAACGTTA AGAGAGCCAT TGATCTCTCT ATAGATAACG TCAGGAAATT
2101 CCATGAAGCT CAACTAACGG AGACGTTGCA AGTTGAGACT TGCCCTGGTG TAGTCTGCTC
2161 TCGTTTTGCA AGACCTATTG AGAAAGTTGG CCTCTATATT CCTGGTGGAA CCGCAATTCT
2221 GCCTTCCACT TCCCTGATGC TGGGTGTTCC TGCCAAAGTT GCTGGTTGCA AAGAAATTGT
2281 TTTTGCATCT CCACCTAAGA AGGATGGTAC CCTTACCCCA GAAGTCATCT ACGTTGCCCA
2341 CAAGGTTGGT GCTAAGTGTA TCGTGCTAGC AGGAGGCGCC CAGGCAGTAG CTGCTATGGC
2401 TTACGGAACA GAAACTGTTC CTAAGTGTGA CAAAATATTT GGTCCAGGAA ACCAGTTCGT
2461 TACTGCTGCC AAGATGATGG TTCAAAATGA CACATCAGCC CTGTGTAGTA TTGACATGCC
2521 TGCTGGGCCT TCTGAAGTTC TAGTTATTGC TGATAAATAC GCTGATCCAG ATTTCGTTGC
2581 CTCAGACCTT CTGTCTCAAG CTGAACATGG TATTGATTCC CAGGTGATTC TGTTGGCTGT
2641 CGATATGACA GACAAGGAGC TTGCCAGAAT TGAAGATGCT GTTCACAACC AAGCTGTGCA
2701 GTTGCCAAGG GTTGAAATTG TACGCAAGTG TATTGCACAC TCTACAACCC TATCGGTTGC
2761 AACCTACGAG CAGGCTTTGG AAATGTCCAA TCAGTACGCT CCTGAACACT TGATCCTGCA
2821 AATCGAGAAT GCTTCTTCTT ATGTTGATCA AGTACAACAC GCTGGATCTG TGTTTGTTGG
2881 TGCCTACTCT CCAGAGAGTT GTGGAGATTA CTCCTCCGGT ACCAACCACA CTTTGCCAAC
2941 GTACGGATAT GCCCGTCAAT ACAGCGGAGT TAACACTGCA ACCTTCCAGA AGTTCATCAC
3001 TTCACAAGAC GTAACTCCTG AGGGACTGAA ACATATTGGC CAAGCAGTGA TGGATCTGGC
3061 TGCTGTTGAA GGTCTAGATG CTCACCGCAA TGCTGTTAAG GTTCGTATGG AGAAACTGGG
3121 ACTTATTTAA CTGCAGTATA CTGAGTTTGT TAATGATACA ATAAACTGTT ATAGTACATA
3181 CAATTGAAAC TCTCTTATCT ATACTGGGGG ACCTTCTCGC AGAATGGTAT AAATATCTAC
3241 TAACTGACTG TCGTACGGCC TAGGGGTCTC TTCTTCGATT ATTTGCAGGT CGGAACATCC
3301 TTCGTCTGAT GCGGATCTCC TGAGACAAAG TTCACGGGTA TCTAGTATTC TATCAGCATA
3361 AATGGAGGAC CTTTCTAAAC TAAACTTTGA ATCGTCTCCA GCAGCATCCT CGCATTCGAG
3421 TATCTATGAT TGGAAGTATG GGAATGGTGA TACCCGCATT CTTCAGTGTC TTGAGGTCTC
3481 CTATCAGATT ATGCCCAACT AAAGCAACCG GAGGAGGAGA TTTCATGGTA AATTTCTCTG
3541 ACTTTTGGTC ATCAGTAGAC TCGAACTGTG AGACTATCTC GGTTATGACA GCAGAAATGT
3601 CCTTCTTGGA GACAGTAAAT GAAGTCCCAC CAATAAAGAA ATCCTTGTTA TCAGGAACAA
3661 ACTTCTTGTT TCGAACTTTT TCGGTGCCTT GAACTATAAA ATGTAGAGTG GATATGTCGG
3721 GTAGGAATGG AGCGGGCAAA TGCTTACCTT CTGGACCTTC AAGAGGTATG TAGGGTTTGT
3781 AGATACTGAT GCCAACTTCA GTGACAACGT TGCTATTTCG TTCAAACCAT TCCGAATCCA
3841 GAGAAATCAA AGTTGTTTGT CTACTATTGA TCCAAGCCAG TGCGGTCTTG AAACTGACAA
3901 TAGTGTGCTC GTGTTTTGAG GTCATCTTTG TATGAATAAA TCTAGTCTTT GATCTAAATA
3961 ATCTTGACGA GCCAGACGAT AATACCAATC TAAACTCTTT AAACGTTAAA GGACAAGTAT
4021 GTCTGCCTGT ATTAAACCCC AAATCAGCTC GTAGTCTGAT CCTCATCAAC TTGAGGGGCA
4081 CTATCTTGTT TTAGAGAAAT TTGCGGAGAT GCGATATCGA GAAAAAGGTA CGCTGATTTT
4141 AAACGTGAAA TTTATCTCAA GATCTTCACT GACTCGCTGC GCTCGGTCGT TCGGCTGCGG
4201 CGAGCGGTAT CAGCTCACTC AAAGGCGGTA ATACGGTTAT CCACAGAATC AGGGGATAAC
4261 GCAGGAAAGA ACATGTGAGC AAAAGGCCAG CAAAAGGCCA GGAACCGTAA AAAGGCCGCG
4321 TTGCTGGCGT TTTTCCATAG GCTCCGCCCC CCTGACGAGC ATCACAAAAA TCGACGCTCA
4381 AGTCAGAGGT GGCGAAACCC GACAGGACTA TAAAGATACC AGGCGTTTCC CCCTGGAAGC
4441 TCCCTCGTGC GCTCTCCTGT TCCGACCCTG CCGCTTACCG GATACCTGTC CGCCTTTCTC
4501 CCTTCGGGAA GCGTGGCGCT TTCTCATAGC TCACGCTGTA GGTATCTCAG TTCGGTGTAG
4561 GTCGTTCGCT CCAAGCTGGG CTGTGTGCAC GAACCCCCCG TTCAGCCCGA CCGCTGCGCC
4621 TTATCCGGTA ACTATCGTCT TGAGTCCAAC CCGGTAAGAC ACGACTTATC GCCACTGGCA
4681 GCAGCCACTG GTAACAGGAT TAGCAGAGCG AGGTATGTAG GCGGTGCTAC AGAGTTCTTG
4741 AAGTGGTGGC CTAACTACGG CTACACTAGA AGAACAGTAT TTGGTATCTG CGCTCTGCTG
4801 AAGCCAGTTA CCTTCGGAAA AAGAGTTGGT AGCTCTTGAT CCGGCAAACA AACCACCGCT
4861 GGTAGCGGTG GTTTTTTTGT TTGCAAGCAG CAGATTACGC GCAGAAAAAA AGGATCTCAA
4921 GAAGATCCTT TGATCTTTTC TACGGGGTCT GACGCTCAGT GGAACGAAAA CTCACGTTAA
4981 GGGATTTTGG TCATGAGATT ATCAAAAAGG ATCTTCACCT AGATCCTTTT AAATTAAAAA
5041 TGAAGTTTTA AATCAATCTA AAGTATATAT GAGTAAACTT GGTCTGACAG TTACCAATGC
5101 TTAATCAGTG AGGCACCTAT CTCAGCGATC TGTCTATTTC GTTCATCCAT AGTTGCCTGA
5161 CTCCCCGTCG TGTAGATAAC TACGATACGG GAGGGCTTAC CATCTGGCCC CAGTGCTGCA
5221 ATGATACCGC GAGACCCACG CTCACCGGCT CCAGATTTAT CAGCAATAAA CCAGCCAGCC
5281 GGAAGGGCCG AGCGCAGAAG TGGTCCTGCA ACTTTATCCG CCTCCATCCA GTCTATTAAT
5341 TGTTGCCGGG AAGCTAGAGT AAGTAGTTCG CCAGTTAATA GTTTGCGCAA CGTTGTTGCC
5401 ATTGCTACAG GCATCGTGGT GTCACGCTCG TCGTTTGGTA TGGCTTCATT CAGCTCCGGT
5461 TCCCAACGAT CAAGGCGAGT TACATGATCC CCCATGTTGT GCAAAAAAGC GGTTAGCTCC
5521 TTCGGTCCTC CGATCGTTGT CAGAAGTAAG TTGGCCGCAG TGTTATCACT CATGGTTATG
5581 GCAGCACTGC ATAATTCTCT TACTGTCATG CCATCCGTAA GATGCTTTTC TGTGACTGGT
5641 GAGTACTCAA CCAAGTCATT CTGAGAATAG TGTATGCGGC GACCGAGTTG CTCTTGCCCG
5701 GCGTCAATAC GGGATAATAC CGCGCCACAT AGCAGAACTT TAAAAGTGCT CATCATTGGA
5761 AAACGTTCTT CGGGGCGAAA ACTCTCAAGG ATCTTACCGC TGTTGAGATC CAGTTCGATG
5821 TAACCCACTC GTGCACCCAA CTGATCTTCA GCATCTTTTA CTTTCACCAG CGTTTCTGGG
5881 TGAGCAAAAA CAGGAAGGCA AAATGCCGCA AAAAAGGGAA TAAGGGCGAC ACGGAAATGT
5941 TGAATACTCA TACTCTTCCT TTTTCAATAT TATTGAAGCA TTTATCAGGG TTATTGTCTC
6001 ATGAGCGGAT ACATATTTGA ATGTATTTAG AAAAATAAAC AAATAGGGGT TCCGCGCACA
6061 TTTCCCCGAA AAGTGCCACC TGACGTCTAA GAAACCATTA TTATCATGAC ATTAACCTAT
6121 AAAAATAGGC GTATCACGAG GCCCTTTCGT CATTTAAATA ATGTATCTAA ACGCAAACTC
6181 CGAGCTGGAA AAATGTTACC GGCGATGCGC GGACAATTTA GAGGCGGCGA TCAAGAAACA
6241 CCTGCTGGGC GAGCAGTCTG GAGCACAGTC TTCGATGGGC CCGAGATCCC ACCGCGTTCC
6301 TGGGTACCGG GACGTGAGGC AGCGCGACAT CCATCAAATA TACCAGGCGC CAACCGAGTG
6361 TCTCGGAAAA CAGCTTCTGG ATATCTTCCG CTGGCGGCGC AACGACGAAT AATAGTCCCT
6421 GGAGGTGACG GAATATATAT GTGTGGAGGG TAAATCTGAC AGGGTGTAGC AAAGGTAATA
6481 TTTTCCTAAA ACATGCAATC GGCTGCCCCG CAACGGGAAA AAGAATGACT TTGGCACTCT
6541 TCACCAGAGT GGGGTGTCCC GCTCGTGTGT GCAAATAGGC TCCCACTGGT CACCCCGGAT
6601 TTTGCAGAAA AACAGCAAGT TCCGGGGTGT CTCACTGGTG TCCGCCAATA AGAGGAGCCG
6661 GCAGGCACGG AGTTTACATC AAGCTGTCTC CGATACACTC GACTACCATC CGGGTCTCTC
6721 AGAGAGGGGA ATGGCACTAT AAATACCGCC TCCTTGCGCT CTCTGCCTTC ATCAATCAAA
6781 TCATGTCTTT TGTCCAAAAG GGTACTTGGT TACTTTTTGC TCTGTTGCAC CCAACTGTTA
6841 TTCTCGCACA ACAGGAAGCA GTAGATGGTG GTTGCTCACA TTTAGGTCAA TCTTACGCAG
6901 ATAGAGATGT ATGGAAACCT GAACCATGTC AAATTTGCGT GTGTGACTCA GGTTCAGTGC
6961 TCTGCGACGA TATCATATGT GACGACCAGG AATTGGACTG TCCAAACCCA GAGATACCAT
7021 TCGGTGAATG TTGTGCTGTT TGTCCACAGC CACCAACTGC TCCTACAAGA CCTCCAAACG
7081 GTCAAGGTCC ACAAGGTCCT AAAGGTGATC CGGGTCCACC TGGTATTCCT GGTAGAAATG
7141 GTGACCCTGG ACCTCCCGGT TCCCCAGGTA GCCCAGGATC ACCTGGGCCT CCTGGAATAT
7201 GTGAATCCTG CCCAACTGGT GGTCAGAACT ATAGCCCACA ATACGAGGCC TACGACGTCA
7261 AATCTGGTGT TGCTGGAGGA GGTATTGCAG GCTACCCTGG TCCCGCAGGG CCCCCAGGTC
7321 CGCCGGGTCC GCCCGGAACA TCAGGTCATC CCGGAGCCCC TGGTGCACCA GGTTATCAGG
7381 GACCGCCCGG AGAGCCTGGA CAAGCTGGTC CCGCTGGACC CCCTGGTCCA CCAGGTGCTA
7441 TTGGACCAAG TGGTCCTGCC GGAAAAGACG GTGAATCCGG TAGACCTGGT AGACCCGGCG
7501 AAAGGGGTTT CCCAGGTCCT CCCGGAATGA AGGGTCCAGC CGGTATGCCC GGTTTTCCTG
7561 GGATGAAGGG TCACAGAGGA TTTGATGGTA GAAACGGAGA GAAAGGCGAA ACCGGTGCTC
7621 CCGGACTGAA GGGTGAAAAC GGTGTCCCTG GTGAGAACGG CGCTCCTGGA CCTATGGGTC
7681 CACGTGGTGC TCCAGGAGAA AGAGGCAGAC CAGGATTGCC TGGTGCAGCT GGTGCTAGAG
7741 GTAACGATGG TGCCCGTGGT TCCGATGGAC AACCCGGGCC ACCCGGCCCT CCAGGTACCG
7801 CTGGATTTCC TGGAAGCCCT GGTGCTAAGG GGGAGGTTGG TCCGGCTGGT AGTCCCGGAA
7861 GTAGCGGTGC CCCAGGTCAA AGAGGCGAAC CAGGCCCTCA GGGTCACGCA GGAGCACCTG
7921 GACCGCCTGG TCCTCCTGGT TCGAATGGTT CGCCTGGAGG AAAAGGTGAA ATGGGGCCCG
7981 CAGGAATCCC CGGTGCGCCT GGTCTTATTG GTGCCAGGGG TCCTCCAGGC CCGCCAGGTA
8041 CAAATGGTGT ACCCGGACAG CGAGGAGCAG CTGGTGAACC TGGTAAAAAC GGTGCCAAAG
8101 GAGATCCAGG TCCTCGTGGA GAGCGTGGTG AAGCTGGCTC TCCCGGTATC GCCGGTCCAA
8161 AAGGTGAGGA CGGTAAGGAC GGTTCCCCTG GTGAGCCAGG TGCGAACGGA CTGCCAGGTG
8221 CAGCCGGAGA GCGAGGAGTC CCAGGATTCA GGGGACCAGC CGGTGCTAAC GGCTTGCCTG
8281 GTGAAAAAGG GCCCCCTGGT GATAGGGGAG GACCCGGTCC AGCAGGCCCT CGTGGAGTTG
8341 CTGGTGAGCC TGGACGTGAC GGTTTACCAG GAGGGCCAGG TTTGAGGGGT ATTCCCGGGT
8401 CCCCTGGCGG TCCTGGATCG GATGGAAAAC CAGGGCCACC AGGTTCGCAG GGTGAAACAG
8461 GACGTCCAGG CCCACCCGGC TCACCTGGTC CAAGGGGTCA GCCTGGTGTC ATGGGTTTCC
8521 CCGGTCCAAA GGGTAATGAC GGAGCACCGG GTAAAAATGG TGAACGTGGT GGCCCAGGTG
8581 GTCCAGGACC CCAAGGTCCA GCTGGAAAAA ACGGTGAGAC AGGTCCTCAA GGACCTCCAG
8641 GACCTACCGG TCCTAGCGGA GATAAGGGAG ATACGGGACC GCCAGGACCT CAAGGATTGC
8701 AAGGTTTGCC TGGTACATCT GGCCCTCCCG GAGAAAATGG TAAGCCTGGA GAGCCAGGAC
8761 CAAAAGGCGA AGCTGGAGCC CCAGGTATCC CCGGAGGTAA GGGAGACTCA GGTGCTCCGG
8821 GTGAGCGTGG TCCTCCGGGT GCCGGTGGTC CACCTGGACC TAGAGGTGGT GCCGGGCCGC
8881 CAGGTCCTGA AGGTGGTAAA GGTGCTGCTG GTCCACCGGG ACCGCCTGGC TCTGCTGGTA
8941 CTCCTGGCTT GCAGGGAATG CCAGGAGAGA GAGGTGGACC TGGAGGTCCC GGTCCGAAGG
9001 GTGATAAAGG GGAGCCAGGA TCATCCGGTG TTGACGGCGC ACCTGGTAAA GACGGACCAA
9061 GGGGACCAAC GGGTCCAATC GGACCACCAG GACCCGCTGG CCAGCCAGGA GATAAAGGCG
9121 AGTCCGGAGC ACCCGGTGTT CCTGGTATAG CTGGACCCAG GGGTGGTCCC GGTGAAAGAG
9181 GTGAACAGGG CCCACCGGGT CCCGCCGGTT TCCCTGGCGC CCCTGGTCAA AATGGAGAAC
9241 CAGGTGCAAA GGGCGAGAGA GGAGCCCCAG GAGAAAAGGG TGAGGGAGGA CCACCCGGTG
9301 CTGCCGGTCC AGCTGGGGGT TCAGGTCCTG CTGGACCACC AGGTCCACAG GGCGTTAAAG
9361 GTGAGAGAGG AAGTCCAGGT GGTCCTGGAG CTGCTGGATT CCCAGGTGGC CGTGGACCTC
9421 CTGGTCCCCC TGGATCGAAT GGTAATCCTG GTCCGCCAGG TAGTTCGGGT GCTCCTGGGA
9481 AGGACGGTCC ACCTGGCCCC CCAGGTAGTA ACGGTGCACC TGGTAGTCCA GGTATATCCG
9541 GACCTAAAGG AGATTCCGGT CCACCAGGCG AAAGAGGGGC CCCAGGCCCA CAGGGTCCAC
9601 CAGGAGCCCC CGGTCCTCTG GGTATTGCTG GTCTTACTGG TGCACGTGGA CTGGCCGGTC
9661 CACCCGGAAT GCCTGGAGCA AGAGGTTCAC CTGGACCACA AGGTATTAAA GGAGAGAACG
9721 GTAAACCTGG ACCTTCCGGT CAAAACGGAG AGCGGGGACC CCCAGGCCCC CAAGGTCTGC
9781 CAGGACTAGC TGGTACCGCA GGGGAACCAG GAAGAGATGG AAATCCAGGT TCAGACGGAC
9841 TACCCGGTAG AGATGGTGCA CCGGGGGCCA AGGGCGACAG GGGTGAGAAT GGATCTCCTG
9901 GTGCGCCAGG GGCACCAGGC CACCCAGGTC CCCCAGGTCC TGTGGGCCCT GCTGGAAAGT
9961 CAGGTGACAG GGGAGAGACA GGCCCGGCTG GTCCATCTGG CGCACCCGGA CCAGCTGGTT
10021 CCAGAGGCCC ACCTGGTCCG CAAGGCCCTA GAGGTGACAA GGGAGAGACT GGAGAACGAG
10081 GTGCTATGGG TATCAAGGGT CATAGAGGTT TTCCGGGTAA TCCCGGCGCC CCAGGTTCTC
10141 CTGGTCCAGC TGGCCATCAA GGTGCAGTCG GATCGCCCGG CCCAGCCGGT CCCAGGGGCC
10201 CTGTTGGTCC ATCCGGTCCT CCAGGAAAGG ATGGTGCTTC TGGACACCCA GGACCTATCG
10261 GACCTCCGGG TCCTAGAGGT AATAGAGGAG AACGTGGATC CGAGGGTAGT CCTGGTCACC
10321 CTGGTCAACC TGGCCCACCA GGGCCTCCAG GTGCACCCGG TCCATGTTGT GGTGCAGGCG
10381 GTGTGGCTGC AATTGCTGGT GTGGGTGCTG AAAAGGCCGG CGGTTTCGCT CCATATTATG
10441 GTTAAGGCGG CCGCAAACG

SEQ ID NO 12: MMV-644

1 GGATCCTTCA GTAATGTCTT GTTTCTTTTG TTGCAGTGGT GAGCCATTTT GACTTCGTGA
61 AAGTTTCTTT AGAATAGTTG TTTCCAGAGG CCAAACATTC CACCCGTAGT AAAGTGCAAG
121 CGTAGGAAGA CCAAGACTGG CATAAATCAG GTATAAGTGT CGAGCACTGG CAGGTGATCT
181 TCTGAAAGTT TCTACTAGCA GATAAGATCC AGTAGTCATG CATATGGCAA CAATGTACCG
241 TGTGGATCTA AGAACGCGTC CTACTAACCT TCGCATTCGT TGGTCCAGTT TGTTGTTATC
301 GATCAACGTG ACAAGGTTGT CGATTCCGCG TAAGCATGCA TACCCAAGGA CGCCTGTTGC
361 AATTCCAAGT GAGCCAGTTC CAACAATCTT TGTAATATTA GAGCACTTCA TTGTGTTGCG
421 CTTGAAAGTA AAATGCGAAC AAATTAAGAG ATAATCTCGA AACCGCGACT TCAAACGCCA
481 ATATGATGTG CGGCACACAA TAAGCGTTCA TATCCGCTGG GTGACTTTCT CGCTTTAAAA
541 AATTATCCGA AAAAATTTTC TAGAGTGTTG TTACTTTATA CTTCCGGCTC GTATAATACG
601 ACAAGGTGTA AGGAGGACTA AACCATGGCT AAACTCACCT CTGCTGTTCC AGTCCTGACT
661 GCTCGTGATG TTGCTGGTGC TGTTGAGTTC TGGACTGATA GGCTCGGTTT CTCCCGTGAC
721 TTCGTAGAGG ACGACTTTGC CGGTGTTGTA CGTGACGACG TTACCCTGTT CATCTCCGCA
781 GTTCAGGACC AGGTTGTGCC AGACAACACT CTGGCATGGG TATGGGTTCG TGGTCTGGAC
841 GAACTGTACG CTGAGTGGTC TGAGGTCGTG TCTACCAACT TCCGTGATGC ATCTGGTCCA
901 GCTATGACCG AGATCGGTGA ACAGCCCTGG GGTCGTGAGT TTGCACTGCG TGATCCAGCT
961 GGTAACTGCG TGCATTTCGT CGCAGAAGAG CAGGACTAAC AATTGACACC TTACGATTAT
1021 TTAGAGAGTA TTTATTAGTT TTATTGTATG TATACGGATG TTTTATTATC TATTTATGCC
1081 CTTATATTCT GTAACTATCC AAAAGTCCTA TCTTATCAAG CCAGCAATCT ATGTCCGCGA
1141 ACGTCAACTA AAAATAAGCT TTTTATGCTC TTCTCTCTTT TTTTCCCTTC GGTATAATTA
1201 TACCTTGCAT CCACAGATTC TCCTGCCAAA TTTTGCATAA TCCTTTACAA CATGGCTATA
1261 TGGGAGCACT TAGCGCCCTC CAAAACCCAT ATTGCCTACG CATGTATAGG TGTTTTTTCC
1321 ACAATATTTT CTCTGTGCTC TCTTTTTATT AAAGAGAAGC TCTATATCGG AGAAGCTTCT
1381 GTGGCCGTTA TATTCGGCCT TATCGTGGGA CCACATTGCC TGAATTGGTT TGCCCCGGAA
1441 GATTGGGGAA ACTTGGATCT GATTACCTTA GCTGCAGAAA AGGGTACCAC TGAGCGTCAG
1501 ACCCCGTAGA AAAGATCAAA GGATCTTCTT GAGATCCTTT TTTTCTGCGC GTAATCTGCT
1561 GCTTGCAAAC AAAAAAACCA CCGCTACCAG CGGTGGTTTG TTTGCCGGAT CAAGAGCTAC
1621 CAACTCTTTT TCCGAAGGTA ACTGGCTTCA GCAGAGCGCA GATACCAAAT ACTGTTCTTC
1681 TAGTGTAGCC GTAGTTAGGC CACCACTTCA AGAACTCTGT AGCACCGCCT ACATACCTCG
1741 CTCTGCTAAT CCTGTTACCA GTGGCTGCTG CCAGTGGCGA TAAGTCGTGT CTTACCGGGT
1801 TGGACCCAAG ACGATAGTTA CCGGATAAGG CGCAGCGGTC GGGCTGAACG GGGGGTTCGT
1861 GCACACAGCC CAGCTTGGAG CGAACGACCT ACACCGAACT GAGATACCTA CAGCGTGAGC
1921 TATGAGAAAG CGCCACGCTT CCCGAAGGGA GAAAGGCGGA CAGGTATCCG GTAAGCGGCA
1981 GGGTCGGAAC AGGAGAGCGC ACGAGGGAGC TTCCAGGGGG AAACGCCTGG TATCTTTATA
2041 GTCCTGTCGG GTTTCGCCAC CTCTGACTTG AGCGTCGATT TTTGTGATGC TCGTCAGGGG
2101 GGCGGAGCCT ATGGAAAAAC GCCAGCAACG CGGCCTTTTT ACGGTTCCTG GCCTTTTGCT
2161 GGCCTTTTGC TCACATGTAT TTAAATAATG TATCTAAACG CAAACTCCGA GCTGGAAAAA
2221 TGTTACCGGC GATGCGCGGA CAATTTAGAG GCGGCGATCA AGAAACACCT GCTGGGCGAG
2281 CAGTCTGGAG CACAGTCTTC GATGGGCCCG AGATCCCACC GCGTTCCTGG GTACCGGGAC
2341 GTGAGGCAGC GCGACATCCA TCAAATATAC CAGGCGCCAA CCGAGTGTCT CGGAAAACAG
2401 CTTCTGGATA TCTTCCGCTG GCGGCGCAAC GACGAATAAT AGTCCCTGGA GGTGACGGAA
2461 TATATATGTG TGGAGGGTAA ATCTGACAGG GTGTAGCAAA GGTAATATTT TCCTAAAACA
2521 TGCAATCGGC TGCCCCGCAA CGGGAAAAAG AATGACTTTG GCACTCTTCA CCAGAGTGGG
2581 GTGTCCCGCT CGTGTGTGCA AATAGGCTCC CACTGGTCAC CCCGGATTTT GCAGAAAAAC
2641 AGCAAGTTCC GGGGTGTCTC ACTGGTGTCC GCCAATAAGA GGAGCCGGCA GGCACGGAGT
2701 TTACATCAAG CTGTCTCCGA TACACTCGAC TACCATCCGG GTCTCTCAGA GAGGGGAATG
2761 GCACTATAAA TACCGCCTCC TTGCGCTCTC TGCCTTCATC AATCAAATCA TGAGATTCCC
2821 ATCTATTTTC ACCGCTGTCT TGTTCGCTGC CTCCTCTGCA TTGGCTGCCC CTGTTAACAC
2881 TACCACTGAA GACGAGACTG CTCAAATTCC AGCTGAAGCA GTTATCGGTT ACTCTGACCT
2941 TGAGGGTGAT TTCGACGTCG CTGTTTTGCC TTTCTCTAAC TCCACTAACA ACGGTTTGTT
3001 GTTCATTAAC ACCACTATCG CTTCCATTGC TGCTAAGGAA GAGGGTGTCT CTCTCGAGAA
3061 AAGAGAGGCC GAAGCTGTGC TGTCAAAGTC CTGTGTCAGT CACTTTAGAA ATGTTGGATC
3121 CTTGAATAGT AGGGATGTCA ATCTGAAAGA TGACTTTTCC TATGCTAATA TTGATGATCC
3181 CTATAACAAG CCTTTCGTCC TAAATAACCT AATAAACCCT ACCAAGTGTC AAGAGATCAT
3241 GCAATTTGCC AATGGCAAGT TGTTTGACTC CCAAGTCCTG AGTGGCACGG ACAAGAACAT
3301 ACGTAACTCT CAACAAATGT GGATATCCAA GAACAACCCT ATGGTAAAAC CCATTTTCGA
3361 GAACATATGC AGGCAGTTTA ACGTACCCTT TGATAATGCC GAGGACCTAC AGGTCGTCCG
3421 TTACTTGCCT AATCAATATT ATAATGAGCA TCATGACTCA TGCTGTGACT CCTCCAAGCA
3481 ATGCAGTGAA TTTATAGAGA GGGGCGGTCA GAGGATTCTG ACCGTTTTAA TTTACCTAAA
3541 CAACGAGTTC TCAGATGGAC ACACGTACTT TCCTAATTTA AACCAAAAGT TCAAGCCCAA
3601 GACTGGTGAT GCTTTGGTTT TTTACCCTTT AGCCAACAAC TCTAATAAAT GTCACCCATA
3661 CAGTCTACAC GCAGGTATGC CCGTCACGTC AGGAGAGAAG TGGATTGCTA ATCTGTGGTT
3721 TCGTGAGCGT AAGTTCTCCC ACCACCACCA CCACCACTAA TAATCAAGAG GATGTCAGAA
3781 TGCCATTTGC CTGAGAGATG CAGGCTTCAT TTTTGATACT TTTTTATTTG TAACCTATAT
3841 AGTATAGGAT TTTTTTTGTC ATTTTGTTTC TTCTCGTACG AGCTTGCTCC TGATCAGCCT
3901 ATCTCGCAGC TGATGAATAT CTTGTGGTAG GGGTTTGGGA AAATCATTCG AGTTTGATGT
3961 TTTTCTTGGT ATTTCCCACT CCTCTTCAGA GTACAGAAGA TTAAGTGAGA CGTTCGTTTG
4021 TGCTCCGGA

SEQ ID NO 13: MMV-580

1 GGATCCTTCA GTAATGTCTT GTTTCTTTTG TTGCAGTGGT GAGCCATTTT GACTTCGTGA
61 AAGTTTCTTT AGAATAGTTG TTTCCAGAGG CCAAACATTC CACCCGTAGT AAAGTGCAAG
121 CGTAGGAAGA CCAAGACTGG CATAAATCAG GTATAAGTGT CGAGCACTGG CAGGTGATCT
181 TCTGAAAGTT TCTACTAGCA GATAAGATCC AGTAGTCATG CATATGGCAA CAATGTACCG
241 TGTGGATCTA AGAACGCGTC CTACTAACCT TCGCATTCGT TGGTCCAGTT TGTTGTTATC
301 GATCAACGTG ACAAGGTTGT CGATTCCGCG TAAGCATGCA TACCCAAGGA CGCCTGTTGC
361 AATTCCAAGT GAGCCAGTTC CAACAATCTT TGTAATATTA GAGCACTTCA TTGTGTTGCG
421 CTTGAAAGTA AAATGCGAAC AAATTAAGAG ATAATCTCGA AACCGCGACT TCAAACGCCA
481 ATATGATGTG CGGCACACAA TAAGCGTTCA TATCCGCTGG GTGACTTTCT CGCTTTAAAA
541 AATTATCCGA AAAAATTTTC CTCTAGAATG GGTAAGGAAA AGACTCACGT TTCGAGGCCG
601 CGATTAAATT CCAACATGGA TGCTGATTTA TATGGGTATA AATGGGCTCG CGATAATGTC
661 GGGCAATCAG GTGCGACAAT CTATCGATTG TATGGGAAGC CCGATGCGCC AGAGTTGTTT
721 CTGAAACATG GCAAAGGTAG CGTTGCCAAT GATGTTACAG ATGAGATGGT CAGACTAAAC
781 TGGCTGACGG AATTTATGCC TCTTCCGACC ATCAAGCATT TTATCCGTAC TCCTGATGAT
841 GCATGGTTAC TCACCACTGC GATCCCCGGC AAAACAGCAT TCCAGGTATT AGAAGAATAT
901 CCTGATTCAG GTGAAAATAT TGTTGATGCG CTGGCAGTGT TCCTGCGCCG GTTGCATTCG
961 ATTCCTGTTT GTAATTGTCC TTTTAACAGC GATCGCGTAT TTCGTCTCGC TCAGGCGCAA
1021 TCACGAATGA ATAACGGTTT GGTTGATGCG AGTGATTTTG ATGACGAGCG TAATGGCTGG
1081 CCTGTTGAAC AAGTCTGGAA AGAAATGCAT AAGCTTTTGC CATTCTCACC GGATTCAGTC
1141 GTCACTCATG GTGATTTCTC ACTTGATAAC CTTATTTTTG ACGAGGGGAA ATTAATAGGT
1201 TGTATTGATG TTGGACGAGT CGGAATCGCA GACCGATACC AGGATCTTGC CATCCTATGG
1261 AACTGCCTCG GTGAGTTTTC TCCTTCATTA CAGAAACGGC TTTTTCAAAA ATATGGTATT
1321 GATAATCCTG ATATGAATAA ATTGCAGTTT CATTTGATGC TCGATGAGTT TTTCTAAAAT
1381 TGACACCTTA CGATTATTTA GAGAGTATTT ATTAGTTTTA TTGTATGTAT ACGGATGTTT
1441 TATTATCTAT TTATGCCCTT ATATTCTGTA ACTATCCAAA AGTCCTATCT TATCAAGCCA
1501 GCAATCTATG TCCGCGAACG TCAACTAAAA ATAAGCTTTT TATGCTGTTC TCTCTTTTTT
1561 TCCCTTCGGT ATAATTATAC CTTGCATCCA CAGATTCTCC TGCCAAATTT TGCATAATCC
1621 TTTACAACAT GGCTATATGG GAGCACTTAG CGCCCTCCAA AACCCATATT GCCTACGCAT
1681 GTATAGGTGT TTTTTCCACA ATATTTTCTC TGTGCTCTCT TTTTATTAAA GAGAAGCTCT
1741 ATATCGGAGA AGCTTCTGTG GCCGTTATAT TCGGCCTTAT CGTGGGACCA CATTGCCTGA
1801 ATTGGTTTGC CCCGGAAGAT TGGGGAAACT TGGATCTGAT TACCTTAGCT GCATTACCAA
1861 TGCTTAATCA GTGAGGCACC TATCTCAGCG ATCTGTCTAT TTCGTTCATC CATAGTTGCC
1921 TGACTCCCCG TCGTGTAGAT AACTACGATA CGGGAGGGCT TACCATCTGG CCCCAGCGCT
1981 GCGATGATAC CGCGAGAACC ACGCTCACCG GCTCCGGATT TATCAGCAAT AAACCAGCCA
2041 GCCGGAAGGG CCGAGCGCAG AAGTGGTCCT GCAACTTTAT CCGCCTCCAT CCAGTCTATT
2101 AATTGTTGCC GGGAAGCTAG AGTAAGTAGT TCGCCAGTTA ATAGTTTGCG CAACGTTGTT
2161 GCCATCGCTA CAGGCATCGT GGTGTCACGC TCGTCGTTTG GTATGGCTTC ATTCAGCTCC
2221 GGTTCCCAAC GATCAAGGCG AGTTACATGA TCCCCCATGT TGTGCAAAAA AGCGGTTAGC
2281 TCCTTCGGTC CTCCGATCGT TGTCAGAAGT AAGTTGGCCG CAGTGTTATC ACTCATGGTT
2341 ATGGCAGCAC TGCATAATTC TCTTACTGTC ATGCCATCCG TAAGATGCTT TTCTGTGACT
2401 GGTGAGTACT CAACCAAGTC ATTCTGAGAA TAGTGTATGC GGCGACCGAG TTGCTCTTGC
2461 CCGGCGTCAA TACGGGATAA TACCGCGCCA CATAGCAGAA CTTTAAAAGT GCTCATCATT
2521 GGAAAACGTT CTTCGGGGCG AAAACTCTCA AGGATCTTAC CGCTGTTGAG ATCCAGTTCG
2581 ATGTAACCCA CTCGTGCACC CAACTGATCT TCAGCATCTT TTACTTTCAC CAGCGTTTCT
2641 GGGTGAGCAA AAACAGGAAG GCAAAATGCC GCAAAAAAGG GAATAAGGGC GACACGGAAA
2701 TGTTGAATAC TCATATTCTT CCTTTTTCAA TATTATTGAA GCATTTATCA GGGTTATTGT
2761 CTCATGAGCG GATACATATT TGAATGTATT TAGAAAAATA AACAAATAGG GGTCAGTGTT
2821 ACAACCAATT AACCAATTCT GAAAGGAAGA ATCTGCAGGA AAAGGGTACC ACTGAGCGTC
2881 AGACCCCGTA GAAAAGATCA AAGGATCTTC TTGAGATCCT TTTTTTCTGC GCGTAATCTG
2941 CTGCTTGCAA ACAAAAAAAC CACCGCTACC AGCGGTGGTT TGTTTGCCGG ATCAAGAGCT
3001 ACCAACTCTT TTTCCGAAGG TAACTGGCTT CAGCAGAGCG CAGATACCAA ATACTGTTCT
3061 TCTAGTGTAG CCGTAGTTAG GCCACCACTT CAAGAACTCT GTAGCACCGC CTACATACCT
3121 CGCTCTGCTA ATCCTGTTAC CAGTGGCTGC TGCCAGTGGC GATAAGTCGT GTCTTACCGG
3181 GTTGGACCCA AGACGATAGT TACCGGATAA GGCGCAGCGG TCGGGCTGAA CGGGGGGTTC
3241 GTGCACACAG CCCAGCTTGG AGCGAACGAC CTACACCGAA CTGAGATACC TACAGCGTGA
3301 GCTATGAGAA AGCGCCACGC TTCCCGAAGG GAGAAAGGCG GACAGGTATC CGGTAAGCGG
3361 CAGGGTCGGA ACAGGAGAGC GCACGAGGGA GCTTCCAGGG GGAAACGCCT GGTATCTTTA
3421 TAGTCCTGTC GGGTTTCGCC ACCTCTGACT TGAGCGTCGA TTTTTGTGAT GCTCGTCAGG
3481 GGGGCGGAGC CTATGGAAAA ACGCCAGCAA CGCGGCCTTT TTACGGTTCC TGGCCTTTTG
3541 CTGGCCTTTT GCTCACATGT TTTGTTCGAT TATTCTCCAG ATAAAATCAA CAATAGTTGT
3601 TTGTAAGTAA ACGAATCAAG ATACTGAAAA TAGTTTCAAA AGCAGATCAT CTGGGATTTA
3661 TATATCAGGC ATCCTGCTTT AGTTCTTTTT TGAACCCAAA GGCTATCTGA TGAAAAGTTG
3721 ATATAGGTAT GAAGACCAGA ATTTGCCTAG AGGCTAACCG AGACCTGAGG CTAAAAAAGG
3781 CAGGAGGAAA AGTCCTGCCA AAGATAGGTA TTTGAACTTG TTCGAAAAAG GCGGAAgttt
3841 aaacACATGG TTGGAGCAAG CGGCGGAATA GCGGAGGGAT GATACGCAGC AAGGCTGGGA
3901 TCATTCGAGT TTCAAGGAAC GTTAGCTCAA CATTCATTGA CTGGTAAGCG ACAACTGGTT
3961 TCATCTGGGT GGAGTTAGTC TGGTGTTGGG ATGCTAGTTG TTCCCCACAA TTGAAGGCCA
4021 GATGAGGAGG ATGGTGTGGT GATAAGAGAT GCAAACAGAT GGTTATGGCC TTTTGAGAAC
4081 AAAGTAGACC TGTCACTCAA TTGTTGTTTA TATCATTGCT ATTTAAATCA GGTGAACCCA
4141 CCTAACTATT TTTAACTGGC ATCCAGTGAG CTCGCTGGGT GAAAGCCAAC CATCTTTTGT
4201 TTCGGGGAAC CGTGCTCGCC CCGTAAAGTT AATTTTTTTT TCCCGCGCAG CTTTAATCTT
4261 TCGGCAGAGA AGGCGTTTTC ATCGTAGCGT GGGAACAGAA TAATCAGTTC ATGTGCTATA
4321 CAGGCACATG GCAGCAGTCA CTATTTTGCT TTTTAACCTT AAAGTCGTTC ATCAATCATT
4381 AACTGACCAA TCAGATTTTT TGCATTTGCC ACTTATCTAA AAATACTTTT GTATCTCGCA
4441 GATACGTTCA GTGGTTTCCA GGACAACACC CAAAAAAAGG TATCAATGCC ACTAGGCAGT
4501 CGGTTTTATT TTTGGTCACC CACGCAAAGA AGCACCCACC TCTTTTAGGT TTTAAGTTGT
4561 GGGAACAGTA ACACCGCCTA GAGCTTCAGG AAAAACCAGT ACCTGTGACC GCAATTCACC
4621 ATGATGCAGA ATGTTAATTT AAACGAGTGC CAAATCAAGA TTTCAACAGA CAAATCAATC
4681 GATCCATAGT TACCCATTCC AGCCTTTTCG TCGTCGAGCC TGCTTCATTC CTGCCTCAGG
4741 TGCATAACTT TGCATGAAAA GTCCAGATTA GGGCAGATTT TGAGTTTAAA ATAGGAAATA
4801 TAAACAAATA TACCGCGAAA AAGGTTTGTT TATAGCTTTT CGCCTGGTGC CGTACGGTAT
4861 AAATACATAC TCTCCTCCCC CCCCTGGTTC TCTTTTTCTT TTGTTACTTA CATTTTACCG
4921 TTCCGTCACT CGCTTCACTC AACAACAAAA ATGTTCTCTC CAATTTTGTC CTTGGAAATT
4981 ATTTTAGCTT TGGCTACTTT GCAATCTGTC TTCGCTGTGC TGTCAAAGTC CTGTGTCAGT
5041 CACTTTAGAA ATGTTGGATC CTTGAATAGT AGGGATGTCA ATCTGAAAGA TGACTTTTCC
5101 TATGCTAATA TTGATGATCC CTATAACAAG CCTTTCGTCC TAAATAACCT AATAAACCCT
5161 ACCAAGTGTC AAGAGATCAT GCAATTTGCC AATGGCAAGT TGTTTGACTC CCAAGTCCTG
5221 AGTGGCACGG ACAAGAACAT ACGTAACTCT CAACAAATGT GGATATCCAA GAACAACCCT
5281 ATGGTAAAAC CCATTTTCGA GAACATATGC AGGCAGTTTA ACGTACCCTT TGATAATGCC
5341 GAGGACCTAC AGGTCGTCCG TTACTTGCCT AATCAATATT ATAATGAGCA TCATGACTCA
5401 TGCTGTGACT CCTCCAAGCA ATGCAGTGAA TTTATAGAGA GGGGCGGTCA GAGGATTCTG
5461 ACCGTTTTAA TTTACCTAAA CAACGAGTTC TCAGATGGAC ACACGTACTT TCCTAATTTA
5521 AACCAAAAGT TCAAGCCCAA GACTGGTGAT GCTTTGGTTT TTTACCCTTT AGCCAACAAC
5581 TCTAATAAAT GTCACCCATA CAGTCTACAC GCAGGTATGC CCGTCACGTC AGGAGAGAAG
5641 TGGATTGCTA ATCTGTGGTT TCGTGAGCGT AAGTTCTCCC ACCACCACCA CCACCACTAA
5701 TGAAGATCTG GAGGAGGCTG AGGAACCTGA TCTTGAGGAG GATGACGACC AGAAGGCAGT
5761 CAAAGATGAA CTGTGATAAG GGGGGCCGCG AGTCGTGAGT AATCAAGAGG ATGTCAGAAT
5821 GCCATTTGCC TGAGAGATGC AGGCTTCATT TTTGATACTT TTTTATTTGT AACCTATATA
5881 GTATAGGATT TTTTTTGTCA TTTTGTTTCT TCTCGTACGA GCTTGCTCCT GATCAGCCTA
5941 TCTCGCAGCT GATGAATATC TTGTGGTAGG GGTTTGGGAA AATCATTCGA GTTTGATGTT
6001 TTTCTTGGTA TTTCCCACTC CTCTTCAGAG TACAGAAGAT TAAGTGAGAC GTTCGTTTGT
6061 GCTCCGGA

SEQ ID NO 14: MMV-630

1 GGATCCTTCA GTAATGTCTT GTTTCTTTTG TTGCAGTGGT GAGCCATTTT GACTTCGTGA
61 AAGTTTCTTT AGAATAGTTG TTTCCAGAGG CCAAACATTC CACCCGTAGT AAAGTGCAAG
121 CGTAGGAAGA CCAAGACTGG CATAAATCAG GTATAAGTGT CGAGCACTGG CAGGTGATCT
181 TCTGAAAGTT TCTACTAGCA GATAAGATCC AGTAGTCATG CATATGGCAA CAATGTACCG
241 TGTGGATCTA AGAACGCGTC CTACTAACCT TCGCATTCGT TGGTCCAGTT TGTTGTTATC
301 GATCAACGTG ACAAGGTTGT CGATTCCGCG TAAGCATGCA TACCCAAGGA CGCCTGTTGC
361 AATTCCAAGT GAGCCAGTTC CAACAATCTT TGTAATATTA GAGCACTTCA TTGTGTTGCG
421 CTTGAAAGTA AAATGCGAAC AAATTAAGAG ATAATCTCGA AACCGCGACT TCAAACGCCA
481 ATATGATGTG CGGCACACAA TAAGCGTTCA TATCCGCTGG GTGACTTTCT CGCTTTAAAA
541 AATTATCCGA AAAAATTTTC CTCTAGAATG GGTAAGGAAA AGACTCACGT TTCGAGGCCG
601 CGATTAAATT CCAACATGGA TGCTGATTTA TATGGGTATA AATGGGCTCG CGATAATGTC
661 GGGCAATCAG GTGCGACAAT CTATCGATTG TATGGGAAGC CCGATGCGCC AGAGTTGTTT
721 CTGAAACATG GCAAAGGTAG CGTTGCCAAT GATGTTACAG ATGAGATGGT CAGACTAAAC
781 TGGCTGACGG AATTTATGCC TCTTCCGACC ATCAAGCATT TTATCCGTAC TCCTGATGAT
841 GCATGGTTAC TCACCACTGC GATCCCCGGC AAAACAGCAT TCCAGGTATT AGAAGAATAT
901 CCTGATTCAG GTGAAAATAT TGTTGATGCG CTGGCAGTGT TCCTGCGCCG GTTGCATTCG
961 ATTCCTGTTT GTAATTGTCC TTTTAACAGC GATCGCGTAT TTCGTCTCGC TCAGGCGCAA
1021 TCACGAATGA ATAACGGTTT GGTTGATGCG AGTGATTTTG ATGACGAGCG TAATGGCTGG
1081 CCTGTTGAAC AAGTCTGGAA AGAAATGCAT AAGCTTTTGC CATTCTCACC GGATTCAGTC
1141 GTCACTCATG GTGATTTCTC ACTTGATAAC CTTATTTTTG ACGAGGGGAA ATTAATAGGT
1201 TGTATTGATG TTGGACGAGT CGGAATCGCA GACCGATACC AGGATCTTGC CATCCTATGG
1261 AACTGCCTCG GTGAGTTTTC TCCTTCATTA CAGAAACGGC TTTTTCAAAA ATATGGTATT
1321 GATAATCCTG ATATGAATAA ATTGCAGTTT CATTTGATGC TCGATGAGTT TTTCTAAAAT
1381 TGACACCTTA CGATTATTTA GAGAGTATTT ATTAGTTTTA TTGTATGTAT ACGGATGTTT
1441 TATTATCTAT TTATGCCCTT ATATTCTGTA ACTATCCAAA AGTCCTATCT TATCAAGCCA
1501 GCAATCTATG TCCGCGAACG TCAACTAAAA ATAAGCTTTT TATGCTGTTC TCTCTTTTTT
1561 TCCCTTCGGT ATAATTATAC CTTGCATCCA CAGATTCTCC TGCCAAATTT TGCATAATCC
1621 TTTACAACAT GGCTATATGG GAGCACTTAG CGCCCTCCAA AACCCATATT GCCTACGCAT
1681 GTATAGGTGT TTTTTCCACA ATATTTTCTC TGTGCTCTCT TTTTATTAAA GAGAAGCTCT
1741 ATATCGGAGA AGCTTCTGTG GCCGTTATAT TCGGCCTTAT CGTGGGACCA CATTGCCTGA
1801 ATTGGTTTGC CCCGGAAGAT TGGGGAAACT TGGATCTGAT TACCTTAGCT GCATTACCAA
1861 TGCTTAATCA GTGAGGCACC TATCTCAGCG ATCTGTCTAT TTCGTTCATC CATAGTTGCC
1921 TGACTCCCCG TCGTGTAGAT AACTACGATA CGGGAGGGCT TACCATCTGG CCCCAGCGCT
1981 GCGATGATAC CGCGAGAACC ACGCTCACCG GCTCCGGATT TATCAGCAAT AAACCAGCCA
2041 GCCGGAAGGG CCGAGCGCAG AAGTGGTCCT GCAACTTTAT CCGCCTCCAT CCAGTCTATT
2101 AATTGTTGCC GGGAAGCTAG AGTAAGTAGT TCGCCAGTTA ATAGTTTGCG CAACGTTGTT
2161 GCCATCGCTA CAGGCATCGT GGTGTCACGC TCGTCGTTTG GTATGGCTTC ATTCAGCTCC
2221 GGTTCCCAAC GATCAAGGCG AGTTACATGA TCCCCCATGT TGTGCAAAAA AGCGGTTAGC
2281 TCCTTCGGTC CTCCGATCGT TGTCAGAAGT AAGTTGGCCG CAGTGTTATC ACTCATGGTT
2341 ATGGCAGCAC TGCATAATTC TCTTACTGTC ATGCCATCCG TAAGATGCTT TTCTGTGACT
2401 GGTGAGTACT CAACCAAGTC ATTCTGAGAA TAGTGTATGC GGCGACCGAG TTGCTCTTGC
2461 CCGGCGTCAA TACGGGATAA TACCGCGCCA CATAGCAGAA CTTTAAAAGT GCTCATCATT
2521 GGAAAACGTT CTTCGGGGCG AAAACTCTCA AGGATCTTAC CGCTGTTGAG ATCCAGTTCG
2581 ATGTAACCCA CTCGTGCACC CAACTGATCT TCAGCATCTT TTACTTTCAC CAGCGTTTCT
2641 GGGTGAGCAA AAACAGGAAG GCAAAATGCC GCAAAAAAGG GAATAAGGGC GACACGGAAA
2701 TGTTGAATAC TCATATTCTT CCTTTTTCAA TATTATTGAA GCATTTATCA GGGTTATTGT
2761 CTCATGAGCG GATACATATT TGAATGTATT TAGAAAAATA AACAAATAGG GGTCAGTGTT
2821 ACAACCAATT AACCAATTCT GAAAGGAAGA ATCTGCAGGA AAAGGGTACC ACTGAGCGTC
2881 AGACCCCGTA GAAAAGATCA AAGGATCTTC TTGAGATCCT TTTTTTCTGC GCGTAATCTG
2941 CTGCTTGCAA ACAAAAAAAC CACCGCTACC AGCGGTGGTT TGTTTGCCGG ATCAAGAGCT
3001 ACCAACTCTT TTTCCGAAGG TAACTGGCTT CAGCAGAGCG CAGATACCAA ATACTGTTCT
3061 TCTAGTGTAG CCGTAGTTAG GCCACCACTT CAAGAACTCT GTAGCACCGC CTACATACCT
3121 CGCTCTGCTA ATCCTGTTAC CAGTGGCTGC TGCCAGTGGC GATAAGTCGT GTCTTACCGG
3181 GTTGGACCCA AGACGATAGT TACCGGATAA GGCGCAGCGG TCGGGCTGAA CGGGGGGTTC
3241 GTGCACACAG CCCAGCTTGG AGCGAACGAC CTACACCGAA CTGAGATACC TACAGCGTGA
3301 GCTATGAGAA AGCGCCACGC TTCCCGAAGG GAGAAAGGCG GACAGGTATC CGGTAAGCGG
3361 CAGGGTCGGA ACAGGAGAGC GCACGAGGGA GCTTCCAGGG GGAAACGCCT GGTATCTTTA
3421 TAGTCCTGTC GGGTTTCGCC ACCTCTGACT TGAGCGTCGA TTTTTGTGAT GCTCGTCAGG
3481 GGGGCGGAGC CTATGGAAAA ACGCCAGCAA CGCGGCCTTT TTACGGTTCC TGGCCTTTTG
3541 CTGGCCTTTT GCTCACATGT TTTGTTCGAT TATTCTCCAG ATAAAATCAA CAATAGTTGT
3601 TTGTAAGTAA ACGAATCAAG ATACTGAAAA TAGTTTCAAA AGCAGATCAT CTGGGATTTA
3661 TATATCAGGC ATCCTGCTTT AGTTCTTTTT TGAACCCAAA GGCTATCTGA TGAAAAGTTG
3721 ATATAGGTAT GAAGACCAGA ATTTGCCTAG AGGCTAACCG AGACCTGAGG CTAAAAAAGG
3781 CAGGAGGAAA AGTCCTGCCA AAGATAGGTA TTTGAACTTG TTCGAAAAAG GCGGAAgttt
3841 aaacACATGG TTGGAGCAAG CGGCGGAATA GCGGAGGGAT GATACGCAGC AAGGCTGGGA
3901 TCATTCGAGT TTCAAGGAAC GTTAGCTCAA CATTCATTGA CTGGTAAGCG ACAACTGGTT
3961 TCATCTGGGT GGAGTTAGTC TGGTGTTGGG ATGCTAGTTG TTCCCCACAA TTGAAGGCCA
4021 GATGAGGAGG ATGGTGTGGT GATAAGAGAT GCAAACAGAT GGTTATGGCC TTTTGAGAAC
4081 AAAGTAGACC TGTCACTCAA TTGTTGTTTA TATCATTGCT ATTTAAATAA TGTATCTAAA
4141 CGCAAACTCC GAGCTGGAAA AATGTTACCG GCGATGCGCG GACAATTTAG AGGCGGCGAT
4201 CAAGAAACAC CTGCTGGGCG AGCAGTCTGG AGCACAGTCT TCGATGGGCC CGAGATCCCA
4261 CCGCGTTCCT GGGTACCGGG ACGTGAGGCA GCGCGACATC CATCAAATAT ACCAGGCGCC
4321 AACCGAGTGT CTCGGAAAAC AGCTTCTGGA TATCTTCCGC TGGCGGCGCA ACGACGAATA
4381 ATAGTCCCTG GAGGTGACGG AATATATATG TGTGGAGGGT AAATCTGACA GGGTGTAGCA
4441 AAGGTAATAT TTTCCTAAAA CATGCAATCG GCTGCCCCGC AACGGGAAAA AGAATGACTT
4501 TGGCACTCTT CACCAGAGTG GGGTGTCCCG CTCGTGTGTG CAAATAGGCT CCCACTGGTC
4561 ACCCCGGATT TTGCAGAAAA ACAGCAAGTT CCGGGGTGTC TCACTGGTGT CCGCCAATAA
4621 GAGGAGCCGG CAGGCACGGA GTTTACATCA AGCTGTCTCC GATACACTCG ACTACCATCC
4681 GGGTCTCTCA GAGAGGGGAA TGGCACTATA AATACCGCCT CCTTGCGCTC TCTGCCTTCA
4741 TCAATCAAAT CATGTTCTCT CCAATTTTGT CCTTGGAAAT TATTTTAGCT TTGGCTACTT
4801 TGCAATCTGT CTTCGCTGTG CTGTCAAAGT CCTGTGTCAG TCACTTTAGA AATGTTGGAT
4861 CCTTGAATAG TAGGGATGTC AATCTGAAAG ATGACTTTTC CTATGCTAAT ATTGATGATC
4921 CCTATAACAA GCCTTTCGTC CTAAATAACC TAATAAACCC TACCAAGTGT CAAGAGATCA
4981 TGCAATTTGC CAATGGCAAG TTGTTTGACT CCCAAGTCCT GAGTGGCACG GACAAGAACA
5041 TACGTAACTC TCAACAAATG TGGATATCCA AGAACAACCC TATGGTAAAA CCCATTTTCG
5101 AGAACATATG CAGGCAGTTT AACGTACCCT TTGATAATGC CGAGGACCTA CAGGTCGTCC
5161 GTTACTTGCC TAATCAATAT TATAATGAGC ATCATGACTC ATGCTGTGAC TCCTCCAAGC
5221 AATGCAGTGA ATTTATAGAG AGGGGCGGTC AGAGGATTCT GACCGTTTTA ATTTACCTAA
5281 ACAACGAGTT CTCAGATGGA CACACGTACT TTCCTAATTT AAACCAAAAG TTCAAGCCCA
5341 AGACTGGTGA TGCTTTGGTT TTTTACCCTT TAGCCAACAA CTCTAATAAA TGTCACCCAT
5401 ACAGTCTACA CGCAGGTATG CCCGTCACGT CAGGAGAGAA GTGGATTGCT AATCTGTGGT
5461 TTCGTGAGCG TAAGTTCTCC CACCACCACC ACCACCACTA ATGAAGATCT GGAGGAGGCT
5521 GAGGAACCTG ATCTTGAGGA GGATGACGAC CAGAAGGCAG TCAAAGATGA ACTGTGATAA
5581 GGGGGGCCGC GAGTCGTGAG TAATCAAGAG GATGTCAGAA TGCCATTTGC CTGAGAGATG
5641 CAGGCTTCAT TTTTGATACT TTTTTATTTG TAACCTATAT AGTATAGGAT TTTTTTTGTC
5701 ATTTTGTTTC TTCTCGTACG AGCTTGCTCC TGATCAGCCT ATCTCGCAGC TGATGAATAT
5761 CTTGTGGTAG GGGTTTGGGA AAATCATTCG AGTTTGATGT TTTTCTTGGT ATTTCCCACT
5821 CCTCTTCAGA GTACAGAAGA TTAAGTGAGA CGTTCGTTTG TGCTCCGGA

SEQ ID NO 15: primer

GAGCTCGGTACCATGCACCACCACCACCACCACGTGCTGTCAAAGTCCTGTGTCAGTCAC

SEQ ID NO 16: primer

AAGCTTGAATTCTTAGGAGAACTTACGCTCACGAAACCACA

SEQ ID NO 17: primer

GAGCTCGGTACCATGGTGCTGTCAAAGTCCTGTGTCAGTC

SEQ ID NO 18: primer

AAGCTTGAATTCTTAGTGGTGGTGGTGGTGGTGGGAGAACTTACGCTCACGAAACCAC

SEQ ID NO 19: MM-0579

CTCTGCCTTCATCAATCAAATCATGagattcccatctattttcaccgctg

SEQ ID NO 20: MM-0580

AGCTTCGGCCTCTCTTTTCTCGAGA

SEQ ID NO 21: MM-1569

TCTCGAGAAAAGAGAGGCCGAAGCTGTGCTGTCAAAGTCCTGTGTCAGTCACTTT

SEQ ID NO 22: MM-1570

GCAAATGGCATTCTGACATCCTCTTGATTAGTGGTGGTGGTGGTGGTGGGAGAACTT
ACG

SEQ ID NO 23: MM-0784

AGGAGGCCATGCACATTGTCAGAATTAGAAGGTTCTGGCTCTGGTTCTGGCTCT
ATGAGATTCCCATCTATTTTCACCGCTGTC

SEQ ID NO 24: Protein sequence in PP681

MFSPILSLEIILALATLQSVFAQQEAVDGGCSHLGQSYADRDVWKPEPCQICVCDSGSVL
CDDIICDDQELDCPNPEIPFGECCAVCPQPPTAPTRPPNGQGPQGPKGDPGPPGIPGRNGD
PGPPGSPGSPGSPGPPGICESCPTGGQNYSPQYEAYDVKSGVAGGGIAGYPGPAGPPGPP
GPPGTSGHPGAPGAPGYQGPPGEPGQAGPAGPPGPPGAIGPSGPAGKDGESGRPGRPGER
GFPGPPGMKGPAGMPGFPGMKGHRGFDGRNGEKGETGAPGLKGENGVPGENGAPGPM
GPRGAPGERGRPGLPGAAGARGNDGARGSDGQPGPPGPPGTAGFPGSPGAKGEVGPAG
SPGSSGAPGQRGEPGPQGHAGAPGPPGPPGSNGSPGGKGEMGPAGIPGAPGLIGARGPPG
PPGTNGVPGQRGAAGEPGKNGAKGDPGPRGERGEAGSPGIAGPKGEDGKDGSPGEPGA
NGLPGAAGERGVPGFRGPAGANGLPGEKGPPGDRGGPGPAGPRGVAGEPGRDGLPGGP
GLRGIPGSPGGPGSDGKPGPPGSQGETGRPGPPGSPGPRGQPGVMGFPGPKGNDGAPGK
NGERGGPGGPGPQGPAGKNGETGPQGPPGPTGPSGDKGDTGPPGPQGLQGLPGTSGPPG
ENGKPGEPGPKGEAGAPGIPGGKGDSGAPGERGPPGAGGPPGPRGGAGPPGPEGGKGAA
GPPGPPGSAGTPGLQGMPGERGGPGGPGPKGDKGEPGSSGVDGAPGKDGPRGPTGPIGP
PGPAGQPGDKGESGAPGVPGIAGPRGGPGERGEQGPPGPAGFPGAPGQNGEPGAKGERG
APGEKGEGGPPGAAGPAGGSGPAGPPGPQGVKGERGSPGGPGAAGFPGGRGPPGPPGSN
GNPGPPGSSGAPGKDGPPGPPGSNGAPGSPGISGPKGDSGPPGERGAPGPQGPPGAPGPL
GIAGLTGARGLAGPPGMPGARGSPGPQGIKGENGKPGPSGQNGERGPPGPQGLPGLAGT
AGEPGRDGNPGSDGLPGRDGAPGAKGDRGENGSPGAPGAPGHPGPPGPVGPAGKSGDR
GETGPAGPSGAPGPAGSRGPPGPQGPRGDKGETGERGAMGIKGHRGFPGNPGAPGSPGP
AGHQGAVGSPGPAGPRGPVGPSGPPGKDGASGHPGPIGPPGPRGNRGERGSEGSPGHPG
QPGPPGPPGAPGPCCGAGGVAAIAGVGAEKAGGFAPYYG

We claim:

1. A yeast host cell comprising a recombinant monomeric prolyl 4-hydroxylase.

2. The yeast host cell of claim 1, wherein the monomeric prolyl 4-hydroxylase is secreted.

3. The yeast host cell of claim 1 or claim 2, wherein the recombinant monomeric prolyl 4-hydroxylase is from a virus, algae, or a plant.

4. The yeast host cell of any one of claims 1-3, wherein the recombinant monomeric prolyl 4-hydroxylase is from mimivirus.

5. The yeast host cell of any one of claims 1-3, wherein the recombinant monomeric prolyl 4-hydroxylase is from Arabidopsis thaliana.

6. The yeast host cell of any one of claims 1-3, wherein the recombinant monomeric prolyl 4-hydroxylase is from C. reinhardtii.

7. The yeast host cell of any one of claims 1-3, wherein the recombinant monomeric prolyl 4-hydroxylase is from Paramecium bursaria Chlorella virus-1.

8. The yeast host cell of any one of claims 1-7, wherein the recombinant monomeric prolyl 4-hydroxylase is at least 80% identical to a prolyl 4-hydroxylase selected from the group consisting of: SEQ ID NOs: 2, 3, 6, 7 and 8.

9. The yeast host cell of any one of claims 1-8, wherein the yeast is Pichia.

10. The yeast host cell of any one of claims 1-9, further comprising a second protein to be hydroxylated.

11. The yeast host cell of claim 10, wherein the second protein is selected from the group consisting of: collagen, recombinant collagen, and collagen-like proteins.

12. A microorganism comprising a recombinant monomeric prolyl 4-hydroxylase, wherein the recombinant monomeric prolyl 4-hydroxylase is from algae or a plant.

13. The microorganism of claim 12, wherein the monomeric prolyl 4-hydroxylase is secreted.

14. The microorganism of claim claim 12 or claim 13, wherein the recombinant monomeric prolyl 4-hydroxylase is from Arabidopsis thaliana.

15. The microorganism of claim 12 or claim 13, wherein the recombinant monomeric prolyl 4-hydroxylase is from C. reinhardtii.

16. The microorganism of any one of claims 12-15, wherein the recombinant monomeric prolyl 4-hydroxylase is at least 80% identical to a prolyl 4-hydroxylase selected from the group consisting of: SEQ ID NOs: 7 and 8.

17. The microorganism of any one of claims 12-16, wherein the microorganism is a yeast or a bacteria.

18. The microorganism of claim 17, wherein the microorganism is E. coli.

19. The microorganism of claim 17, wherein the microorganism is Pichia.

20. The microorganism of any one of claims 12-19, further comprising a second protein to be hydroxylated.

21. The microorganism of claim 20, wherein the second protein is selected from the group consisting of: collagen, recombinant collagen, and collagen-like proteins.

22. A method of producing a recombinant monomeric prolyl 4-hydroxylase, comprising purifying the recombinant monomeric prolyl 4-hydroxylase from the yeast host cell of any one of claims 1-11.

23. A method of producing a recombinant monomeric prolyl 4-hydroxylase, comprising purifying the recombinant monomeric prolyl 4-hydroxylase from the microorganism of any one of claims 12-21.

24. An in vitro method for hydroxylating a protein comprising: lysing a microorganism comprising a protein to be hydroxylated to create a lysate; adding a specific concentration of a monomeric prolyl 4-hydroxylase purified from the yeast host cell of any one of claims 1-11; and incubating the lysate and the monomeric prolyl 4-hydroxylase in reaction conditions that promote the hydroxylation of the protein by the monomeric prolyl 4-hydroxylase.

25. An in vitro method for hydroxylating a protein comprising: lysing a first microorganism comprising a protein to be hydroxylated to create a lysate; adding a specific concentration of a monomeric prolyl 4-hydroxylase purified from the microorganism of any one of claims 12-21 to the lysate; and incubating the lysate and the monomeric prolyl 4-hydroxylase in reaction conditions that promote the hydroxylation of the protein by the monomeric prolyl 4-hydroxylase.

26. An in vitro method for hydroxylating a protein comprising: adding a specific concentration of a monomeric prolyl 4-hydroxylase purified from the yeast host cell of any one of claims 1-11 to a reaction mixture; adding a specific concentration of a protein to be hydroxylated to the reaction mixture; and incubating the reaction micture in reaction conditions that promote the hydroxylation of the protein by the monomeric prolyl 4-hydroxylase.

27. An in vitro method for hydroxylating a protein comprising: adding a specific concentration of a monomeric prolyl 4-hydroxylase purified from the microorganism of any one of claims 12-21 to a reaction mixture; adding a specific concentration of a protein to be hydroxylated to the reaction mixture; and incubating the reaction micture in reaction conditions that promote the hydroxylation of the protein by the a monomeric prolyl 4-hydroxylase.

28. An ex vivo method for hydroxylating a protein comprising: lysing the microorganism of any one of claims 12-21 to create a lysate; incubating the lysate and a recombinant protein to be hydroxylated in reaction conditions that promote the hydroxylation of the protein by the monomeric prolyl 4-hydroxylase.

29. An ex vivo method for hydroxylating a protein comprising: lysing the yeast host cell of any one of claims 1-11 to create a lysate; incubating the lysate and a recombinant protein to be hydroxylated e in reaction conditions that promote the hydroxylation of the protein by the monomeric prolyl 4-hydroxylase.

30. An ex vivo method for hydroxylating a protein comprising: lysing the microorganism of any one of claims 12-21, comprising a recombinant monomeric prolyl 4-hydroxylase to create a lysate; lysing a second microorganism comprising a protein to be hydroxylated to create a lysate; and incubating the lysate of the first microorganism and the lysate of the second microorganism in reaction conditions that promote the hydroxylation of the protein by the monomeric prolyl 4-hydroxylase.

31. An ex vivo method for hydroxylating a protein comprising: lysing the yeast host cell of any one of claims 1-11, comprising a recombinant monomeric prolyl 4-hydroxylase; to create a lysate; lysing a microorganism comprising a protein to be hydroxylated to create a lysate; and incubating the lysate of yeast host cell and the lysate of the microorganism in reaction conditions that promote the hydroxylation of the protein by the monomeric prolyl 4-hydroxylase.
